(19) United States
(12) Patent Application Publication
(10) Pub. No.: US 2024/0180881 A1
(43) Pub. Date: Jun. 6, 2024

(54) METHODS FOR DECREASING RESISTANCE TO CHEMOTHERAPY

(71) Applicant: National Institute of Immunology, Delhi (IN)

(72) Inventor: Sagar SENGUPTA, Delhi (IN)

(21) Appl. No.: 18/513,311

(22) Filed: Nov. 17, 2023

Related U.S. Application Data
(60) Provisional application No. 63/426,360, filed on Nov. 17, 2022.

(51) Int. Cl. A61K 31/433; A61K 31/282; A61K 31/4745; A61K 31/513; A61K 31/519; A61K 31/553; A61K 31/7068; A61K 31/7072; A61K 33/243; A61P 35/00

(52) U.S. Cl. CPC A61K 31/433; A61K 31/282; A61K 31/4745; A61K 31/513; A61K 31/519; A61K 31/553; A61K 31/7068; A61K 31/7072; A61K 33/243; A61P 35/00

(57) ABSTRACT

The present disclosure shows that a stretch of 32 amino acids in BLM interacts with RAD54 and this interaction contributes to resistance against chemotherapeutic drugs such as cisplatin, camptothecin, oxaliplatin, etc. in cancer cells. The present disclosure provides an inhibitor of BML-RAD54 interaction as an adjunct therapy for treating cancer in a patient, wherein the patient is receiving primary chemotherapy. In some embodiments, the inhibitor of BML-RAD54 interaction is selected from Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, or a combination thereof.

### CROSS-REFERENCE TO RELATED APPLICATIONS

The present application claims the benefit of priority to U.S. Provisional Application No. 63/426,360, filed on Nov. 17, 2022, the contents of which are hereby incorporated by reference herein in its entirety.

### FIELD OF THE DISCLOSURE

The present disclosure relates to methods for treating cancer such as colon cancer. In particular, the present disclosure relates to methods for decreasing chemoresistance in colon cancer cells by disrupting BLM-RAD54 interaction within the cancer cells.

### BACKGROUND OF THE DISCLOSURE

Dysregulation of one or more DNA repair pathways has been associated with tumor initiation and progression. As the rate of proliferation of cancer cells is much higher than that of normal cells, cancer therapy usually involves utilization of DNA damaging agents that more efficiently eliminate cancer cells than normal tissue cells. However, the efficacy of these toxic agents can be modulated by cancer cells by sensing the damaged DNA and by repairing the damaged DNA (Li et al., “DNA Repair Pathways in Cancer Therapy and Resistance”. Front Pharmacol. 2020; 11:629266). This heightened DNA repair capacity in cancer cells has been implicated in therapy resistance and thus poses a major challenge in the management of cancer (Sakthivel and Hariharan, “Regulatory players of DNA damage repair mechanisms: Role in Cancer Chemoresistance.” Biomed Pharmacother. 2017; 93:1238-45).

Thus, the treatment of cancer is associated with a major drawback that tumors/cancer cells show a diminished response (i.e., develop resistance) over a period of time to chemotherapeutic agents and/or radiation therapy. Indeed, this is one of the main reasons why, despite progress in chemotherapy, many cancers are still resistant to effective chemotherapeutic intervention. For example, resistance to chemotherapeutic agents/drugs is one of the main causes of poor treatment outcome in colon cancer patients. Chemoresistance can occur due to hyper-active DNA repair system which does not allow DNA breaks made by chemotherapeutic drugs (like cisplatin, camptothecin, oxaliplatin or their next generation derivatives) to persist in the cells and thereby rescues cancer cells from apoptosis. In particular, Homologous Recombination (HR) repair has been implicated in cancer development and drug resistance (Helleday, “Homologous recombination in cancer development, treatment and development of drug resistance.” Carcinogenesis. 2010; 31(6):955-60).

Bloom syndrome protein (BLM) is a multi-functional protein that functions both during DNA damage sensing and DNA repair (Kaur et al., “Functions of BLM Helicase in Cells: Is It Acting Like a Double-Edged Sword?” Front Genet. 2021; 12:634789). During DNA damage sensing, BLM functionally interacts with multiple key proteins during DNA damage response (Tripathi et al. “MRN complex-dependent recruitment of ubiquitylated BLM helicase to DSBs negatively regulates DNA repair pathways.” Nature communications. 2018; 9(1):1016). During the repair phase, BLM functions in the repair of DNA double strand breaks (DSBs) particularly during HR using several different mechanisms (Kaur et al. “Functions of BLM Helicase in Cells: Is It Acting Like a Double-Edged Sword?” Front Genet. 2021; 12:634789). A lack of functional BLM protein has been associated with a rare genetic disorder Bloom Syndrome (BS) (Ellis et al. “The Bloom's syndrome gene product is homologous to RecQ helicases.” Cell. 1995; 83(4):655-66). Typical characteristics of BS patients include increased sensitivity towards DNA-damaging agents including hydroxyurea (HU), camptothecin and ionizing radiation, thereby predisposing these patients to a wide spectrum of cancers including solid cancers, leukemias and lymphomas (Cunniff et al. “Bloom's Syndrome: Clinical Spectrum, Molecular Pathogenesis, and Cancer Predisposition.” Mol Syndromol. 2017; 8(1):4-23). Recent reports have suggested that in multiple cancers including colon cancer, BLM protein is aberrantly overexpressed and the aberrant expression has been linked to poor patient outcome (Kaur et al., “Functions of BLM Helicase in Cells: Is It Acting Like a Double-Edged Sword?” Front Genet. 2021; 12:634789). Another core factor in the HR pathway, RAD54, is involved in multiple crucial steps in this process in concert with the central homologous pairing protein, RAD51 (Heyer et al. “Rad54: the Swiss Army knife of homologous recombination?” Nucleic Acids Res. 2006; 34(15):4115-25).

Remodeling of chromatin occurs at different steps during DNA damage response. RAD54 has been demonstrated to function as a chromatin remodeler, both in vitro (Alexeev et al. “Rad54 protein possesses chromatin-remodeling activity stimulated by the Rad51-ssDNA nucleoprotein filament.” Nat Struct Biol. 2003; 10(3):182-6) and in cellulo (Wolner and Peterson. “ATP-dependent and ATP-independent roles for the Rad54 chromatin remodeling enzyme during recombinational repair of a DNA double strand break.” J Biol Chem. 2005; 280(11):10855-60). The remodeling complexes also play a critical role in repositioning of the nucleosomes immediately after exposure to DNA damage in order to provide repair enzymes access to the damaged DNA and thereby ensuring that the response pathway becomes operative and thus prevent genomic alterations (Lans et al., “ATP-dependent chromatin remodeling in the DNA-damage response. Epigenetics Chromatin.” 2012; 5:4; Stadler and Richly. “Regulation of DNA Repair Mechanisms: How the Chromatin Environment Regulates the DNA Damage Response.” Int J Mol Sci. 2017; 18(8)). It has been shown that the N-terminal (1-212) region of BLM enhanced the chromatin remodeling activity of RAD54 (Srivastava et al. “BLM helicase stimulates the ATPase and chromatin-remodeling activities of RAD54.” J Cell Sci. 2009; 122(Pt 17):3093-103).

Prior studies indicate that RAD54 and BLM helicase play pivotal roles during homologous recombination repair ensuring genome maintenance. The present disclosure explores whether BLM-RAD54 interaction plays a role in development of resistance to chemotherapy in cancer cells and provides treatment methods comprising administering one or more small molecule which can disrupt or inhibit BLM-RAD54 interaction or complex formation as an adjunct therapy to treat cancer by reducing resistance to chemotherapy in cancer cells and the like.

### SUMMARY OF THE DISCLOSURE

The present disclosure provides a method for treating cancer in a patient in need thereof, comprising: a) administering a chemotherapy to the patient; and b) administering an inhibitor of Bloom syndrome protein (BLM) and RAD54 interaction to the patient after starting or simultaneously with the chemotherapy. In some embodiments, the cancer treated by the methods of the present disclosure is a cancer where cancer cells develop resistance to primary treatment such as chemotherapy due to BLM-RAD54 interaction in cancer cells.

The present disclosure further provides a method for treating colorectal cancer in a patient in need thereof, comprising: a) administering a chemotherapy to the patient; and b) administering an inhibitor of BLM and RAD54 interaction to the patient after starting or simultaneously with the chemotherapy.

The present disclosure further provides a method for inhibiting an interaction of BLM and RAD54 in cancer cells, comprising contacting the cancer cells with an inhibitor of BLM-RAD54 interaction selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.

The present disclosure provides a method for reducing resistance to chemotherapy in a cancer patient, comprising administering an inhibitor of BLM-RAD54 interaction to the cancer patient after starting or along with the chemotherapy.

The present disclosure provides an inhibitor of BLM and RAD54 interaction for use as an adjunct therapy in a method for treating cancer. In some embodiments, the inhibitor of BLM-RAD54 interaction is selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof. In some embodiments, an inhibitor of BLM-RAD54 interaction is selected from the group consisting of: Acetazolamide or a pharmaceutically acceptable salt thereof, Dipyridamole or a pharmaceutically acceptable salt thereof, Loxapine Succinate, or a combination thereof. In some embodiments, the method for treating cancer comprises administration of one or more chemotherapeutic agents (such as camptothecin, oxaliplatin and cisplatin or their next generation derivative). In some embodiments, the method for treating cancer comprises administration of cisplatin, oxaloplatin, carboplatin, camptothecin, 5-Fluorouracil (5-FU), Capecitabine, Irinotecan, Trifluridine, tipiracil, or a combination thereof.

### BRIEF DESCRIPTION OF THE DRAWINGS

FIG. 1A shows levels of BLM and RAD54 proteins in colon cancer cells (HCT116) and normal colon epithelial cells (CCD 841 CoN).

FIG. 1B shows the results of proximity ligation assay to determine the interaction between BLM and RAD54 in colon cancer cells (HCT116) and normal colon epithelial cells (CCD 841 CoN).

FIG. 1C shows the quantitation of the data in FIG. 1B.

FIG. 2A shows the results of a Renilla luciferase-based protein complementation assay (PCA) demonstrating that the N-terminal region of RAD54 interacts in cellulo with BLM (181-212).

FIG. 2B shows the results of an immunoprecipitation experiment demonstrating that BLM (181-212) interacts with endogenous RAD54 in cellulo.

FIG. 2C shows a schematic diagram of G5E4 array (upper panel) and pictures of Coomassie gels demonstrating the purified GST BLM (1-212), GST RAD54 (1-747) (WT), GST BLM (1-1417) (WT), His RAD54 (1-747) (WT) (lower panel).

FIG. 2D shows the results of restriction enzyme accessibility (REA) assays carried out with chromatinized G5E4 array using recombinant RAD54 and RAD54+BLM (1-212) (upper panel) and RAD54+BLM peptide (181-212) (BLM_peptide) or RAD54+a scrambled peptide (termed SCM_peptide) (lower panel). All reactions were done in presence of ATP.

FIG. 2E shows the quantification of the data in FIG. 2D.

FIG. 2F shows the quantitation of the ATP binding assays that were carried out using RAD54 WT, RAD54 WT+BLM_peptide, RAD54 WT+SCM_peptide demonstrating that the BLM (181-212) enhances the ATP binding capacity of RAD54.

FIG. 2G shows the quantitation of the ATPase activity carried out using RAD54 WT, BLM (1-212), BLM_peptide, SCM_peptide, RAD54 WT+a gradient of BLM (1-212) (180 nM, 360 nM, 540 nM), RAD54 WT+a gradient of BLM_peptide (180 nM, 360 nM, 540 nM), RAD54 WT+a gradient of SCM_peptide (180 nM, 360 nM, 540 nM) demonstrating that the BLM (181-212) peptide increased the ATPase activity of RAD54.

FIG. 2H shows the results of the tryptophan fluorescence assays that were carried out with RAD54 WT or RAD54 WT in the presence of the indicated concentrations of either BLM_peptide (upper panel) or SCM_peptide (lower panel) demonstrating that the BLM (181-212) peptide altered the conformation of RAD54.

FIG. 2I shows the results of an in vitro interaction assay between the bound GST or GST-BLM wildtype or GST BLM (Delta 181-212) and soluble His RAD54.

FIG. 2J: The left panel shows the results of immunoblotting in an in vivo interaction assay performed with indicated antibodies. The right panel shows the results of immunoprecipitation in an in vivo interaction assay performed with indicated antibodies.

FIG. 2K shows the results of a chromatin remodeling assay carried out under the following conditions: RAD54 wildtype alone (−ATP), RAD54 wildtype alone (+ATP), RAD54 wildtype+BLM wildtype (+ATP), RAD54 wildtype+BLM (Delta 181-212) (+ATP)

FIG. 2L shows the quantitation of the data in FIG. 2K.

FIG. 3A shows the results of the cellular uptake of TAMRA tagged BLM_CPP and SCM_CPP into GM03509 GFP cells.

FIG. 3B shows the results of Western blotting showing levels of RAD51, RAD54 and γH2AX in lysates of GM03509 GFP cells grown in presence of HU (for 16 hours) or for 6 hours more after washing away HU in presence of 180 nM BLM_CPP or SCM_CPP.

FIG. 3C shows the results of Western blotting showing levels of RAD51, RAD54 and γH2AX in lysates of GM03509 GFP cells grown in presence of HU (for 16 hours) or for 6 hours more after washing away HU in presence of BLM_NP or SCM_NP.

FIG. 3D shows the results of a quantitation of RAD51 and RAD54 foci numbers in an immunofluorescence experiment where HCT116 BLM−/− cells were grown in presence of HU (for 16 hours) and for 6 hours more after washing away HU in presence of 180 nM BLM_CPP or SCM_CPP.

FIG. 3E shows the results of a flow cytometry experiment demonstrating that the BLM CPP allowed the cells to enter into proliferation mode much earlier than SCM_CPP.

FIG. 3F shows the results of a Western blot analysis demonstrating that the levels of cyclin-dependent kinase inhibitors, p21 and p27 were reduced when cells were treated with BLM_CPP.

FIG. 3G shows the results of a Western analysis demonstrating that the levels of cyclin-dependent kinase inhibitors, p21 and p27 were reduced when cells were treated with BLM_NP.

FIG. 3H shows the results of an immunofluorescence experiment where HCT116 BLM−/− cells were grown in presence of HU (for 16 hours) and for 6 hours more after washing away HU in presence of 180 nM BLM_CPP or SCM_CPP. The left panel shows representative images and the right panel shows the quantitation of γH2AX foci numbers.

FIG. 3I shows the results of Comet assay demonstrating that the BLM_CPP decreased the levels of cellular DNA damage.

FIG. 3J shows the results of MTT assays demonstrating that the cellular resistance to cisplatin was increased due to BLM_CPP.

FIG. 3K shows the results of MTT assays demonstrating that the cellular resistance to camptothecin was increased due to BLM_CPP.

FIG. 4A depicts that BLM and RAD54 were highly enriched only on MRP2 promoter in resistant cells as determined by ChIP assay

FIG. 4B shows the results of Re-ChIP experiments demonstrating that the BLM and RAD54 were both co-recruited onto the MRP2 promoter with higher occupancy in the resistant cells as compared to the wild type cells.

FIG. 4C shows increased ATP-dependent chromatin remodelling of the chromatinized MRP2 array in the presence of BLM (1-212).

FIG. 4D shows the quantitation of enhanced ATP-dependent RAD54 mediated chromatin remodelling.

FIG. 4E shows the results of an experiment showing increased transcription of multiple MDR genes (including MRP2) in HCT116 IC60 CPTR cells demonstrating enhanced remodeling by BLM/RAD54 complex.

FIG. 4F shows the results of MRP2 activity demonstrating that the HCT116 WT IC60 CPTR cells have enhanced MRP2 efflux activity.

FIG. 4G shows that the number of colonies formed during invasion assays was increased in presence of BLM-CPP after camptothecin (CPT) treatment.

FIG. 4H shows enhanced tumor growth in a xenograft mouse model after the treatment with CPT-BLM-Gel.

FIG. 4I shows that the BLM (181-212) region enhanced tumor growth in xenograft mouse model.

FIG. 5A shows the results of a Renilla luciferase-based protein complementation assay (PCA) demonstrating the decrease in the Renilla luciferase activity due to disruption of the BLM-RAD54 interaction by small molecules present in the Prestwick library.

FIG. 5B shows an in vitro interaction assays results demonstrating that three compounds (C3, C7, C17) disrupted the RAD54-BLM interaction.

FIG. 5C shows the results of REA assays indicating that C3, C7, C17 decreased the efficiency of BLM dependent enhancement of RAD54 chromatin remodeling activity.

FIG. 5D shows the quantitation of the experiment in FIG. 5C.

FIG. 5E shows the quantitation of the ATP binding assays demonstrating that C3, C7, C17 decreased the BLM dependent enhancement of the binding of ATP by RAD54.

FIG. 5F shows the quantitation of the ATPase activity demonstrating that C3, C7, C17 decreased the BLM dependent enhancement of the ATPase activity of RAD54.

FIG. 5G shows the results of Tryptophan fluorescence assays demonstrating that the C17 altered the conformation of RAD54.

FIG. 5H shows the results of Octet BLI based studies to determine the dissociation constant (KD) of the interaction of different concentrations of Biotin BLM_Peptide (left panel) and C17 (right panel) with His-RAD54 WT immobilized onto Ni-NTA-sensor. The KD of binding is indicated.

FIG. 6A shows decreased levels of homologous recombination (HR) in HCT116 IC60 CPTR cells due to C3, C7, C17.

FIG. 6B shows the results of soft agar assays demonstrating that C3, C7, C17 decreased anchorage independent cell growth of HCT116 IC60 CPTR cells.

FIG. 6C shows that C3 decreased tumor formation by camptothecin (left panel) and oxaliplatin (right panel) resistant cells in xenograft model in SCID mice (left panel) and in NSG mice (right panel).

FIG. 6D shows that C7 decreased tumor formation by camptothecin (left panel) and oxaliplatin (right panel) resistant cells in xenograft model using either SCID mice (left panel) and in NSG mice (right panel).

FIG. 6E shows that C17 decreased tumor formation by camptothecin (left panel) and oxaliplatin (right panel) resistant cells in xenograft model using either SCID mice (left panel) and in NSG mice (right panel).

FIG. 6F shows the results of RT-qPCR from tumors obtained at the end point of the xenograft experiment indicating decreased MRP2 transcript levels upon treatment with CPT and C17.

FIG. 6G shows the results of Western blotting of lysates obtained from tumors at the end point of the xenograft experiment with an anti-MRP2 antibody indicating decreased MRP2 protein levels upon treatment with CPT and C17.

FIG. 7A shows the volume of tumors generated in SCID mice using HCT116 IC60 CPTR cells and injected with siControl at the base of the tumor once the tumor volume reached 50 mm³ and left untreated or treated with CPT alone, C17 alone, or a combination of C17 and CPT.

FIG. 7B shows the volume of tumors generated in SCID mice using HCT116 IC60 CPTR cells and injected with siMRP2 at the base of the tumor once the tumor volume reached 50 mm³ and left untreated or treated with CPT alone, C17 alone, or a combination of C17 and CPT.

FIG. 7C shows the volume of tumors generated in SCID mice using HCT116 IC60 CPTR cells and injected with siRAD54 at the base of the tumor once the tumor volume reached 50 mm³ and left untreated or treated with CPT alone, C17 alone, or a combination of C17 and CPT.

FIG. 7D shows the volume of tumors generated in SCID mice using HCT116 IC60 CPTR cells and injected with siBLM at the base of the tumor once the tumor volume reached 50 mm³ and left untreated or treated with CPT alone, C17 alone, or a combination of C17 and CPT.

FIG. 7E indicates the mechanism RAD54-BLM mediated chemoresistance and its disruption by C3, C7, C17.

FIG. 8A shows the results of Western analysis of BLM in lysates made from HCT116 WT and HCT116 BLM−/−.

FIG. 8B shows the results of MTT assays in HCT116 WT and HCT116 BLM−/− cells treated with either BLM_CPP or SCM_CPP after CDDP treatment.

FIG. 8C shows the results of MTT assays in HCT116 WT and HCT116 BLM−/− cells treated with either BLM_CPP or SCM_CPP after CPT treatment.

FIG. 8D shows the results of MTT assays in HCT116 WT and HCT116 BLM−/− cells treated with either BLM_NP or SCM_NP after CPT treatment.

FIG. 8E shows the results of Western analysis of BLM in lysates made from DLD1 cells treated with either siControl or siBLM.

FIG. 8F shows the results of MTT assays in DLD1 siControl and DLD1 siBLM cells treated with either BLM_CPP or SCM_CPP after CPT treatment.

FIG. 8G shows the results of Western analysis of BLM in lysates made from HT-29 cells treated with either siControl or siBLM.

FIG. 8H shows the results of MTT assays in HT-29 siControl and HT-29 siBLM cells treated with either BLM_CPP or SCM_CPP after CPT treatment.

FIG. 8I shows the results of Western analysis of BLM in lysates made from SW480 cells treated with either siControl or siBLM.

FIG. 8J shows the results of MTT assays in SW480 siControl and SW480 siBLM cells treated with either BLM_CPP or SCM_CPP after CPT treatment.

FIG. 8K shows the results of Western analysis of BLM in lysate from SW620 cells treated with either siControl or siBLM.

FIG. 8L shows the results of MTT assays in SW620 siControl and SW620 siBLM cells treated with either BLM_CPP or SCM_CPP after CPT treatment.

FIG. 9A shows the results of Western blot analysis demonstrating that the GM03509 BLM Clone 9.6 cells express BLM.

FIG. 9B shows the results of immunofluorescence with anti-BLM antibody demonstrating GM03509 BLM Clone 9.6 cells form BLM foci post HU treatment.

FIG. 9C depicts representative images for Sister Chromatin Exchanges (SCEs) performed in GM03509 and GM03509 BLM Clone 9.6 cells.

FIG. 9D shows the quantitation of the data in FIG. 9C.

FIG. 10A represents IGV browser tracks from BLM peaks and input signals within 566 bp from TSS of MDR gene promoters with recruited BLM: MRP2, MRP3, MRP4, MRP5, MXR, BSEP, ABCA2 and ABCG5 (upper panel) and MDR gene promoters not recruited BLM: MRP1, MDR1 (lower panel).

FIG. 10B shows the results of REA assays showing that RAD54 enhanced chromatin remodeling equally on G5E4 and MRP2 arrays.

FIG. 10C shows the quantitation of the data in FIG. 10B.

FIG. 10D shows the results of REA assays demonstrating that the BLM_peptide enhances the RAD54 mediated chromatin remodelling on MRP2 arrays.

FIG. 10E shows the quantitation of the data in FIG. 10D.

FIG. 11A shows the dose response curve for C3, C7, and C17 when tested for BLM-RAD54 complex disruption.

FIG. 11B shows the results of Western blotting carried out with antibodies against BLM, RAD54, beta Actin on lysates made from HEK293T cells either left untreated or treated with 100 nM C3, C7, C17 for 72 hours (left panel) and the results of immunoprecipitation (IP) carried out with anti-BLM antibody (right panel).

FIG. 11C shows the results of REA assays that were carried out with chromatinized G5E4 array demonstrating that C3, C7, C17 decrease the efficiency of BLM dependent enhancement of RAD54 chromatin remodelling activity.

FIG. 11D shows the quantitation of the data in FIG. 11C.

FIG. 11E shows the results of tryptophan fluorescence assays demonstrating that C3 (Top) and C7 (Bottom) alter the conformation of RAD54.

FIG. 11F shows the results of Octet BLI based studies demonstrating that C3 (Top) and C7 (Bottom) bind to RAD54. The KD of binding is indicated.

FIG. 12A shows the results of MTT assays carried out on HCT116 IC60 CPTR or HCT116 cells treated with 100 nM of C3, C7, C17 and increasing concentrations of CPT.

FIG. 12B shows the results of MTT assays carried out on HCT116 IC60 1-OHPR or HCT116 cells treated with 100 nM of C3, C7, C17 and increasing concentrations of 1-OHP.

FIG. 12C shows the results of MTT assays carried out on HCT116 IC60 CDDPR or HCT116 cells treated with 100 nM of C3, C7, C17 and increasing concentrations of CDDP.

FIG. 12D shows the results of RT-qPCR demonstrating that C3, C7, C17 alone do not alter the transcript levels of MDR genes in HCT116 IC60 CPTR cells.

FIG. 12E shows that C3/C7/C17 along with CPT decrease the MRP2 activity in HCT116 WT IC60 CPTR cells.

FIG. 13A shows that the treatment with CPT and C17 decrease the transcript levels of multiple MDR genes in tumor cells obtained from mouse xenograft tissues.

FIG. 13B shows decreasing levels of MDR proteins when tumors cells obtained from mouse xenograft tissues where the tumours were treated with CPT and C17 and probed with antibodies against MRP2, MRP5 and β-actin.

FIG. 13C shows the staining intensity of tumor cells stained with stained with anti-Ki67 antibody in tissues obtained from mouse xenografts where the tumours were treated with C3, C7, C17 (±CPT).

FIG. 13D shows the results of Western blot analysis of PCNA levels in tumor cells obtained from mouse xenografts tissues where the tumours were treated with C3, C7, C17 (±CPT).

FIG. 13E shows the results of a TUNEL assay in tissues obtained from mouse xenografts where the tumours were treated with C3, C7, C17 (±CPT).

FIG. 14A shows the results of RT-qPCR for the levels of MRP2 in tissues obtained from mouse xenografts treated with siControl or siMRP2 and where the tumours were treated with C17 (±CPT).

FIG. 14B shows the results of RT-qPCR for the levels of RDA54 in tissues obtained from mouse xenografts treated with siControl or siRAD54 and where the tumours were treated with C17 (±CPT).

FIG. 14C shows the results of RT-qPCR for the levels of BLM in tissues obtained from mouse xenografts treated with siControl or siBLM and where the tumours were treated with C17 (±CPT).

FIG. 15A shows tumor volumes in xenografts assays performed in NSG mice using HCT116 CPT(R) cells stably expressing BLM (Delta 181-212) in BLM−/− background treated with camptothecin (CPT) or C3 or CPT+C3.

FIG. 15B shows tumor volumes in xenografts assays performed in NSG mice using HCT116 CPT(R) cells stably expressing BLM (Delta 181-212) in BLM−/− background treated with camptothecin (CPT) or C7 or CPT+C7.

FIG. 15C shows tumor volumes in xenografts assays performed in NSG mice using HCT116 CPT(R) cells stably expressing BLM (Delta 181-212) in BLM−/− background treated with camptothecin (CPT) or C17 or CPT+C17.

FIG. 15D shows the RNA levels of the MDR genes in xenografts obtained from tumours from HCT116 CPT(R) cells expressing BLM (Delta 181-212) in BLM−/− background without or with C17 treatment.

FIG. 15E shows the protein levels of the MDR genes in xenografts obtained from tumours from HCT116 CPT(R) cells expressing BLM (Delta 181-212) in BLM−/− background without or with C17 treatment.

FIG. 16A shows tumor volumes in xenografts assays performed in NSG mice using HT-29 OHP(R) cells treated with camptothecin (CPT) or C3 or CPT+C3.

FIG. 16B shows tumor volumes in xenografts assays performed in NSG mice using HT-29 OHP(R) cells treated with camptothecin (CPT) or C7 or CPT+C7.

FIG. 16C shows tumor volumes in xenografts assays performed in NSG mice using HT-29 OHP(R) cells treated with camptothecin (CPT) or C17 or CPT+C17.

FIG. 16D shows the RNA levels of the MDR genes in xenografts obtained in tumours from HT-29 OHP(R) cells without or with C17 treatment.

FIG. 16E shows the protein levels of the MDR genes in xenografts obtained in tumours from HT-29 OHP(R) cells without or with C17 treatment.

FIG. 17A shows tumor volumes in xenografts assays performed in BALB/c mice using CT26 cells treated with camptothecin (CPT) or C3 or CPT+C3.

FIG. 17B shows tumor volumes in xenografts assays performed in BALB/c mice using CT26 cells treated with camptothecin (CPT) or C7 or CPT+C7.

FIG. 17C shows tumor volumes in xenografts assays performed in BALB/c mice using CT26 cells treated with camptothecin (CPT) or C17 or CPT+C17.

FIG. 17D shows the RNA levels of the MDR genes obtained from a syngeneic or allogenic model using CT26 cells without or with C17 treatment.

FIG. 17E shows the protein levels of the MDR genes obtained from a syngeneic or allogenic model using CT26 cells without or with C17 treatment.

### DETAILED DESCRIPTION OF THE DISCLOSURE

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression “at least” or “at least one” suggests the use of one or more elements or ingredients or quantities, as the use may be in the embodiment of the disclosure to achieve one or more of the desired objects or results. Throughout this specification, the word “comprise”, or variations such as “comprises” or “comprising” or “containing” or “has” or “having”, or “including but not limited to” wherever used, will be understood to imply the inclusion of a stated element, integer or step, or group of elements, integers or steps, but not the exclusion of any other element, integer or step, or group of elements, integers or steps.

Reference throughout this specification to “one embodiment”, “an embodiment”, or “some embodiments” means that a particular feature, structure or characteristic described in connection with the embodiment may be included in at least one embodiment of the present disclosure. Thus, the appearances of the phrases “in one embodiment”, “in an embodiment”, or “in some embodiments” in various places throughout this specification may not necessarily all refer to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination.

The term “subject” or “patient” as used herein refers to any mammal including, without limitation, humans and other primates (e.g., chimpanzees and other apes and monkey species), farm animals (e.g., cattle, sheep, pigs, goats and horses), domestic mammals (e.g., dogs and cats), and laboratory animals (e.g., rodents such as mice, rats, and guinea pigs). In some embodiments, the patient is a mammal. In some embodiments, the patient is a pet such as a cat or a dog. In some embodiments, the patient is a human.

The terms “an inhibitor of BLM-RAD54 interaction” and “an inhibitor of BLM-RAD54 complex formation” are used interchangeably throughout the disclosure and encompass compounds that inhibit or bind to BLM, compounds that inhibit or bind to RAD54, and compounds that inhibit or disrupt the interaction or the complex formation between BLM and RAD54. In some embodiments, the term “an inhibitor or disruptor of BLM-RAD54 interaction” encompasses compounds selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, and a combination thereof. In some embodiments, the term “an inhibitor of BLM-RAD54 interaction” refers to Acetazolamide, Dipyridamole, Loxapine Succinate, or a combination thereof.

The terms “adjunct therapy”, “adjunctive therapy” or “adjuvant therapy” as used herein refer to a therapy given in addition to the main/primary treatment to maximize the effectiveness of the main/primary treatment. In some embodiments, administration of an inhibitor of BLM-RAD54 interaction can be considered as an adjunct therapy that is administered with a main/primary treatment to treat cancer.

The term “about” as used herein encompasses variations of +/−10% and more preferably +/−5%, as such variations are appropriate for practicing the present invention.

The inventor found that a stretch of 32 amino acids in BLM interact with RAD54 and enhance the chromatin remodeling function of RAD54. The inventor observed that functionally, this interaction between BLM and RAD54 increased Homologous Recombination (HR) repair resulting in decreased DNA damage in cancer cells and contributed to chemoresistance against chemotherapeutic agents such as cisplatin, camptothecin, oxaliplatin etc. promoting tumorigenesis in preclinical colon cancer mouse models. Further analysis showed increased BLM/RAD54 co-recruitment on MRP2 promoter in chemoresistant cancer cells leading to BLM-dependent enhancement of RAD54 chromatin remodeling. Based on these findings, the present disclosure provides methods for inhibiting BLM-RAD54 interaction in cancer cells, methods for treating cancer, and methods for decreasing resistance to chemotherapy in cancer cells comprising administering an inhibitor of BLM-RAD54 interaction.

In some embodiments, provided herein is a method for treating cancer in a patient in need thereof, comprising: a) administering a chemotherapy to the patient; and b) administering an inhibitor of BLM-RAD54 interaction to the patient after starting the chemotherapy or simultaneously with chemotherapy.

In some embodiments, provided herein is a method for reducing resistance to chemotherapy in a cancer patient, comprising administering an inhibitor of BLM-RAD54 interaction to the cancer patient after starting the chemotherapy or simultaneously with chemotherapy.

The findings of the inventor indicate that the administration of an inhibitor of BLM-RAD54 interaction to cancer cells reduces the level of chemoresistance exhibited by the cancer cells upon exposure to chemotherapeutic agents. That is, the administration of an inhibitor of BLM-RAD54 interaction to cancer cells helps in keeping cancer cells sensitive to chemotherapeutic agents thereby increasing effectiveness of chemotherapy. Accordingly, the methods of the present disclosure comprise administration of an inhibitor of BLM-RAD54 interaction to cancer cells after starting the chemotherapy to reduce the possibility of developing resistance to chemotherapy.

In some embodiments, the cancer treated by the methods of the present disclosure is selected from colorectal cancer, breast cancer, stomach (gastric) cancer, ovarian cancer, small cell lung cancer and leukemia. In some embodiments, the cancer treated by the methods of the present disclosure is colorectal cancer. The term “colorectal cancer” encompasses colon cancer and rectal cancer.

In some embodiments, the cancer treated by the methods of the present disclosure is the cancer where cancer cells develop resistance to primary chemotherapy due to BLM-RAD54 interaction in the cancer cells.

In some embodiments, an inhibitor of BLM-RAD54 interaction is administered as an adjunct therapy to a primary treatment for cancer, wherein the primary treatment comprises administration of one or more chemotherapeutic agents selected from (i) alkylating agents such as altretamine, bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, Trabectedin; (ii) nitrosoureas such as Carmustine, Lomustine, and Streptozocin; (iii) antimetabolites such as Azacitidine, 5-fluorouracil (5-FU), 6-mercaptopurine (6-MP), Capecitabine (Xeloda), Cladribine, Clofarabine, Cytarabine (Ara-C), Decitabine, Floxuridine, Fludarabine, Gemcitabine (Gemzar), Hydroxyurea, Methotrexate, Nelarabine, Pemetrexed (Alimta), Pentostatin, Pralatrexate, Thioguanine, Trifluridine/tipiracil combination; (iv) anti-tumor antibiotics such as Daunorubicin, Doxorubicin (Adriamycin), Epirubicin, Idarubicin, Valrubicin, Bleomycin, Dactinomycin, Mitomycin-C, Mitoxantrone; (v) Topoisomerase I inhibitors (also called camptothecins) such as Irinotecan, Topotecan; (vi) Topoisomerase II inhibitors (also called epipodophyllotoxins) such as Etoposide (VP-16), Mitoxantrone, Teniposide; (vii) mitotic inhibitors such as taxanes (e.g., Cabazitaxel, Docetaxel, Nab-paclitaxel, Paclitaxel) and vinca alkaloids (e.g., Vinblastine, Vincristine, Vinorelbine); (viii) corticosteroids such as Prednisone, Methylprednisolone, Dexamethasone; (ix) other chemotherapeutic drugs such as All-trans-retinoic acid, Arsenic trioxide, Asparaginase, Eribulin, Hydroxyurea, Ixabepilone, Mitotane, Omacetaxine, Pegaspargase, Procarbazine, Romidepsin, and Vorinostat.

In some embodiments, chemotherapy administered to the cancer patient comprises cisplatin, oxaloplatin, carboplatin, camptothecin, 5-Fluorouracil (5-FU), Capecitabine, Irinotecan, Trifluridine, tipiracil, or a combination thereof.

In some embodiments, chemotherapy administered to the cancer patient comprises cisplatin, oxaloplatin, carboplatin, camptothecin, or a combination thereof. In some embodiments, chemotherapy administered to the cancer patient comprises 5-Fluorouracil (5-FU), Capecitabine, Irinotecan, Oxaliplatin, Trifluridine, tipiracil, or a combination thereof

In some embodiments, the inhibitor of BLM-RAD54 interaction is selected from the group consisting of Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.

In some embodiments, the inhibitor of BLM-RAD54 interaction is selected from Acetazolamide or a pharmaceutically acceptable salt thereof, Dipyridamole or a pharmaceutically acceptable salt thereof, Loxapine Succinate or other pharmaceutically acceptable salt thereof, or a combination thereof.

In some embodiments, the inhibitor of BLM-RAD54 interaction is administered after starting the chemotherapy. A chemotherapy is generally administered in cycles. The inhibitor of BLM-RAD54 interaction can be administered after starting the chemotherapy. For example, the inhibitor of BLM-RAD54 interaction can be administered after completing 1 cycle, 2 cycles, 3 cycles, 4 cycles of chemotherapy and the like; or it can be administered after every cycle is completed, or after every other cycle is completed, or after every 2 cycles are completed and the like.

In some embodiments, the inhibitor of BLM-RAD54 interaction is administered simultaneously with the chemotherapy. For example, the inhibitor of BLM-RAD54 interaction is administered during the chemotherapy cycle. For example, it can be administered separately on the day of chemotherapy or it can be co-administered with the chemotherapeutic agents.

In some embodiments, the inhibitor of BLM-RAD54 interaction is administered after starting the chemotherapy, i.e., after completing a desired cycle of chemotherapy, as well as simultaneously with/during the chemotherapy.

In some embodiments, administration of the inhibitor of BLM-RAD54 interaction to a cancer patient inhibits the interaction of BLM and RAD54 in cancer cells of the patient by about 10%-80%, including values and ranges thereof, compared to levels of BLM-RAD54 interaction in the absence of administration of the inhibitor. In some embodiments, administration of the inhibitor of BLM-RAD54 interaction to a cancer patient inhibits the interaction of BLM and RAD54 in cancer cells of the patient by about 10%-75%, 10%-70%, 10%-65%, 10%-60%, 10%-50%, 10%-45%, 10%-40%, 10%-30%, 20%-80%, 20%-75%, 20%-70%, 20%-65%, 20%-60%, 20%-50%, 20%-45%, 20%-40%, 20%-35%, 30%-80%, 30%-75%, 30%-70%, 30%-65%, 30%-60%, 30%-50%, 30%-45%, 40%-80%, 40%-75%, 40%-70%, 40%-65%, 40%-60%, 40%-50%, 50%-80%, 50%-75%, 50%-70%, or 60%-80%, including values and ranges thereof, compared to levels of BLM-RAD54 interaction in the absence of the inhibitor. In some embodiments, administration of the inhibitor of BLM-RAD54 interaction to a cancer patient inhibits the interaction of BLM and RAD54 in cancer cells of the patient by about 10%, 15%, 20%, 25%, 30%, 35%, 40%, 45%, 50%, 55%, 60%, 65%, 70%, 75%, or 80%, compared to levels of BLM-RAD54 interaction in the absence of the inhibitor.

In some embodiments, administration of the inhibitor of BLM-RAD54 interaction to a cancer patient reduces proliferation of cancer cells by about 10%-80%, including values and ranges thereof, compared to proliferation of cancer cells in the absence of the inhibitor. In some embodiments, administration of the inhibitor of BLM-RAD54 interaction to a cancer patient reduces proliferation of cancer cells by about 10%-75%, 10%-70%, 10%-65%, 10%-60%, 10%-50%, 10%-45%, 10%-40%, 10%-30%, 20%-80%, 20%-75%, 20%-70%, 20%-65%, 20%-60%, 20%-50%, 20%-45%, 20%-40%, 20%-35%, 30%-80%, 30%-75%, 30%-70%, 30%-65%, 30%-60%, 30%-50%, 30%-45%, 40%-80%, 40%-75%, 40%-70%, 40%-65%, 40%-60%, 40%-50%, 50%-80%, 50%-75%, 50%-70%, or 60%-80%, including values and ranges thereof, compared to proliferation of cancer cells in the absence of the inhibitor. In some embodiments, administration of the inhibitor of BLM-RAD54 interaction to a cancer patient reduces proliferation of cancer cells by about 10%, 15%, 20%, 25%, 30%, 35%, 40%, 45%, 50%, 55%, 60%, 65%, 70%, 75%, or 80%, compared to proliferation of cancer cells in the absence of the inhibitor.

The dosage amount and the frequency of administration of the inhibitor of BLM-RAD54 interaction to the patient varies based on a variety of factors, such as, age, weight, sex, a medical condition of the patient, and/or the dosage and frequency of chemotherapy administered to the patient. Chemotherapy is often administered in cycles, i.e., there is a period of treatment and a period of rest, and this cycle is repeated. The inhibitor of BLM-RAD54 interaction according to the present methods can be administered after the first exposure of cancer cells to chemotherapy, after the subsequent exposures of cancer cells to chemotherapy, after every exposure of cancer cells to chemotherapy, after every other exposure of cancer cells to chemotherapy, after every second exposure of cancer cells to chemotherapy and the like. The frequency of administration of the inhibitor of BLM-RAD54 interaction can vary to ensure that a majority of cancer cells, such as about 25%, 30%, 35%, 40%, 45%, 50%, 55%, 60%, 65%, 70%, 75%, 80%, 90%, 95%, or 99% of cancer cells remain sensitive to chemotherapy.

In some embodiments, the inhibitor of BLM-RAD54 interaction is administered orally or parenterally. Parenteral administration comprises administration via injection or infusion. In some embodiments, parenteral administration is selected from intravenous, intramuscular, intradermal, subcutaneous, intratumoral, intralesional, intraperitoneal, and intrathecal administration. In some embodiments, parenteral administration is administration via intravenous infusion.

In some embodiments, acetazolamide or a pharmaceutically acceptable salt thereof is administered to a subject receiving chemotherapy in an amount of about 100 mg per day to 1500 mg per day, including values and ranges thereof, such as about 200-1500 mg, 200-1300 mg, 200-1000 mg, 200-800 mg, 200-700 mg, 200-500 mg, 200-400 mg, 250-1200 mg, 250-1000 mg, 250-800 mg, 250-750 mg, 250-650 mg, 250-550 mg, 250-500 mg, 250-450 mg, 300-1500 mg, 300-1200 mg, 300-1000 mg, 300-800 mg, 300-750 mg, 300-600 mg, 300-500 mg, 400-1500 mg, 400-1200 mg, 400-1000 mg, 400-800 mg, 400-600 mg, 500-1500 mg, 500-1200 mg, 500-1000 mg, 500-800 mg, 500-750 mg, 700-1500 mg, 700-1300 mg, 700-1200 mg, 700-1000 mg, 900-1300 mg, or 900-1500 mg per day. In some embodiments, acetazolamide or a pharmaceutically acceptable salt thereof is administered to a subject receiving chemotherapy in an amount of about 250 mg to about 1000 mg per day or about 250 mg to about 500 mg per day. The daily dose can be administered once or multiple times in a day. The frequency of administration may vary based on various factors such as age, weight, sex, and dosing and frequency of primary chemotherapeutic agents being administered. In some embodiments, acetazolamide or a pharmaceutically acceptable salt thereof is administered to a subject receiving chemotherapy orally or parenterally.

In some embodiments, dipyridamole or a pharmaceutically acceptable salt thereof is administered to a subject receiving chemotherapy in an amount of about 200-600 mg per day, including values and ranges thereof, such as about 200-500 mg, 200-400 mg, 250-600 mg, 250-500 mg, 250-400 mg, 300-600 mg, 300-500 mg, 300-400 mg, 400-600 mg, or about 400-500 mg per day. In some embodiments, dipyridamole or a pharmaceutically acceptable salt thereof is administered to a subject receiving chemotherapy in an amount of about 300-400 mg per day. The daily dose can be administered once or multiple times in a day. The frequency of administration may vary based on various factors such as age, weight, sex, and dosing and frequency of primary chemotherapeutic agents being administered. In some embodiments, dipyridamole or a pharmaceutically acceptable salt thereof is administered to a subject receiving chemotherapy orally or parenterally.

In some embodiments, loxapine succinate or other pharmaceutically acceptable salt of loxapine is administered to a subject receiving chemotherapy in an amount of about 10-250 mg per day, including values and ranges thereof, such as about 10-200 mg, 10-150 mg, 10-100 mg, 10-75 mg, 10-60 mg, 10-50 mg, 10-40 mg, 25-250 mg, 25-200 mg, 25-150 mg, 25-100 mg, 25-75 mg, 25-50 mg, 50-250 mg, 50-200 mg, 50-150 mg, 50-100 mg, 60-200 mg, 60-150 mg, 60-100 mg, 75-250 mg, 75-200 mg, 75-150 mg, 75-100 mg, 100-250 mg, or 100-200 mg per day. In some embodiments, loxapine succinate or other pharmaceutically acceptable salt of loxapine is administered to a subject receiving chemotherapy in an amount of about 10-60 mg per day. The daily dose can be administered once or multiple times in a day. The frequency of administration can vary based on various factors such as age, weight, sex, and dosing and frequency of primary chemotherapeutic agents being administered. In some embodiments, loxapine succinate or other pharmaceutically acceptable salt of loxapine is administered to a subject receiving chemotherapy orally or parenterally.

The present disclosure also provides a method for inhibiting an interaction of BLM and RAD54 in cancer cells, comprising contacting the cancer cells with the inhibitor of BLM-RAD54 interaction as described herein. In some embodiments, the cancer cells are colorectal cancer cells which include colon cancer cells and/or rectal cancer cells. When cancer cells are contacted with the inhibitor of BLM-RAD54 interaction, the inhibitor reduces the interaction of BLM and RAD54 or disrupts the interaction between BLM and RAD54 in cancer cells by the values and ranges described herein compared to untreated or control-treated cancer cells. When cancer cells are contacted with the inhibitor of BLM-RAD54 interaction, the inhibitor reduces the proliferation of cancer cells by the values and ranges described herein compared to untreated or control-treated cancer cells.

In some embodiments, the present disclosure provides an inhibitor of BLM-RAD54 interaction for use as an adjunct therapy in the treatment of cancer. In some embodiments, the inhibitor of BLM-RAD54 interaction is used as an adjunct therapy for treating a cancer selected from colorectal cancer, breast cancer, stomach (gastric) cancer, ovarian cancer, small cell lung cancer and leukemia. In some embodiments, the inhibitor of BLM-RAD54 interaction is used as an adjunct therapy for treating colorectal cancer which encompasses colon cancer and rectal cancer. In some embodiments, the inhibitor of BLM-RAD54 interaction is used as an adjunct therapy for treating colon cancer.

An inhibitor of BLM-RAD54 interaction is used as an adjunct therapy to increase the effectiveness of the main/primary treatment for cancer. In some embodiments, an inhibitor of BLM-RAD54 interaction is used as an adjunct therapy to a primary treatment for cancer, wherein the primary treatment comprises administration of one or more chemotherapeutic agents selected from (i) alkylating agents such as altretamine, bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, Trabectedin; (ii) nitrosoureas such as Carmustine, Lomustine, and Streptozocin; (iii) antimetabolites such as Azacitidine, 5-fluorouracil (5-FU), 6-mercaptopurine (6-MP), Capecitabine (Xeloda), Cladribine, Clofarabine, Cytarabine (Ara-C), Decitabine, Floxuridine, Fludarabine, Gemcitabine (Gemzar), Hydroxyurea, Methotrexate, Nelarabine, Pemetrexed (Alimta), Pentostatin, Pralatrexate, Thioguanine, Trifluridine/tipiracil combination; (iv) anti-tumor antibiotics such as Daunorubicin, Doxorubicin (Adriamycin), Epirubicin, Idarubicin, Valrubicin, Bleomycin, Dactinomycin, Mitomycin-C, Mitoxantrone; (v) Topoisomerase I inhibitors (also called camptothecins) such as Irinotecan, Topotecan; (vi) Topoisomerase II inhibitors (also called epipodophyllotoxins) such as Etoposide (VP-16), Mitoxantrone, Teniposide; (vii) mitotic inhibitors such as taxanes (e.g., Cabazitaxel, Docetaxel, Nab-paclitaxel, Paclitaxel) and vinca alkaloids (e.g., Vinblastine, Vincristine, Vinorelbine); (viii) corticosteroids such as Prednisone, Methylprednisolone, Dexamethasone; (ix) other chemotherapeutic drugs such as All-trans-retinoic acid, Arsenic trioxide, Asparaginase, Eribulin, Hydroxyurea, Ixabepilone, Mitotane, Omacetaxine, Pegaspargase, Procarbazine, Romidepsin, and Vorinostat.

In some embodiments, an inhibitor of BLM-RAD54 interaction is used as an adjunct therapy to a primary treatment for cancer, wherein the primary treatment comprises administration of a chemotherapeutic agent selected from cisplatin, oxaloplatin, carboplatin, camptothecin, 5-Fluorouracil (5-FU), Capecitabine, Irinotecan, Trifluridine, tipiracil, or a combination thereof.

In some embodiments, an inhibitor of BLM-RAD54 interaction is used as an adjunct therapy to a primary treatment for cancer, wherein the primary treatment comprises administration of a chemotherapeutic agent selected from cisplatin, oxaloplatin, carboplatin, camptothecin, or a combination thereof.

In some embodiments, an inhibitor of BLM-RAD54 interaction is used as an adjunct therapy to a primary treatment for cancer, wherein the primary treatment comprises administration of a chemotherapeutic agent selected from 5-Fluorouracil (5-FU), Capecitabine, Irinotecan, Oxaliplatin, Trifluridine, tipiracil, or a combination thereof.

In some embodiments, an inhibitor of BLM-RAD54 interaction is used as an adjunct therapy to a primary treatment for cancer, wherein the primary treatment comprises administration of a chemotherapeutic agent selected from 5-Fluorouracil (5-FU), Capecitabine, Irinotecan, Oxaliplatin, Trifluridine, and tipiracil.

In some embodiments, an inhibitor of BLM-RAD54 interaction is used as an adjunct therapy to a primary treatment for cancer, wherein the cancer exhibits resistance to the primary treatment due to BLM-RAD54 interaction in cancer cells.

Provided herein is an inhibitor of BLM-RAD54 interaction for use in a method of treating cancer, wherein the inhibitor of BLM-RAD54 interaction is an adjunct therapy. In some embodiments, the method of treating cancer comprises the administration of one or more chemotherapeutic agents selected from (i) alkylating agents such as altretamine, bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, Trabectedin; (ii) nitrosoureas such as Carmustine, Lomustine, and Streptozocin; (iii) antimetabolites such as Azacitidine, 5-fluorouracil (5-FU), 6-mercaptopurine (6-MP), Capecitabine (Xeloda), Cladribine, Clofarabine, Cytarabine (Ara-C), Decitabine, Floxuridine, Fludarabine, Gemcitabine (Gemzar), Hydroxyurea, Methotrexate, Nelarabine, Pemetrexed (Alimta), Pentostatin, Pralatrexate, Thioguanine, Trifluridine/tipiracil combination; (iv) anti-tumor antibiotics such as Daunorubicin, Doxorubicin (Adriamycin), Epirubicin, Idarubicin, Valrubicin, Bleomycin, Dactinomycin, Mitomycin-C, Mitoxantrone; (v) Topoisomerase I inhibitors (also called camptothecins) such as Irinotecan, Topotecan; (vi) Topoisomerase II inhibitors (also called epipodophyllotoxins) such as Etoposide (VP-16), Mitoxantrone, Teniposide; (vii) mitotic inhibitors such as taxanes (e.g., Cabazitaxel, Docetaxel, Nab-paclitaxel, Paclitaxel) and vinca alkaloids (e.g., Vinblastine, Vincristine, Vinorelbine); (viii) corticosteroids such as Prednisone, Methylprednisolone, Dexamethasone; (ix) other chemotherapeutic drugs such as All-trans-retinoic acid, Arsenic trioxide, Asparaginase, Eribulin, Hydroxyurea, Ixabepilone, Mitotane, Omacetaxine, Pegaspargase, Procarbazine, Romidepsin, and Vorinostat. In some embodiments, the method of treating cancer comprises the administration of a chemotherapeutic agent selected from cisplatin, oxaloplatin, carboplatin, camptothecin, or a combination thereof. In some embodiments, the method of treating cancer comprises the administration of a chemotherapeutic agent selected from 5-Fluorouracil (5-FU), Capecitabine, Irinotecan, Oxaliplatin, Trifluridine, tipiracil, or a combination thereof. In some embodiments, the method of treating cancer comprises the administration of 5-Fluorouracil (5-FU), Capecitabine, Irinotecan, Oxaliplatin, Trifluridine, and tipiracil.

In some embodiments, the present disclosure provides an inhibitor of BLM-RAD54 interaction for use in reducing resistance to chemotherapy in a cancer patient. In some embodiments, the inhibitor of BLM-RAD54 interaction reduces resistance to chemotherapy in a patient having a cancer selected from colorectal cancer, breast cancer, stomach (gastric) cancer, ovarian cancer, small cell lung cancer and leukemia. In some embodiments, the inhibitor of BLM-RAD54 interaction reduces resistance to chemotherapy in a colorectal cancer patient. The colorectal cancer can be colon cancer or rectal cancer. In some embodiments, the inhibitor of BLM-RAD54 interaction reduces resistance to chemotherapy in a colon cancer patient.

In some embodiments, the present disclosure provides an inhibitor of BLM-RAD54 interaction for use in reducing resistance to chemotherapy in a cancer patient, wherein the chemotherapy comprises administration of one or more chemotherapeutic agents selected from (i) alkylating agents such as altretamine, bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, Trabectedin; (ii) nitrosoureas such as Carmustine, Lomustine, and Streptozocin; (iii) antimetabolites such as Azacitidine, 5-fluorouracil (5-FU), 6-mercaptopurine (6-MP), Capecitabine (Xeloda), Cladribine, Clofarabine, Cytarabine (Ara-C), Decitabine, Floxuridine, Fludarabine, Gemcitabine (Gemzar), Hydroxyurea, Methotrexate, Nelarabine, Pemetrexed (Alimta), Pentostatin, Pralatrexate, Thioguanine, Trifluridine/tipiracil combination; (iv) anti-tumor antibiotics such as Daunorubicin, Doxorubicin (Adriamycin), Epirubicin, Idarubicin, Valrubicin, Bleomycin, Dactinomycin, Mitomycin-C, Mitoxantrone; (v) Topoisomerase I inhibitors (also called camptothecins) such as Irinotecan, Topotecan; (vi) Topoisomerase II inhibitors (also called epipodophyllotoxins) such as Etoposide (VP-16), Mitoxantrone, Teniposide; (vii) mitotic inhibitors such as taxanes (e.g., Cabazitaxel, Docetaxel, Nab-paclitaxel, Paclitaxel) and vinca alkaloids (e.g., Vinblastine, Vincristine, Vinorelbine); (viii) corticosteroids such as Prednisone, Methylprednisolone, Dexamethasone; (ix) other chemotherapeutic drugs such as All-trans-retinoic acid, Arsenic trioxide, Asparaginase, Eribulin, Hydroxyurea, Ixabepilone, Mitotane, Omacetaxine, Pegaspargase, Procarbazine, Romidepsin, and Vorinostat.

In some embodiments, the present disclosure provides an inhibitor of BLM-RAD54 interaction for use in reducing resistance to chemotherapy in a cancer patient, wherein the chemotherapy comprises administration of cisplatin, oxaloplatin, carboplatin, camptothecin, 5-Fluorouracil (5-FU), Capecitabine, Irinotecan, Oxaliplatin, Trifluridine, tipiracil, or a combination thereof.

In some embodiments, the present disclosure provides an inhibitor of BLM-RAD54 interaction for use in reducing resistance to chemotherapy in a cancer patient, wherein the chemotherapy comprises administration of cisplatin, oxaloplatin, carboplatin, or camptothecin, or a combination thereof.

In some embodiments, the present disclosure provides an inhibitor of BLM-RAD54 interaction for use in reducing resistance to chemotherapy in a cancer patient, wherein the chemotherapy comprises administration of 5-Fluorouracil (5-FU), Capecitabine, Irinotecan, Oxaliplatin, Trifluridine, tipiracil, or a combination thereof.

In some embodiments, the present disclosure provides an inhibitor of BLM-RAD54 interaction for use in reducing resistance to chemotherapy in a cancer patient, wherein the chemotherapy comprises administration of 5-Fluorouracil (5-FU), Capecitabine, Irinotecan, Oxaliplatin, Trifluridine, and tipiracil.

Provided herein is an inhibitor of BLM-RAD54 interaction for use in a method of treating cancer. In some embodiments, the method of treating cancer comprises the administration of one or more chemotherapeutic agents selected from (i) alkylating agents such as altretamine, bendamustine, Busulfan, Carboplatin, Carmustine, Chlorambucil, Cisplatin, Cyclophosphamide, Dacarbazine, Ifosfamide, Lomustine, Mechlorethamine, Melphalan, Oxaliplatin, Temozolomide, Thiotepa, Trabectedin; (ii) nitrosoureas such as Carmustine, Lomustine, and Streptozocin; (iii) antimetabolites such as Azacitidine, 5-fluorouracil (5-FU), 6-mercaptopurine (6-MP), Capecitabine (Xeloda), Cladribine, Clofarabine, Cytarabine (Ara-C), Decitabine, Floxuridine, Fludarabine, Gemcitabine (Gemzar), Hydroxyurea, Methotrexate, Nelarabine, Pemetrexed (Alimta), Pentostatin, Pralatrexate, Thioguanine, Trifluridine/tipiracil combination; (iv) anti-tumor antibiotics such as Daunorubicin, Doxorubicin (Adriamycin), Epirubicin, Idarubicin, Valrubicin, Bleomycin, Dactinomycin, Mitomycin-C, Mitoxantrone; (v) Topoisomerase I inhibitors (also called camptothecins) such as Irinotecan, Topotecan; (vi) Topoisomerase II inhibitors (also called epipodophyllotoxins) such as Etoposide (VP-16), Mitoxantrone, Teniposide; (vii) mitotic inhibitors such as taxanes (e.g., Cabazitaxel, Docetaxel, Nab-paclitaxel, Paclitaxel) and vinca alkaloids (e.g., Vinblastine, Vincristine, Vinorelbine); (viii) corticosteroids such as Prednisone, Methylprednisolone, Dexamethasone; (ix) other chemotherapeutic drugs such as All-trans-retinoic acid, Arsenic trioxide, Asparaginase, Eribulin, Hydroxyurea, Ixabepilone, Mitotane, Omacetaxine, Pegaspargase, Procarbazine, Romidepsin, and Vorinostat.

Provided herein is an inhibitor of BLM-RAD54 interaction for use in a method of treating cancer, wherein the method of treating cancer comprises administration of a chemotherapeutic agent selected from cisplatin, oxaloplatin, carboplatin, camptothecin, 5-Fluorouracil (5-FU), Capecitabine, Irinotecan, Trifluridine, tipiracil, or a combination thereof.

Provided herein is an inhibitor of BLM-RAD54 interaction for use in a method of treating cancer, wherein the method of treating cancer comprises administration of a chemotherapeutic agent selected from cisplatin, oxaloplatin, carboplatin, camptothecin, or a combination thereof.

Provided herein is an inhibitor of BLM-RAD54 interaction for use in a method of treating cancer, wherein the method of treating cancer comprises administration of a chemotherapeutic agent selected from 5-Fluorouracil (5-FU), Capecitabine, Irinotecan, Oxaliplatin, Trifluridine, tipiracil, or a combination thereof.

Provided herein is an inhibitor of BLM-RAD54 interaction for use in a method of treating cancer, wherein the method of treating cancer comprises administration of 5-Fluorouracil (5-FU), Capecitabine, Irinotecan, Oxaliplatin, Trifluridine, and tipiracil.

In some embodiments, the inhibitor of BLM-RAD54 interaction is selected from the group consisting of Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.

In some embodiments, the inhibitor of BLM-RAD54 interaction is selected from Acetazolamide or a pharmaceutically acceptable salt thereof, Dipyridamole or a pharmaceutically acceptable salt thereof, Loxapine Succinate or other pharmaceutically acceptable salt thereof, or a combination thereof.

In some embodiments, the inhibitor of BLM-RAD54 interaction for use in a method for treating cancer inhibits the interaction of BLM and RAD54 in cancer cells of the patient by about 10%-80%, 10%-75%, 10%-70%, 10%-65%, 10%-60%, 10%-50%, 10%-45%, 10%-40%, 10%-30%, 20%-80%, 20%-75%, 20%-70%, 20%-65%, 20%-60%, 20%-50%, 20%-45%, 20%-40%, 20%-35%, 30%-80%, 30%-75%, 30%-70%, 30%-65%, 30%-60%, 30%-50%, 30%-45%, 40%-80%, 40%-75%, 40%-70%, 40%-65%, 40%-60%, 40%-50%, 50%-80%, 50%-75%, 50%-70%, or 60%-80%, including values and ranges thereof, compared to levels of BLM-RAD54 interaction in the absence of the inhibitor. In some embodiments, the inhibitor of BLM-RAD54 interaction for use in a method for treating cancer inhibits the interaction of BLM and RAD54 in cancer cells of the patient by about 10%, 15%, 20%, 25%, 30%, 35%, 40%, 45%, 50%, 55%, 60%, 65%, 70%, 75%, or 80%, compared to levels of BLM-RAD54 interaction in the absence of the inhibitor.

In some embodiments, the inhibitor of BLM-RAD54 interaction for use in a method for treating cancer reduces proliferation of cancer cells by about 10%-80%, 10%-75%, 10%-70%, 10%-65%, 10%-60%, 10%-50%, 10%-45%, 10%-40%, 10%-30%, 20%-80%, 20%-75%, 20%-70%, 20%-65%, 20%-60%, 20%-50%, 20%-45%, 20%-40%, 20%-35%, 30%-80%, 30%-75%, 30%-70%, 30%-65%, 30%-60%, 30%-50%, 30%-45%, 40%-80%, 40%-75%, 40%-70%, 40%-65%, 40%-60%, 40%-50%, 50%-80%, 50%-75%, 50%-70%, or 60%-80%, including values and ranges thereof, compared to proliferation of cancer cells in the absence of the inhibitor. In some embodiments, the inhibitor of BLM-RAD54 interaction for use in a method for treating cancer reduces proliferation of cancer cells by about 10%, 15%, 20%, 25%, 30%, 35%, 40%, 45%, 50%, 55%, 60%, 65%, 70%, 75%, or 80%, compared to proliferation of cancer cells in the absence of the inhibitor.

The dosing, the frequency of administration, and the routes of administration of the inhibitor of BLM-RAD54 interaction are described above.

The present disclosure also provides use of an inhibitor of BLM-RAD54 interaction as an adjunct therapy in the treatment of cancer. An inhibitor of BLM-RAD54 interaction is used as an adjunct therapy to a primary treatment as described above. The present disclosure also provides use of an inhibitor of BLM-RAD54 interaction for reducing resistance to chemotherapy in the treatment of cancer. The inhibitors of BLM-RAD54 interaction, their dosing, frequency and routes of administration and the cancers in which the inhibitors of BLM-RAD54 interaction are employed are as described above.

Descriptions of well-known/conventional methods/steps and techniques are omitted so as to not unnecessarily obscure the embodiments herein. Further, the disclosure herein provides for examples illustrating the above-described embodiments, and in order to illustrate the embodiments of the present disclosure certain aspects have been employed. The examples used herein for such illustration are intended merely to facilitate an understanding of ways in which the embodiments herein may be practiced and to further enable those of skill in the art to practice the embodiments herein. Accordingly, the following examples should not be construed as limiting the scope of the embodiments herein.

### EXAMPLES

### Example 1: BLM-RAD54 Interaction is Enhanced in Colon Cancer Cells

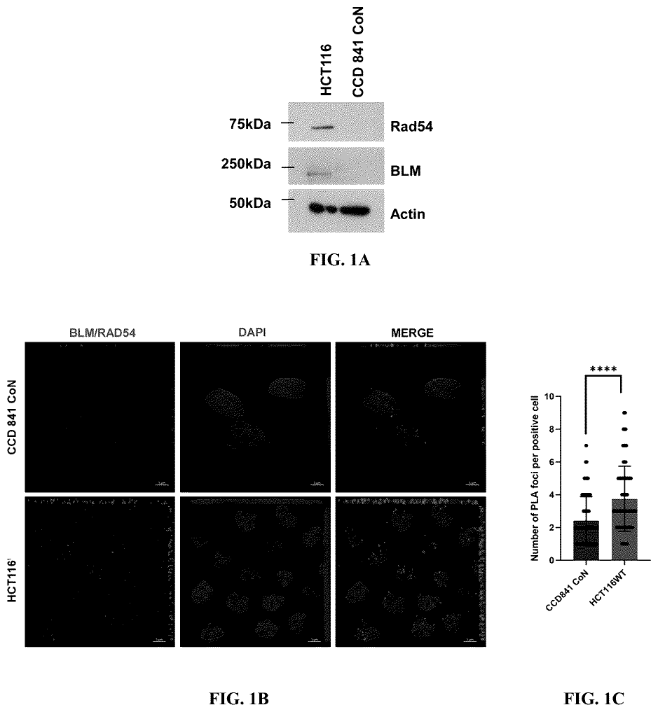

Whole cell lysates were prepared from colon cancer cells (HCT116) and normal colon epithelial cells (CCD 841 CoN). Immunoblotting was carried out with the BLM and RAD54 antibodies. The experiment was repeated three times. The results are shown in FIG. 1A.

Proximity Ligation Assay (PLA) was carried out with antibodies to BLM and RAD54. Amplified signals post DNA polymerization and hybridization of the complementary oligonucleotides labelled with fluorogenic readout were visualized in the Red channel. DAPI was visualized in the Blue channel while Merge indicates the combination of the Red and Blue channels. The Proximity Ligation Assay results in FIG. 1B indicate greater interaction between BLM and RAD54 in cancer cells. The data from FIG. 1B was quantified and is shown in FIG. 1C. 50 cells were counted for each cell line. Mean±SD.

### Example 2: BLM (181-212) Enhanced RAD54-Dependent Chromatin Remodeling

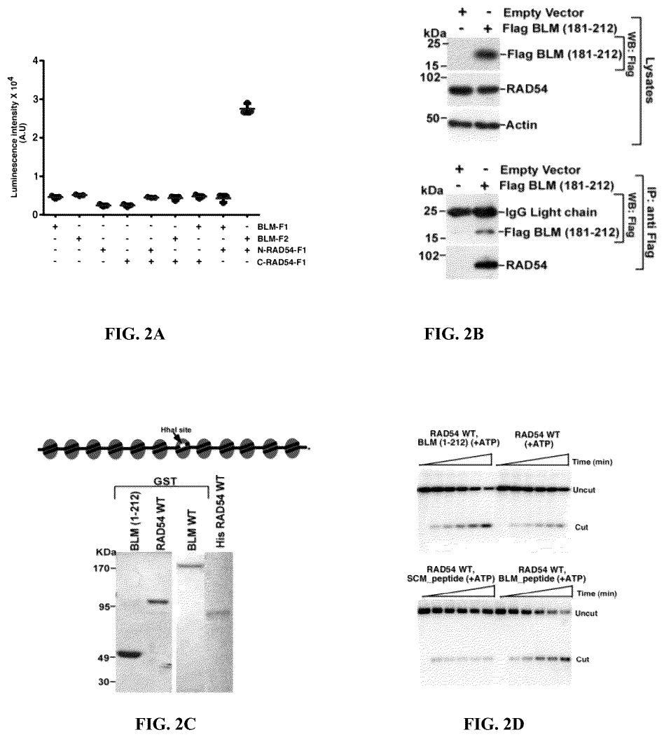

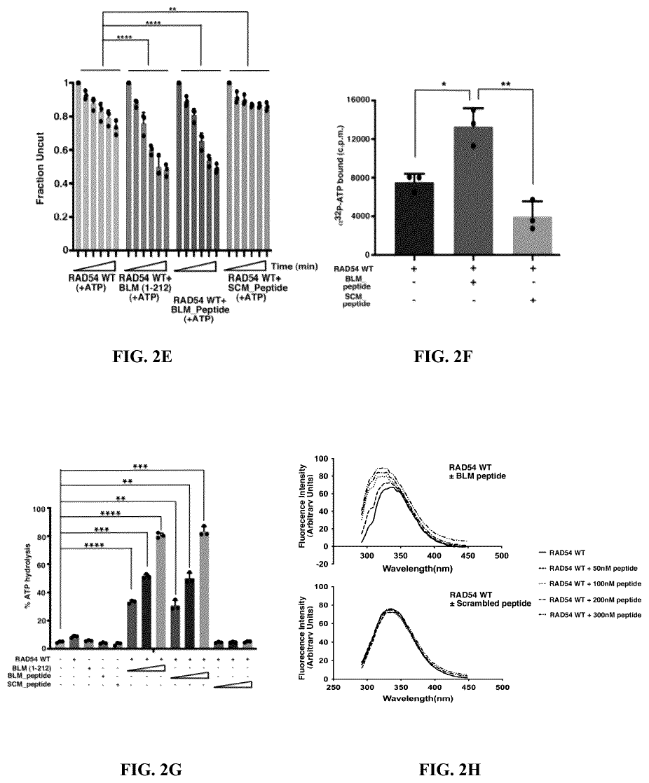

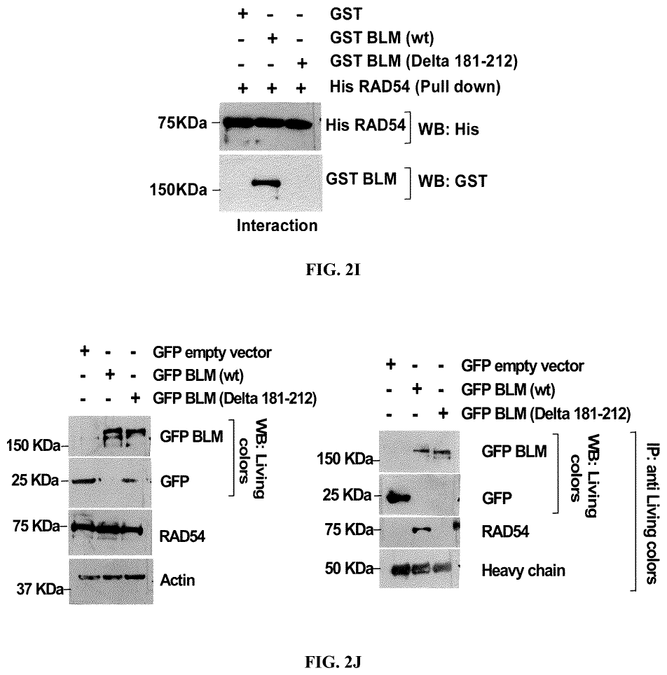

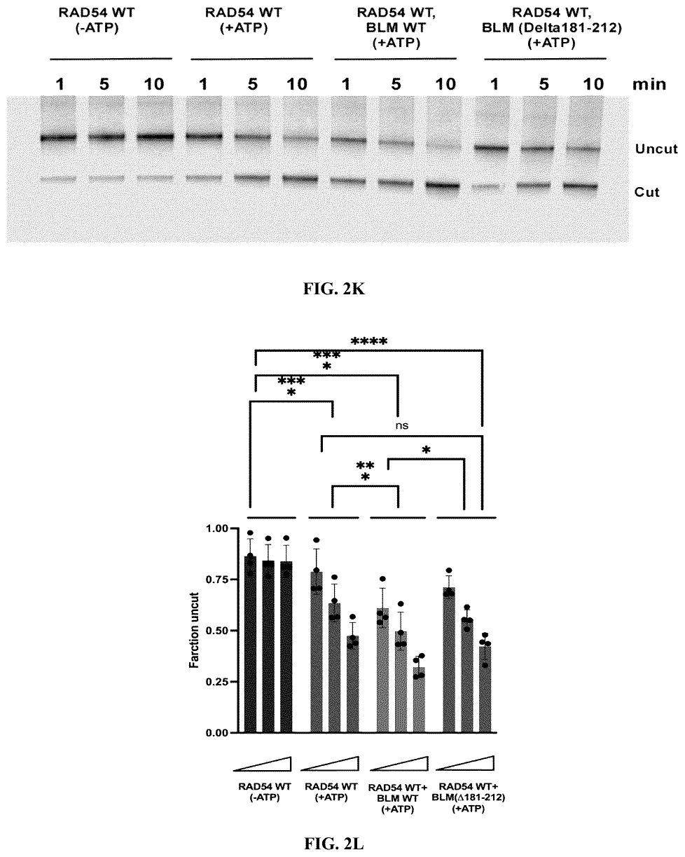

It has been earlier demonstrated that the N-terminal region of BLM (1-212) (the UniProt identifier for the full-length sequence of human BLM is P54132) enhanced the RAD54-mediated chromatin remodeling activity (Srivastava V, Modi P, Tripathi V, Mudgal R, De S, and Sengupta S. BLM helicase stimulates the ATPase and chromatin-remodeling activities of RAD54. J Cell Sci. 2009; 122(Pt 17):3093-103). Using a Renilla luciferase-based protein complementation assay (PCA) (Stefan E, Aquin S, Berger N, Landry C R, Nyfeler B, Bouvier M, et al. Quantification of dynamic protein complexes using Renilla luciferase fragment complementation applied to protein kinase A activities in vivo. Proc Natl Acad Sci USA. 2007; 104(43):16916-21.), the inventors observed that BLM (181-212) cloned to luciferase fragment (BLM-F2) was sufficient to interact with the N-terminal region of RAD54 (1-212, N-RAD54-F1) (FIG. 2A) (the UniProt identifier for the full-length sequence of human RAD54 is Q92698). Specifically, N-RAD54-F1, C-RAD54-F1, BLM-F1 and BLM-F2 were transfected in HEK293T. Forty-eight hours post-transfection, Renilla luciferase-based PCA were carried out with the indicated combination of the expressed proteins. The experiment was repeated three times. Mean±S.D.

Immunoprecipitations done in HCT116 BLM−/− cells expressing Flag tagged NLS BLM (181-212) demonstrated in cellulo interaction between this peptide stretch in BLM and endogenous RAD54 (FIG. 2B). For this experiment, HCT116 BLM−/− cells were transfected with p3×Flag-Myc-CMV24 BLM (181-212), or the empty vector and lysates were made. Flag tagged BLM (181-212) was immunoprecipitated using anti-Flag beads and the immunoprecipitate probed for RAD54. The experiment was repeated three times. One representative experiment has been shown in FIG. 2B.

To determine whether BLM (181-212) was sufficient to enhance RAD54 mediated chromatin remodeling, measurement of the chromatin remodeling activity was carried out by using a restriction enzyme accessibility (REA) assay on chromatinized G5E4 array. G5E4 array containing 12 nucleosomes with a centrally located HhaI site occluded by one nucleosome is shown at the top of the gel picture in FIG. 2C. (Neely K E, Hassan A H, Wallberg A E, Steger D J, Cairns B R, Wright A P, et al. Activation domain-mediated targeting of the SWI/SNF complex to promoters stimulates transcription from nucleosome arrays. Mol Cell. 1999; 4(4):649-55). Coomassie gels indicating the purified GST BLM (1-212), GST RAD54 (1-747) (WT), GST BLM (1-1417) (WT), His RAD54 (1-747) (WT) are shown in FIG. 2C.

REA assays were carried out with chromatinized G5E4 array using recombinant RAD54, BLM (1-212), BLM peptide (181-212) (termed BLM_peptide) or a scrambled peptide (termed SCM_peptide) having the same amino acid composition. All reactions were carried out in the presence of ATP. The reactions were stopped after 0 minute, 2 minutes, 4 minutes, 6 minutes, 8 minutes and 10 minutes. The fraction uncut is presented. The data is from three independent experiments. Mean±S.D. The gel pictures are shown in FIG. 2D. FIG. 2E shows the quantitation of the data in FIG. 2D. It was observed that only BLM_peptide could enhance the chromatin remodeling activity of RAD54, to the same extent as BLM (1-212).

BLM_peptide, i.e., BLM (181-212), carries out this function by enhancing the binding of ATP to RAD54 as shown in FIG. 2F. The quantitation of the ATP binding assay was carried out using RAD54 WT, RAD54 WT+BLM_peptide, and RAD54 WT+SCM_peptide. The data is from three independent experiments. Mean±S.D.

BLM_peptide enhanced the binding of ATP to RAD54 and increased the ATPase activity of RAD54 leading to increased ATP hydrolysis as shown in FIG. 2G. FIG. 2G shows the quantitation of the ATPase activity carried out using RAD54 WT, BLM (1-212), BLM_peptide, SCM_peptide, RAD54 WT+a gradient of BLM (1-212) (180 nM, 360 nM, 540 nM), RAD54 WT+a gradient of BLM_peptide (180 nM, 360 nM, 540 nM), RAD54 WT+a gradient of SCM_peptide (180 nM, 360 nM, 540 nM). The data is from three independent experiments. Mean±S.D.

To determine how BLM peptide affects ATP binding and hydrolysis, tryptophan fluorescence assays were carried out using full-length recombinant RAD54 in presence of either BLM_peptide or SCM_peptide (FIG. 2H). Tryptophan fluorescence assays were carried out with RAD54 WT alone or RAD54 WT in presence of the indicated concentrations of either BLM_peptide (upper panel) or SCM_peptide (lower panel). The experiment was repeated three times and one representative experiment has been shown. Increasing amount of BLM_peptide led to progressively enhanced fluorescence quenching, thereby indicating that BLM alters the conformation of RAD54 by interacting via the internal 32 amino acids (181-212).

Next, it was tested whether removal of amino acids (181-212) in BLM abrogates the interaction between BLM and RAD54. For this, in vitro interaction was carried out between the bound GST or GST-BLM wildtype or GST BLM (Delta 181-212) and soluble His RAD54. The bound and interacting proteins were detected by anti-GST and anti-His antibodies, respectively. The experiment was repeated three times. See FIG. 2I.

In vivo interactions were carried out between GFP alone, GFP BLM or GFP BLM (Delta 181-212). FIG. 2J, left panel, shows the expression of the respective proteins. Immunoblotting was carried out with the indicated antibodies. Immunoprecipitation (IP) was carried out with anti-GFP (Living colours) antibody and interacting proteins were detected with anti-RAD54 antibody. See FIG. 2J, right panel. The experiment was repeated three times.

The data in FIGS. 2I and 2J show that the lack of BLM (181-212) leads to complete loss of interaction between BLM and RAD54. That is, the 32 amino acids in BLM (181-212) mediates the interaction between BLM and RAD54.

Next, it was determined whether the lack of amino acids (181-212) in BLM abrogates the enhancement of RAD54 mediated chromatin remodeling.

For this, chromatin remodeling assays were carried out under the following conditions: RAD54 wildtype alone (−ATP), RAD54 wildtype alone (+ATP), RAD54 wildtype+BLM wildtype (+ATP), RAD54 wildtype+BLM (Delta 181-212) (+ATP). A typical chromatin remodeling assay is shown in FIG. 2K. FIG. 2L shows the quantitation of the data in FIG. 2K. The experiment was repeated three times. Mean±SD.

### Example 3: Interaction of BLM with RAD54 Enhanced Cell Proliferation

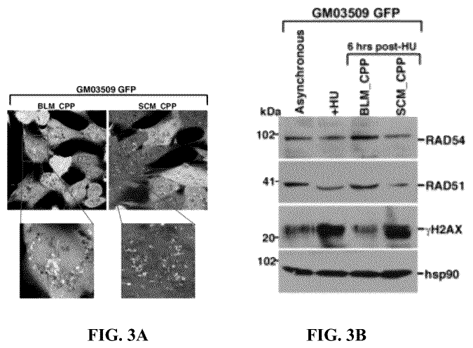

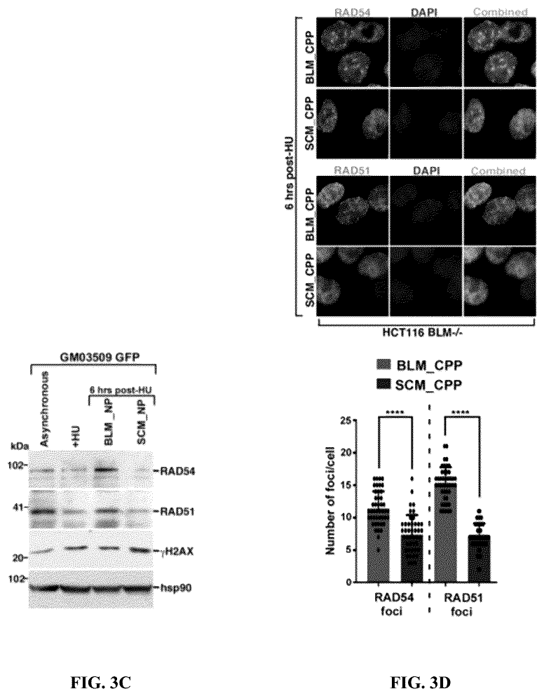

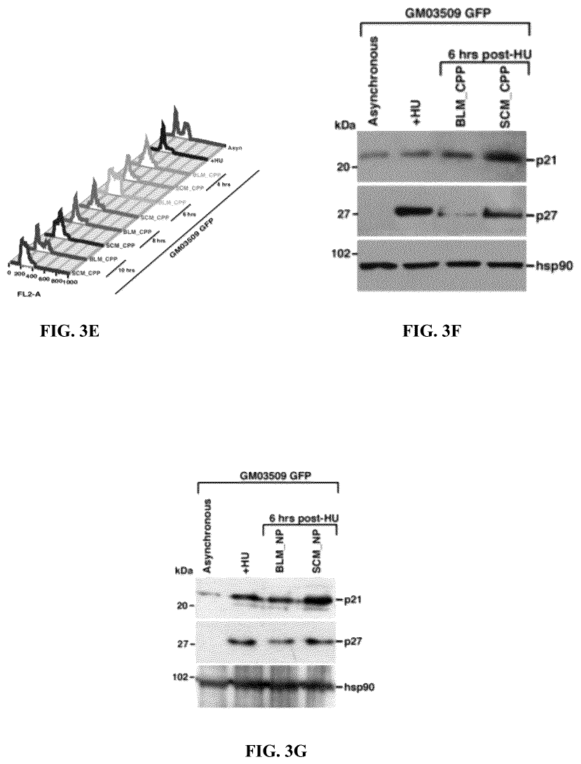

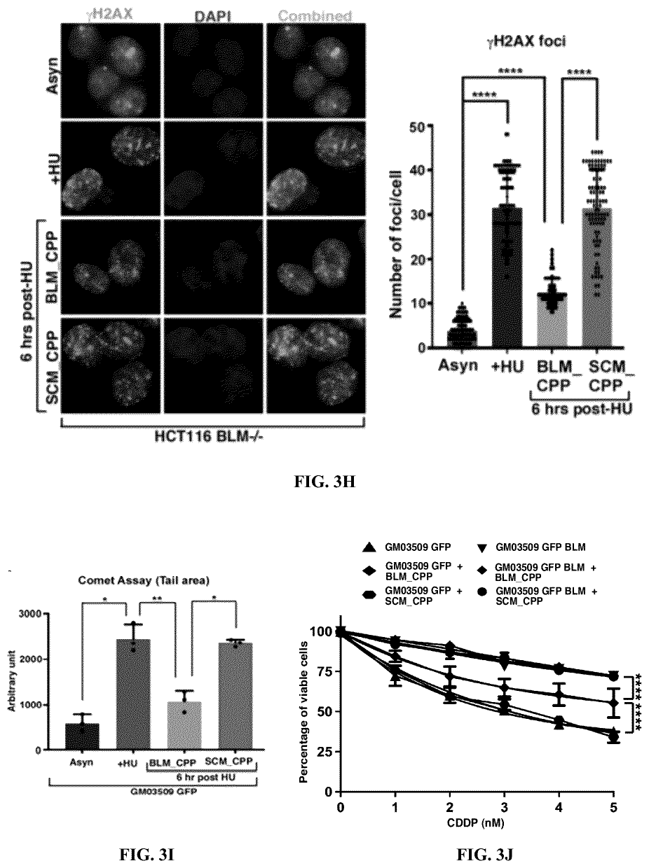

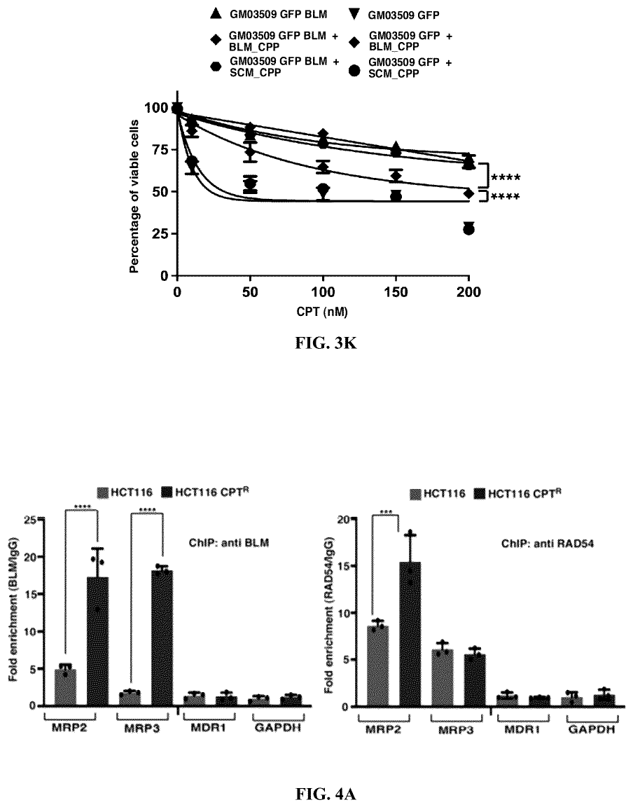

Next, it was determined whether the interaction of BLM with RAD54 altered the repair response to the DNA damage and thereby influenced cell growth. To study the effect of BLM-RAD54 interaction within the cells, a TAMRA-tagged cell-permeable peptide for BLM (181-212) (termed BLM_CPP) and the scrambled sequence (termed SCM_CPP) were generated. Both the peptides were linked to an N-terminal SV40-derived Nuclear Localization Signal (NLS). The peptides were tested in GM03509 GFP cells lacking BLM expression. Specifically, asynchronously growing GM03509 GFP cells were treated with either 180 nM BLM_CPP or SCM_CPP for 2 hours after which the peptides were washed off. The intake of the TAMRA tagged peptides was monitored by live cell imaging. Representative images show that both TAMRA tagged BLM_CPP and TAMRA tagged SCM_CPP entered the nucleus of GM03509 GFP cells. The cellular uptake of BLM (181-212) cell permeable peptide (BLM_CPP) and scrambled cell permeable peptide (SCM_CPP) is illustrated in FIG. 3A.

Next, GM03509 GFP cells were either grown in presence of HU (for 16 hours) or for 6 hours more after washing away HU, but in presence of either 180 nM BLM_CPP or SCM_CPP (FIG. 3B) or BLM_NP or SCM_NP (FIG. 3C). Lysates made were probed with antibodies against RAD54, RAD51, gH2AX, hsp90. The experiment was repeated three times and representative blots presented. The presence of both BLM_CPP and the nanoparticle coated BLM peptide (BLM_NP) led to the persistence of the endogenous levels of pro-recombination proteins, RAD51 and RAD54 even after the damage inducer hydroxyurea (HU) had been washed off (FIG. 3B, 3C). Consequently, the number of RAD51, RAD54 foci increased after treatment with BLM_CPP exposure as shown in (FIG. 3D). For the experiment in FIG. 3D, HCT116 BLM−/− cells were either grown in presence of HU (for 16 hours) or for 6 hours more after washing away HU, but in presence of either 180 nM BLM_CPP or SCM_CPP. The cells were fixed and processed for immunofluorescence with RAD54, RAD51 antibodies. The experiment was repeated three times and representative images presented in upper panel of FIG. 3D. The bottom panel of FIG. 3D shows quantitation: foci/cell. Mean±S.D. Number of cells analyzed=45.

To determine the biological consequences of the presence of BLM_CPP within the cells, GM03509 GFP-BLM and GM03509 GFP cells were treated with HU (16 hours) which arrested the cells in the G1/S boundary. Subsequently, HU was washed off and the cells were released for different time intervals in the presence of either 180 nM of BLM_CPP or SCM_CPP. Flow cytometry analysis revealed that BLM CPP allowed the cells to enter into proliferation mode much earlier than SCM_CPP as illustrated in FIG. 3E.

Next, GM03509 GFP-BLM and GM03509 GFP cells were treated with HU treatment (16 hours). Cells released post HU treatment were grown for 6 hours with (FIG. 3F) 180 nM of BLM_CPP or SCM_CPP or (FIG. 3G) BLM_NP or SCM_NP. Lysates made were probed with antibodies against p21, p27, hsp90. The experiment was repeated three times and one representative experiment has been shown. Western analysis revealed that compared to SCM_CPP or SCM_NP, the levels of cyclin-dependent kinase inhibitors, p21 and p27 were reduced when cells were treated with either BLM_CPP or BLM_NP (FIG. 3F, 3G), thereby indicating increased proliferation. This was accompanied by decreased levels of residual DNA damage as measured by γH2AX foci and protein levels (FIG. 3B, 3C, 3H) and Comet assays (FIG. 3I)

For the experiment in FIG. 3H, HCT116 BLM−/− cells were treated with HU treatment (16 hours). Cells released post HU treatment were grown for 4 hours with 180 nM of BLM_CPP or SCM_CPP, after which cells were fixed and processed for immunofluorescence with γH2AX antibody. The experiment was repeated three times and representative images presented (left panel). Quantitation of the number of foci/cell is indicated as Mean±S.D (right). Number of cells analyzed=45.

For the experiment in FIG. 3I, GM03509 GFP-BLM and GM03509 GFP cells were treated with HU treatment (16 hours). Cells released post HU treatment were grown for 6 hours with 180 nM of BLM_CPP or SCM_CPP, following which Comet assays were carried out. The data was from three independent experiments. Mean±S.D. The data shows that BLM_CPP decreased the levels of the cellular DNA damage.

To test whether the pro-proliferative effect might promote tumor resistance to chemotherapeutic drugs, isogenic lines GM03509 GFPBLM and GM03509 GFP cells were treated with 180 nM of BLM_CPP or SCM_CPP in presence of (FIG. 3J) 1 nM, 2 nM, 3 nM, 4 nM, 5 nM of CDDP or (FIG. 3K) 10 nM, 50 nM, 100 nM, 150 nM, 200 nM of CPT. The percentage of viable cells were determined by MTT assays. The data is from four independent experiments (FIG. 3J) or three independent experiments (FIG. 3K). Mean±S.D. Compared to GM03509 GFP-BLM, GM03509 GFP cells were more sensitive to both the tested drugs. However, GM03509 GFP cells pre-treated for 6 hrs with BLM_CPP (but not SCM_CPP) displayed resistance to the drugs (FIG. 3J, 3K).

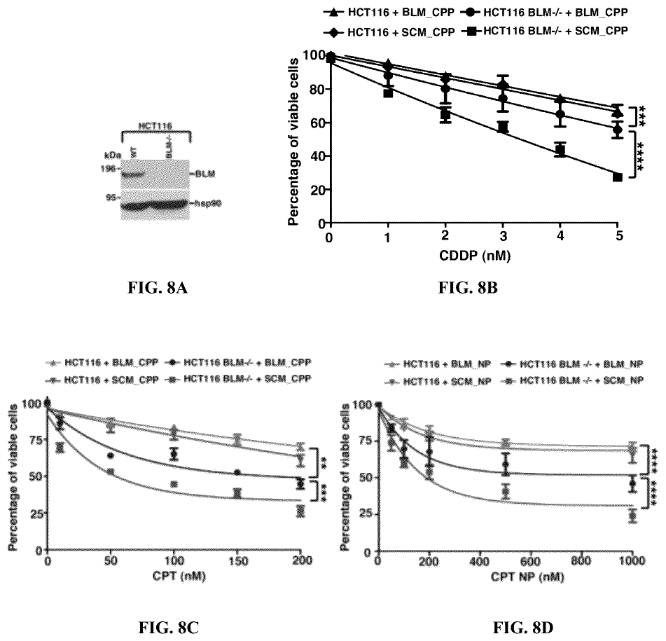

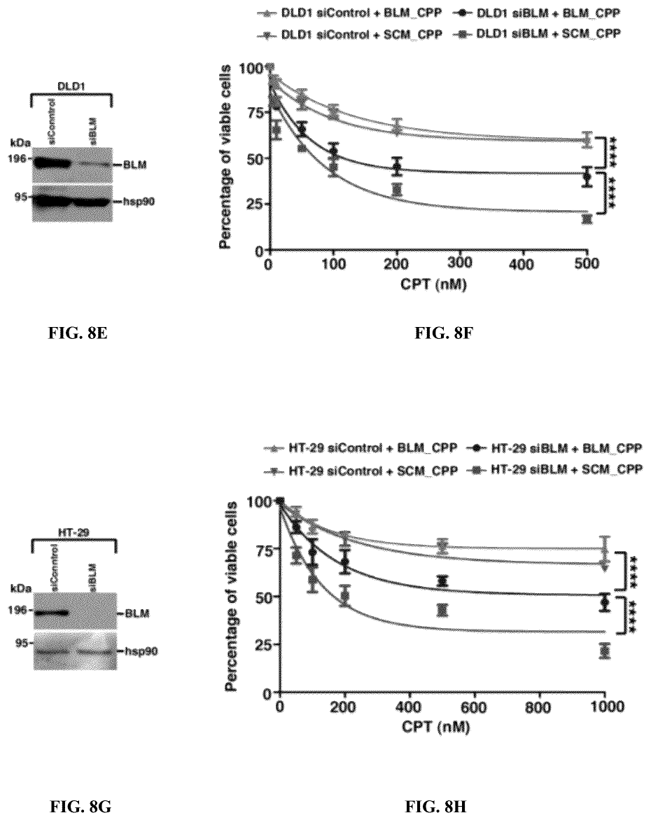

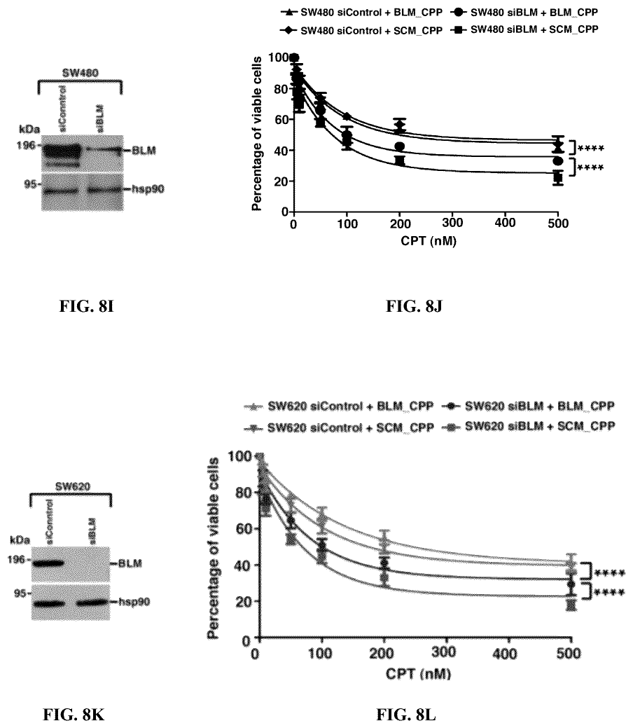

These findings were confirmed using five different colon cancer cells (HCT116, DLD1, HT-29, SW480, SW620) and their isogenic control in which the expression of BLM was ablated, for example, loss or depletion of BLM in colon cancer cells as shown in FIG. 8A, 8E, 8G, 8I, 8K. Specifically, lysates were made from (FIG. 8A) HCT116 WT, HCT116 BLM−/−, (FIG. 8E) DLD1 treated with either siControl or siBLM, (FIG. 8G) HT-29 treated with either siControl or siBLM, (FIG. 8I) SW480 treated with either siControl or siBLM and (FIG. 8K) SW620 treated with either siControl or siBLM, Western analysis was carried out with antibodies against BLM, hsp90. All the experiments were repeated three times and one representative experiment has been shown in each case. In all the cases, the addition of BLM_CPP or BLM_NP increased the resistance to CDDP, CPT or CPT_NP (FIG. 8B-8D, 8F, 8H, 8J, 8L). Specifically, HCT116 WT and HCT116 BLM−/− cells were treated with (FIG. 8B, 8C) 180 nM of BLM_CPP or SCM_CPP or (FIG. 8D) BLM_NP or SCM_NP in presence of (FIG. 8B) 1 nM, 2 nM, 3 nM, 4 nM, 5 nM of CDDP, (FIG. 8C) 10 nM, 50 nM, 100 nM, 150 nM, 200 nM of CPT, (FIG. 8D) 50 nM, 100 nM, 200 nM, 500 nM, 1000 nM of CPT. The percentage of viable cells were determined by MTT assays. The data is from three independent experiments. Mean±S.D.

### Example 4: BLM Enhanced RAD54 Mediated Chromatin Remodeling on MRP2 Gene Promoter

To determine whether the BLM/RAD54 interaction remodeled chromatin at the promoters of Multi Drug Resistance (MDR) transporters and thereby affected chemoresistance, GM03509 BLM Clone 9.6 cells were generated by correcting the mutation in BLM gene (c.1784C>A) in the GM03509 fibroblasts obtained from a BS patient. Homology Directed Repair (HDR) dependent CRISPR/Cas9 system was used for this purpose. Specifically, a PCR product containing the genomic DNA from GM03509 BLM Clone 9.6 cells spanning the mutated site was cloned into TA vector. Sanger sequencing was carried out in 20 sub-clones.

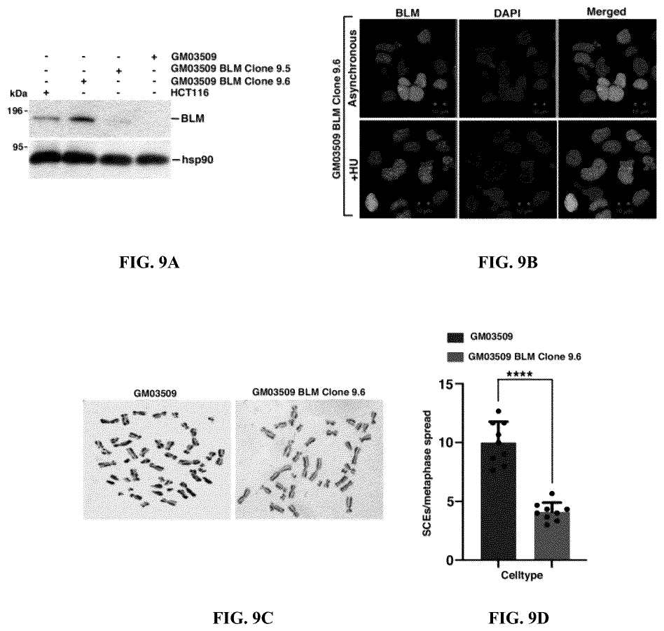

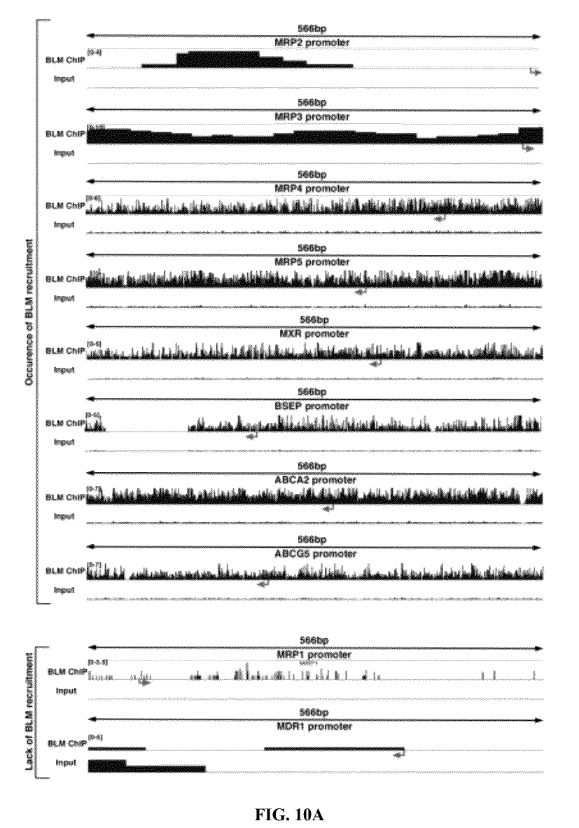

FIG. 9A shows that the expression of BLM protein in GM03509 BLM Clone 9.6 cells was similar to that observed in HCT116 WT cells. For this experiment, lysates were made from GM03509 BLM Clone 9.6 cells (GM03509 cells where BLM mutation has been corrected), GM03509 BLM Clone 9.5 cells (GM03509 cells where BLM mutation was not corrected completely, as a very low level of BLM expression is observed), GM03509 (parental BS fibroblast) and HCT116 WT cells. Western blot analysis was carried out using antibodies against BLM and hsp90 (FIG. 9A). The experiment was repeated three times and one replicate is shown in FIG. 9A.

FIG. 9B shows that the BLM protein in the CRISPR/Cas9 corrected cells also formed foci upon HU treatment. GM03509 BLM Clone 9.6 cells were treated with 1 mM of HU for 16 hours. Cells were fixed and processed for immunofluorescence with anti-BLM antibody. The experiment was repeated three times and a representative image is shown in FIG. 9B which shows that GM03509 BLM Clone 9.6 cells form BLM foci post HU treatment.

FIG. 9C and FIG. 9D show that the correction of BLM mutation in GM03509 BLM Clone 9.6 cells decreases the high levels of Sister Chromatin Exchanges (SCEs) seen in GM03509 cells. SCEs were carried out in GM03509 and GM03509 BLM Clone 9.6 cells. FIG. 9C shows representative images. FIG. 9D shows the quantitation of the data in FIG. 9C. The data is from three independent experiments (n=9 in each experiment). Mean±S.D.

Using GM03509 BLM Clone 9.6 cells, BLM ChIP-seq was performed to determine the regions where BLM is recruited in absence of any damage. The Circos plot obtained from BLM Chip-seq analysis carried out on GM03509 BLM Clone 9.6 cells showed the enrichment of BLM on various chromosomal locations.

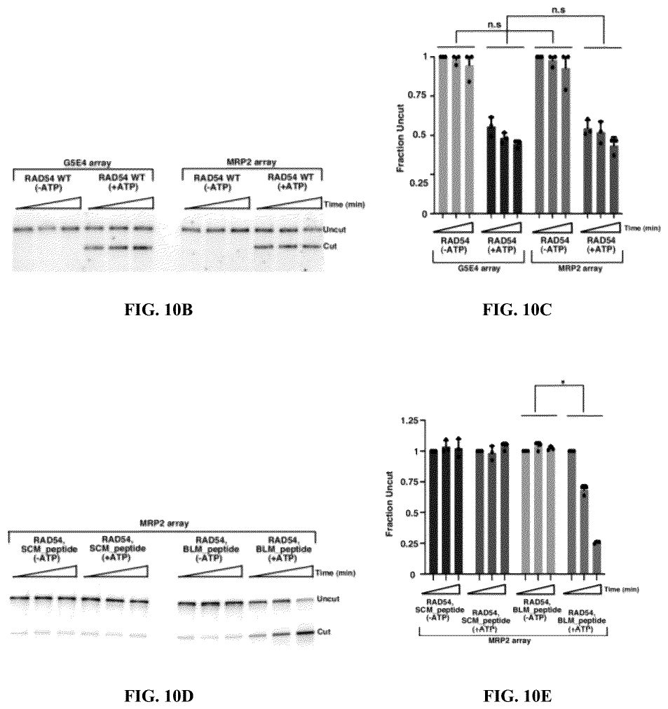

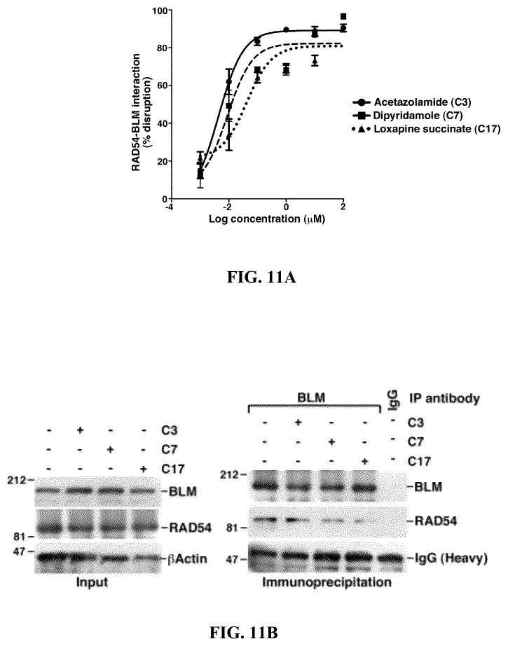

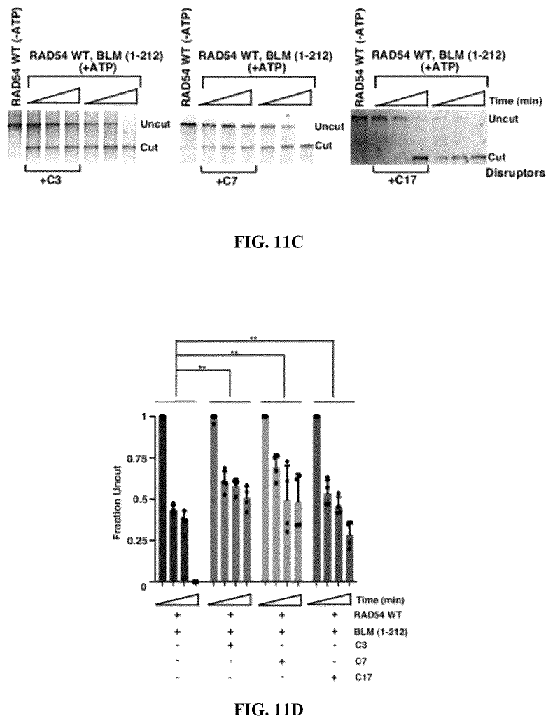

Next, it was determined whether BLM is recruited to different promoters within 5 kb of Transcription Start Site (TSS) of gene promoters. FIG. 10A shows the Integrative Genomics Viewer (IGV) browser tracking BLM peaks and input signals within 566 bp from TSS of MDR gene promoters where BLM is recruited: MRP2, MRP3, MRP4, MRP5, MXR, BSEP, ABCA2 and ABCG5 (upper panel) and MDR gene promoters where BLM is not recruited: MRP1, MDR1 (lower panel). The interval range is indicated in brackets on the left side of each track. Amongst others, enrichment could be specifically seen on various MDR gene promoters. In fact, BLM was recruited to eight of the ten tested MDR gene promoters, namely MRP2, MRP3, MRP4, MRP5, MXR, BSEP, ABCA2 and ABCG5.

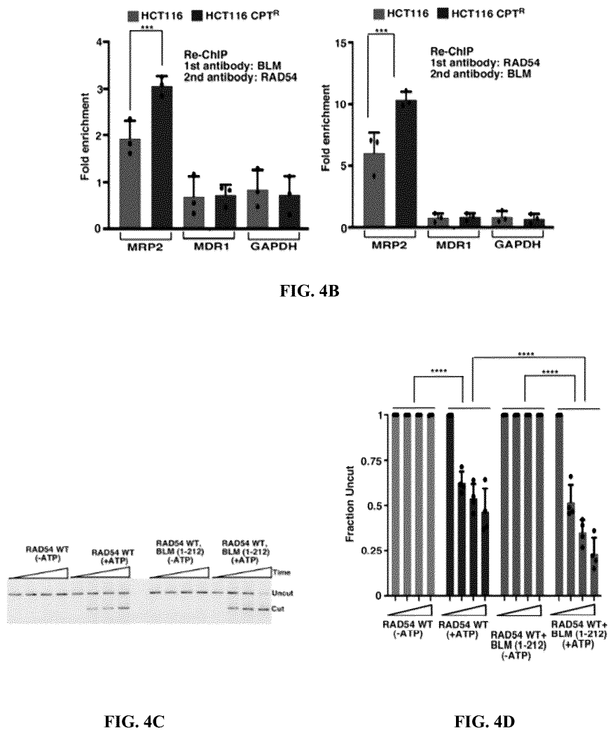

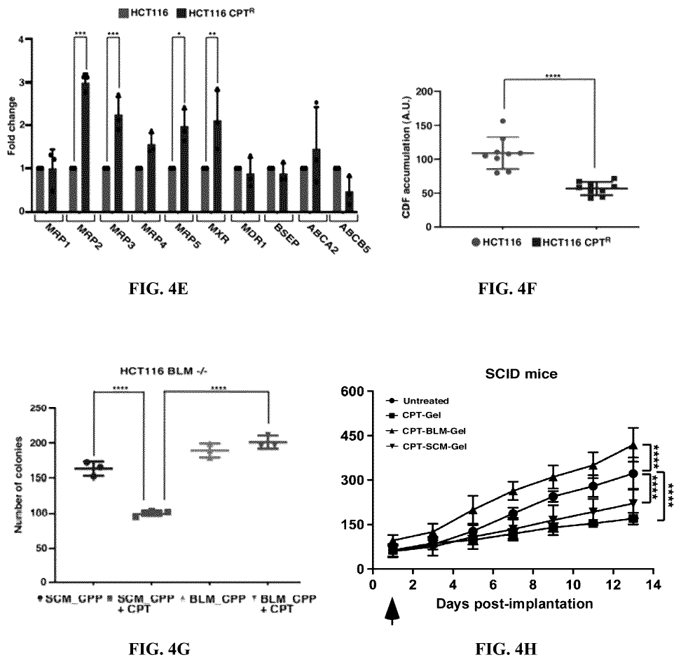

To determine the role of the MDR genes with respect to BLM/RAD54 interaction, a cell line was generated from HCT116 WT cells which was resistant to camptothecin (HCT116 IC60 CPTR). To determine whether the BLM-RAD54 complex was specifically recruited onto the MDR gene promoters, ChIP-qPCR experiments were performed by using BLM and RAD54 antibodies using the parental and resistant cells. The region used to check BLM/RAD54 recruitment by ChIP-qPCR was selected from the BLM ChIP-seq dataset. Both BLM and RAD54 were highly enriched only on MRP2 promoter. For this experiment, chromatin isolated from HCT116 WT and HCT116 IC60 CPTR cells was used for ChIP with either (left panel) anti-BLM or (right panel) anti-RAD54 antibody (FIG. 4A). The DNA obtained after ChIP was used to determine the enrichment on MRP2, MRP3, MDR1 and GAPDH promoters by qPCR. Re-ChIP experiments (FIG. 4B) further confirmed that BLM and RAD54 were both co-recruited onto the MRP2 promoter with higher occupancy seen in the resistant cells as compared to the wild type cells. For this, chromatin isolated from HCT116 WT and HCT116 IC60 CPTR cells was used for Re-ChIP with (left panel) first antibody RAD54 and second antibody BLM, (right panel) first antibody BLM and second antibody RAD54. The DNA obtained after Re-ChIP was used to determine the enrichment on MRP2, MDR1 and GAPDH promoters by qPCR. The data for both (FIG. 4A, 4B) are from three independent experiments. Mean±S.D.

An array for REA using sequences from the MRP2 promoter was created. It was observed that, in parallel assay conditions, the RAD54-mediated chromatin remodeling was equivalent in both G5E4 and MRP2 array. REA assays were carried out with chromatinized G5E4 or MRP2 array using the following experimental conditions: RAD54-ATP, RAD54+ATP. The reactions were stopped after 1 minute, 5 minutes and 10 minutes (FIG. 10B). FIG. 10C is the Quantitation of FIG. 10B. The fraction uncut is presented. The data was from three independent experiments. Mean±S.D. (FIG. 10B, 10C). More importantly, presence of BLM (1-212) significantly increased RAD54 mediated chromatin remodeling of the chromatinized MRP2 array (FIG. 4C, 4D). For this, REA assays were carried out with chromatinized MRP2 array using the experimental conditions as indicated. The reactions were stopped after 1 minute, 5 minutes and 10 minutes. FIG. 4D is the quantitation of FIG. 4C. The fraction uncut is presented. The data is from four independent experiments. Mean±S.D (FIG. 4C, 4D).

BLM_peptide (but not SCM_peptide) also enhanced RAD54 mediated remodeling activity on MRP2 substrate (FIG. 10D, 10E). Specifically, REA assays were carried out with chromatinized MRP2 array using the following experimental conditions: RAD54+SCM-peptide (−ATP), RAD54+SCM-peptide (+ATP), RAD54+BLM-peptide (−ATP), RAD54+BLM-peptide (+ATP). The reactions were stopped after 1 minute, 5 minutes and 10 minutes. FIG. 10E is the quantitation of FIG. 10D. The fraction uncut is presented. The data was from three independent experiments. Mean±S.D (FIG. 10D, 10E).

The enhanced remodeling by BLM/RAD54 complex, resulted in increased transcription of multiple MDR genes (including MRP2) in HCT116 IC60 CPTR cells (FIG. 4E). Specifically, RNA was isolated from HCT116 WT and HCT116 IC60 CPTR cells and used for RT-qPCR. The levels of MRP1, MRP2, MRP3, MRP4, MRP5, MXR, MDR1, BSEP, ABCA2, ABCB5 were quantitated from three independent experiments. Mean±S.D. In fact, the resistant cells also displayed higher MRP2 activity as compared to the HCT116 WT cells as seen by the lower levels of CDF florescence (FIG. 4F). Particularly, HCT116 WT IC60 CPTR cells have enhanced MRP2 efflux activity. Asynchronously growing HCT116 WT and HCT116 WT IC60 CPTR cells were incubated with MRP2 substrate (CDFD) for 30 minutes at 37° C. The accumulation of florescent product CDF was determined as a measure of MRP2 activity. The experiment was carried out nine times. Mean±S.D.

### Example 5: Interaction of BLM with RAD54 Enhanced Neoplastic Transformation

To determine whether enhanced chemoresistance due to RAD54-BLM had any effect on neoplastic transformation, it was first tested whether the presence of BLM_CPP had an effect on the anchorage independent growth of HCT116 BLM−/− cells. Indeed BLM_CPP enhanced the number of soft agar colonies even in the presence of CPT. Soft agar assay was carried out in HCT116 BLM−/− cells by treating them 180 nM of BLM_CPP or SCM_CPP in absence or presence of CPT (120 nM). The number of soft agar colonies in each condition were counted. The data is from three independent experiments. Mean±S.D. (FIG. 4G). Further, the proliferative capacity of BLM (181-212) was tested in SCID mice where tumors were developed by subcutaneously implanting HCT116 BLM−/− cells. Specifically, HCT116 BLM−/− cells were injected into SCID mice (n=7). The day when the approximate volume of the tumor was 50 mm3 was considered as Day 1 (indicated by arrow). On Day 1, the mice bearing 50 mm3 tumors were randomized into four groups: left untreated or injected at the base of the tumors with CPT entrapped in gel (CPT-Gel) or CPT and BLM peptide entrapped gel (CPT-BLM-Gel) or CPT and scrambled (SCM) peptide entrapped gel (CPT-SCM-Gel). Injection of CPT-Gel slowed down the tumor growth. However, the presence of CPT-BLM-Gel (but not CPT-SCM-Gel) enhanced the volume of the tumors (FIG. 4H). These results were further validated in another SCID mice-based xenograft model where tumor formation was monitored by implanting HCT116 BLM−/− stably expressing either GFP-BLM (181-212) or GFP alone. The day when the approximate volume of the tumor was 50 mm³ was considered as Day 1 (indicated by arrow). The volume of the tumors was estimated for the indicated days. Mean±S.D. Expression of GFP-BLM (181-212) augmented the rate of tumorigenesis (FIG. 4I) indicating that the 32 amino acid stretch in BLM which interacted with RAD54 promoted tumor growth even in the presence of the chemotherapeutic drug, CPT.

### Example 6: FDA Approved Small Molecules Disrupt BLM-RAD54 Interaction

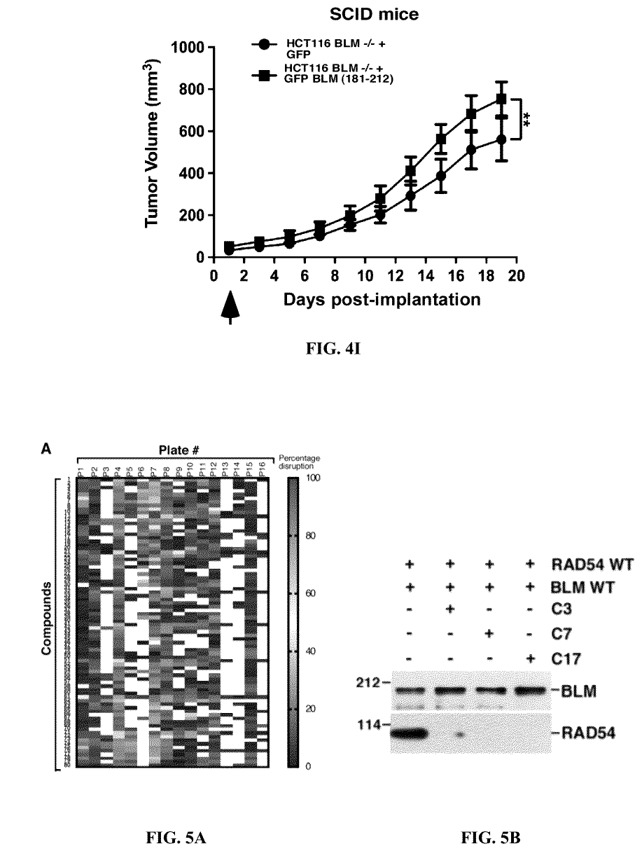

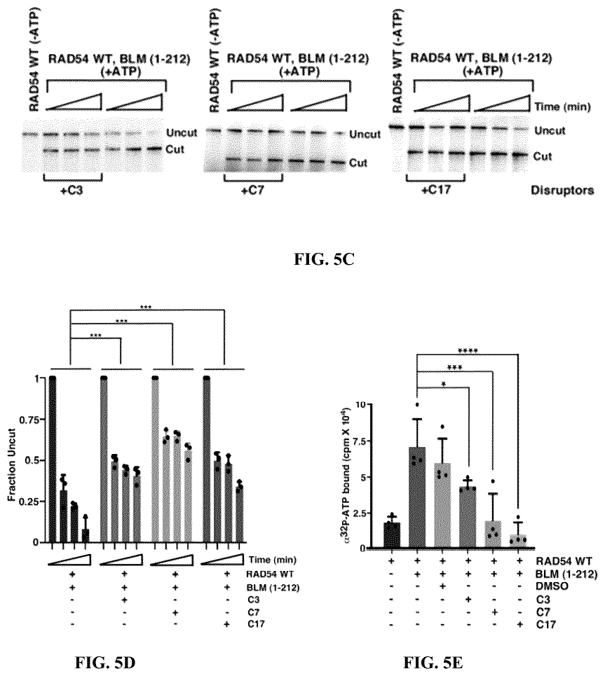

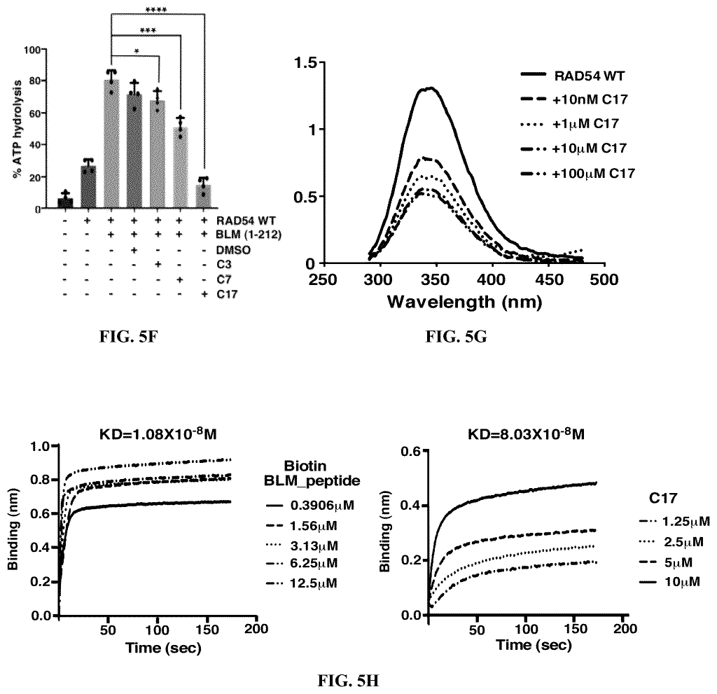

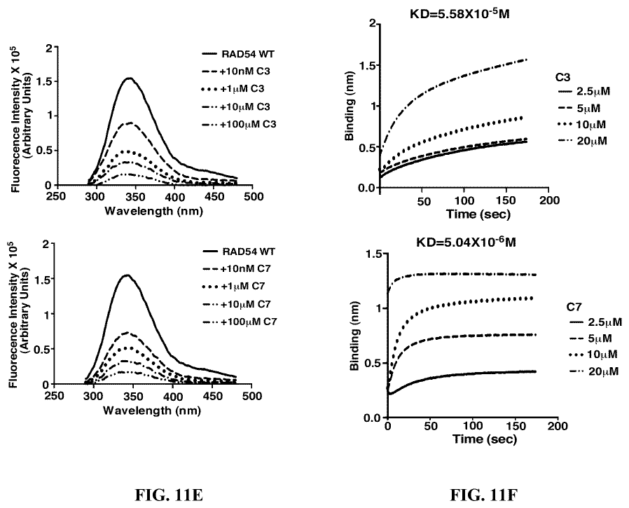

Having established that BLM-RAD54 interaction caused chemoresistance in colon cancer cells, it was determined whether breaking BLM-RAD54 interaction could re-sensitize colon cancer cells to the chemotherapeutic drugs. Using the Renilla luciferase-based protein complementation assay described earlier, 1280 FDA/EMA approved small molecules present in Prestwick chemical library were screened for their ability to disrupt BLM-RAD54 interaction. Each of the small molecules was incubated with HEK293T cells expressing BLM-F2 and N-RAD54-F1. Compared to cells expressing only BLM-F2 and N-RAD54-F1, the decrease in Renilla luciferase intensity in presence of each small molecule was monitored. Percentage disruption of the interaction between BLM F2 and N-RAD54 F1 was plotted in form of heatmap. The disruption of the BLM-RAD54 interaction was determined by a decrease in the Renilla luciferase activity as compared to control untreated cells. The extent of BLM-RAD54 disruption by all the tested compounds is shown in the form of heatmap (FIG. 5A). Seventeen compounds showed at least 70% disruption of RAD54-BLM interaction (at 10 μM concentration). Disruption (up to 12-20%) was observed even when the concentration of the disruptors was 1 nM (FIG. 11A, Table 1).

TABLE 1
Identity and information about the 17 compounds which disrupts BLM-RAD54
interaction in protein complementation assay

| Name of the | | | |
|---|---|---|---|
| compound | EC50 (nM) | CAS Number | Structure |
| Azaguanine-8 (C1) | 22.34 ± 1.975 | 134-58-7 | 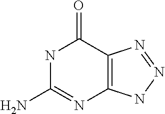 [SMILES: Nc1nc2[nH]nnc2c(=O)[nH]1] |
| Allantoin (C2) | 17.87 ± 1.817 | 97-59-6 | 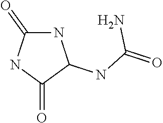 [SMILES: NC(=O)NC1NC(=O)NC1=O] |
| Acetazolamide (C3) | 4.204 ± 0.198 | 59-66-5 | 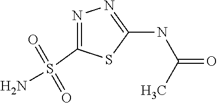 |
| Metformin hydrochloride (C4) | 3.013 ± 0.8124 | 1115-70-4 | 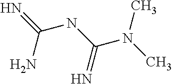 |
| Atracurum besylate (C5) | 5.376 ± 2.1727 | 64228-81-5 | 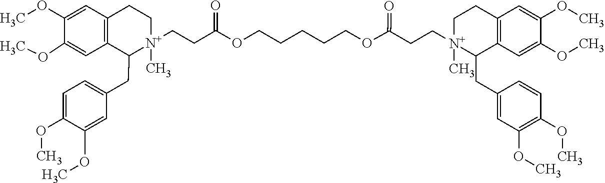 |
| Prednisone (C6) | 6.259 ± 1.999 | 53-03-2 | 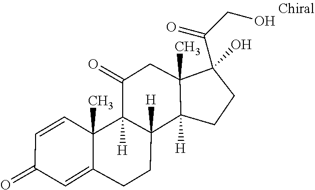 |
| Dipyridamole (C7) | 6.163 ± 0.297 | 58-32-2 | 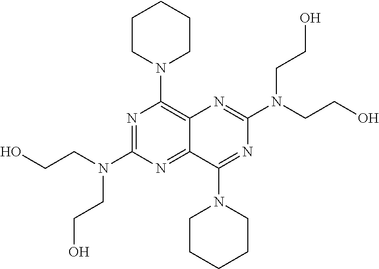 [SMILES: OCCN(CCO)c1nc(N2CCCCC2)c2nc(N(CCO)CCO)nc(N3CCCCC3)c2n1] |
| Metronidazole (C8) | 20.51 ± 2.606 | 443-48-1 | 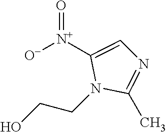 |
| Khellin (C9) | 16.66 ± 1.263 | 82-02-0 | 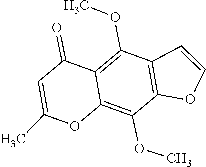 [SMILES: COc1c2occc2c(OC)c2c(=O)cc(C)oc12] |
| R(-) Apomorphine hydrochloride hemihydrate (C10) | 5.195 ± 1.335 | 41372-20-7 | 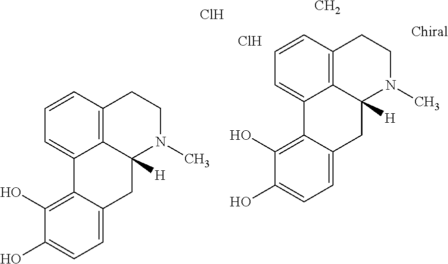 |
| Naloxone hydrochloride (C11) | 6.087 ± 2.249 | 357-08-4 | 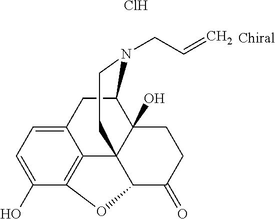 [SMILES: Cl ; C=CCN1CC[C@]23c4c5ccc(O)c4O[C@H]2C(=O)CC[C@@]3(O)[C@H]1C5] |
| Bromocryptine mesylate (C12) | 41.4 ± 4.459 | 22260-51-1 | 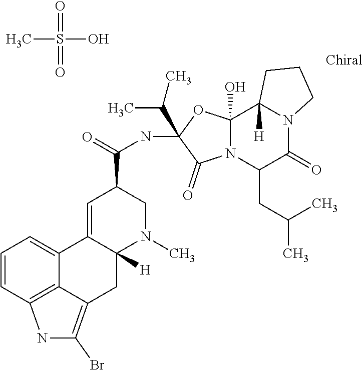 |
| Glipizide (C13) | 6.748 ± 3.688 | 29094-61-9 | 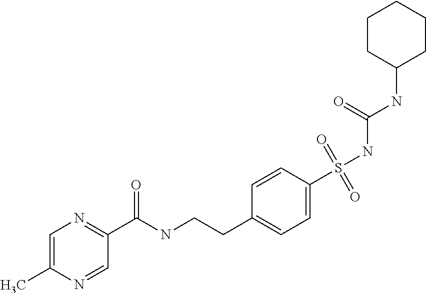 [SMILES: Cc1cnc(C(=O)NCCc2ccc(S(=O)(=O)NC(=O)NC3CCCCC3)cc2)cn1] |
| Verapamil hydrochloride (C14) | 4.093 ± 0.322 | 152-11-4 | 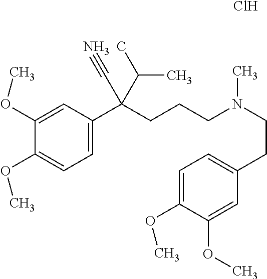 [SMILES: Cl ; COc1ccc(CCN(C)CCCC(C#N)(c2ccc(OC)c(OC)c2)C(C)C)cc1OC] |
| Erythromycin (C15) | 8.119 ± 3.888 | 114-07-8 | 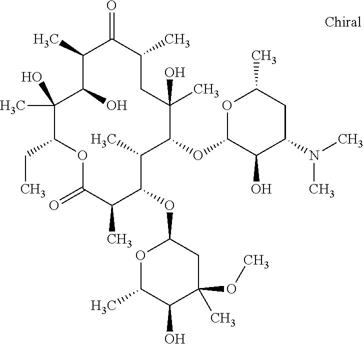 |
| Chloroxine (C16) | 26.41 ± 2.279 | 773-76-2 | 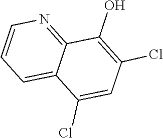 [SMILES: Oc1c(Cl)cc(Cl)c2cccnc12] |
| Loxapine succinate (C17) | 22.92 ± 4.247 | 27833-64-3 | 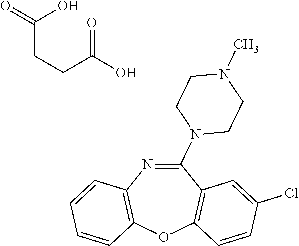 [SMILES: O=C(O)CCC(=O)O ; CN1CCN(C2=Nc3ccccc3Oc3ccc(Cl)cc32)CC1] |

For the data in FIG. 11A, Table 1, using PCA, dose response curve for C3, C7, C17 were determined using a range of concentrations (1 nM, 10 nM, 100 nM, 1 μM, 10 μM and 100 μM) of the three small molecules. The percentage disruption of the interaction between BLM F2 and N-RAD54 F1 was plotted as a non-linear regression line. The data was from three independent experiments. Mean±S.D. Of these, the three most potent compounds based on their EC50 values were Acetazolamide (C3), Dipyridamole (C7) and Loxapine Succinate (C17).

Both in vitro (FIG. 5B) and in cellulo (FIG. 11B) interaction assays confirmed that these three compounds disrupted the RAD54-BLM interaction. For the experiment in FIG. 5B, in vitro interactions were carried out between bound GST-BLM WT and soluble His-RAD54 WT in the absence or presence of 10 μM of C3 or C7 or C17. The levels of bound RAD54 were determined by immunoblotting with anti-RAD54 antibody (FIG. 5B). Next, compounds C3, C7, C17 were tested in cellulo (FIG. 11B). Lysates were made from HEK293T cells either left untreated or treated with 100 nM C3, C7, C17 for 72 hours. (Left) Direct westerns were carried out with antibodies against BLM, RAD54, bActin. (Right) immunoprecipitation (IP) was carried out with anti-BLM antibody. Westerns were carried out with anti-BLM, anti-RAD54 antibodies. The experiment was repeated three times and one representative experiment has been shown (FIG. 11B).

Furthermore, significant attenuation in the BLM-dependent enhancement of RAD54 mediated chromatin remodeling activity on G5E4 array was observed in presence of C3, C7 or C17 (FIG. 11C, 11D). Specifically, REA assays were carried out with chromatinized G5E4 array using the following experimental conditions: RAD54 WT+BLM (1-212), RAD54 WT+BLM (1-212)+C3, RAD54 WT+BLM (1-212)+C7, RAD54 WT+BLM (1-212)+C17. These reactions were carried out in presence of ATP. RAD54 WT (−ATP) was used as control. The reactions were stopped after 1 minute, 5 minutes and 10 minutes. FIG. 11D is the quantitation of FIG. 11C. The fraction uncut is presented. The data is from three independent experiments. Mean±S.D. The data shows that C3, C7, and C17 decrease the efficiency of BLM dependent enhancement of RAD54 chromatin remodeling activity.

A significant attenuation in the BLM-dependent enhancement of RAD54 mediated chromatin remodeling activity was also observed on MRP2 array in presence of C3, C7 or C17 (FIG. 5C, 5D). Specifically, REA assays were carried out as indicated. The reactions were stopped after 1 minute, 5 minutes and 10 minutes. FIG. 5D is the Quantitation of FIG. 5C. The fraction uncut is presented. The data is from three independent experiments. Mean±S.D. Compounds C3, C7, C17 decreased the efficiency of BLM dependent enhancement of RAD54 chromatin remodelling activity.

The presence of the disruptors also led to a decrease in the ATP binding to RAD54. Compounds C3, C7, C17 decreased BLM dependent enhancement of the binding of ATP by RAD54 (FIG. 5E) and thereby reduced the extent of ATP hydrolysis (FIG. 5F). Both effects probably contributing to the decrease in chromatin remodeling activity by these three small molecules (as seen in FIG. 5C, 5D, 11C, 11D).

The mechanism by which C3, C7, C17 affected BLM-RAD54 interaction was investigated. It was observed that C3, C7 and C17 quenched the tryptophan florescence of RAD54 even at 10 nM concentration (FIG. 5G, FIG. 11E). Specifically, tryptophan fluorescence assays were carried out with His-RAD54 WT, either alone or in presence of the indicated concentrations of C17 (FIG. 5G) or in presence of the indicated concentrations of C3, C7 (FIG. 11E). RAD54 fluorescence was measured in a fluorometer. The experiment was repeated three times and representative experiment has been shown in FIG. 5G and FIG. 11E. The results indicate that C3, C7, and C17 alter the conformation of RAD54. This suggested that these small molecules could disrupt BLM-RAD54 interaction by binding and altering the conformation of RAD54.

To quantitate the plausible interaction between C3, C7, C17 and RAD54 protein, binding kinetics was performed by using Bio-Layer Interferometry (BLI). Specifically, Octet BLI based studies were performed to determine the dissociation constant of the interaction of different concentrations of Biotin BLM_Peptide and C3, C7, or C17 with His-RAD54 WT immobilized onto Ni-NTA-sensor. The affinity constant (KD)±SD is shown. The experiment was repeated three times and one representative experiment has been shown in FIG. 5H and FIG. 11F. Increasing biotinylated BLM (181-212) peptide concentrations led to a sharp association curve with the bound His-RAD54. Moreover, this interaction quickly stabilized and plateaued for all the concentrations. This confirmed a stable interaction between RAD54 and BLM (181-212) with an affinity constant of 1.08×10⁻⁸M (FIG. 5H, left panel). Similar experiments were also performed to check the affinity for the compounds, C3, C7 and C17 towards bound His-RAD54. All three small molecules were able to bind RAD54 with varying affinities. C17 bound with the maximum affinity of 8.03×10⁻⁸M, which was comparable to that of the BLM peptide (KD=1.08×10⁻⁸ M) (FIG. 5H, right panel). C7 and C3 had affinity constants of 5.04×10⁻⁶M and 5.58×10⁻⁵M, respectively (FIG. 11F). The association curves for C17 represent an initial sharper binding followed by slow stabilization (FIG. 5H, right panel). In the case of C7, a complete saturation at its maximum concentration used was observed, while for C3 a gradual binding kinetics was observed (FIG. 11F). Hence, BLI analysis revealed the following order of affinity of the three drugs for RAD54: C17>C7>C3.

### Example 7: BLM-RAD54 Disruptors Reverted Chemoresistance

To understand the biological significance of this disruptions, the effect of C3, C7, C17 was examined on the viability of three resistant lines, namely HCT116 IC60 CPTR, HCT116 IC60 CDDPR (lines resistant to CPT and CDDP, created for this study) and HCT116 1-OHPR (Yang A D, Fan F, Camp E R, van Buren G, Liu W, Somcio R, et al. Chronic oxaliplatin resistance induces epithelial-to-mesenchymal transition in colorectal cancer cell lines. Clin Cancer Res. 2006; 12(14 Pt 1):4147-53.). The three resistant lines and their wildtype counterpart HCT116 were exposed to a gradient of CPT, CDDP or 1-OHP. Treatment with all molecules led to a reduction in the resistance of HCT116 IC60 CPTR, HCT116 IC60 CDDPR, HCT116 IC60 1-OHPR (FIGS. 11A-11C) with a corresponding decrease in the EC50 values (Tables 2 and 3).

TABLE 2
EC50 values of HCT116 WT and CPT and CDDP resistant HCT116
derivatives upon treatment with either C3, C7 or C17
A. Effect of C3 on HCT116 WT and HCT116 CPT^{R}

| | | HCT116 | HCT116 WT | HCT116 WT |
|---|---|---|---|---|
| | HCT116 WT | WT + C3 | CPT^{R} | CPT^{R} + C3 |
| EC50 (μM) | 42.99 ± 3.697 | 34.14 ± 4.112 | 354.3 ± 5.614 | 32.91 ± 2.794 |

| | | |
|---|---|---|
| p value | 0.3179 | 0.0005 |
| (t-test) | | |

B. Effect of C7 on HCT116 WT and HCT116 CPT^{R}

| | | HCT116 | HCT116 WT | HCT116 WT |
|---|---|---|---|---|
| | HCT116 WT | WT + C7 | CPT^{R} | CPT^{R} + C7 |
| EC50 (μM) | 42.99 ± 3.697 | 19.5 | 354.3 ± 5.614 | 38.762 |

| | | |
|---|---|---|
| p value | 0.0597 | 0.00018 |
| (t-test) | | |

C. Effect of C17 on HCT116 WT and HCT116 CPT^{R}

| | | HCT116 | HCT116 WT | HCT116 WT |
|---|---|---|---|---|
| | HCT116 WT | WT + C17 | CPT^{R} | CPT^{R} + C17 |
| EC50 (μM) | 42.99 ± 3.697 | 32.03 | 354.3 ± 5.614 | 47.41 |

| | | |
|---|---|---|
| p value | 0.06412 | 0.009342 |
| (t-test) | | |

D. Effect of C3 on HCT116 WT and HCT116 CDDP^{R}

| | | HCT116 | HCT116 WT | HCT116 WT |
|---|---|---|---|---|
| | HCT116 WT | WT + C3 | CDDP^{R} | CDDP^{R} + C3 |
| EC50 (μM) | 3.78 | 3.008 | 21.72 | 4.137 |

| | | |
|---|---|---|
| p value | 0.9854 | 0.03541 |
| (t-test) | | |

E. Effect of C7 on HCT116 WT and HCT116 CDDP^{R}

| | | HCT116 | HCT116 WT | HCT116 WT |
|---|---|---|---|---|
| | HCT116 WT | WT + C7 | CDDP^{R} | CDDP^{R} + C7 |
| EC50 (μM) | 3.78 | 1.662 | 21.72 | 0.7216 |

| | | |
|---|---|---|
| p value | 0.05323 | 0.00183 |

(t-test)

F. Effect of C17 on HCT116 WT and HCT116 CDDP^{R}

| | | HCT116 | HCT116 WT | HCT116 WT |
|---|---|---|---|---|
| | HCT116 WT | WT + C17 | CDDP^{R} | CDDP^{R} + C17 |
| EC50 (μM) | 3.78 | 2.81 | 21.72 | 3.78 |

| | | |
|---|---|---|
| p value | 0.0786 | 0.00439 |

(t-test)

TABLE 3
HCT116 WT and HCT116 1-OHP^{R} cells upon treatment with C3, C7 or C17

| | | | | | | HCT11 | HCT11 | HCT11 |
|---|---|---|---|---|---|---|---|---|
| | | HCT11 | HCT11 | HCT11 | HCT11 | 61 − | 61 − | 61 − |
| | HCT11 | 6 WT + | 6 WT + | 6 WT + | 61 − | OHP^{R} + | OHP^{R} + | OHP^{R} + |
| | 6 WT + | 1-OHP + | 1-OHP + | 1-OHP + | OHP^{R} + | 1−OHP + | 1−OHP + | 1−OHP + |
| | 1-OHP | C3 | C7 | C17 | 1−OHP | C3 | C7 | C17 |
| EC50 | 4.769 ± | 3.716 ± | 4.935 ± | 3.135 ± | 14.1 ± | 7.06 ± | 15.1 ± | 5.172 ± |
| (μM) | 0.0367 | 0.0775 | 0.03757 | 0.0337 | 1.402 | 2.66 | 1.593 | 1.526 |
| p-value | | 0.0586 | 0.0722 | 0.0714 | | 0.0247 | 0.0055 | 0.0251 |
| (t-test) | | | | | | | | |

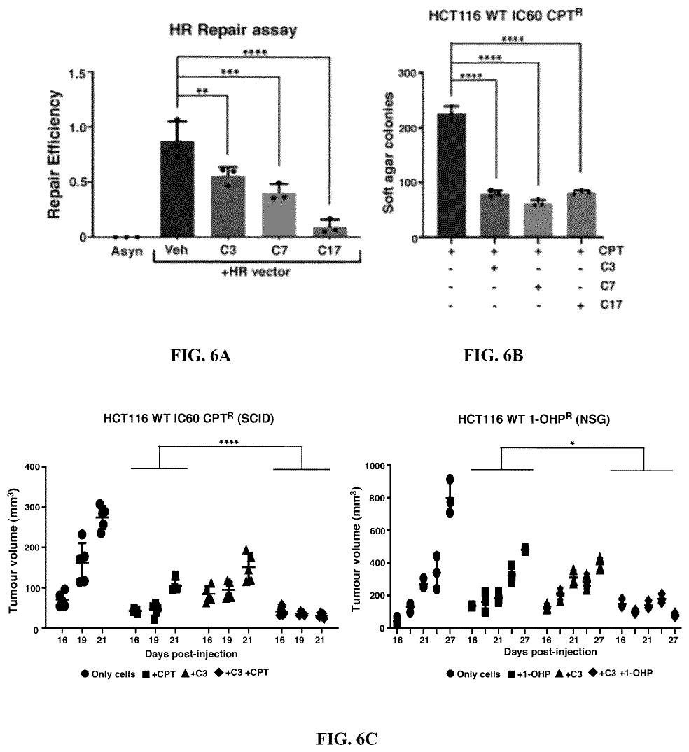

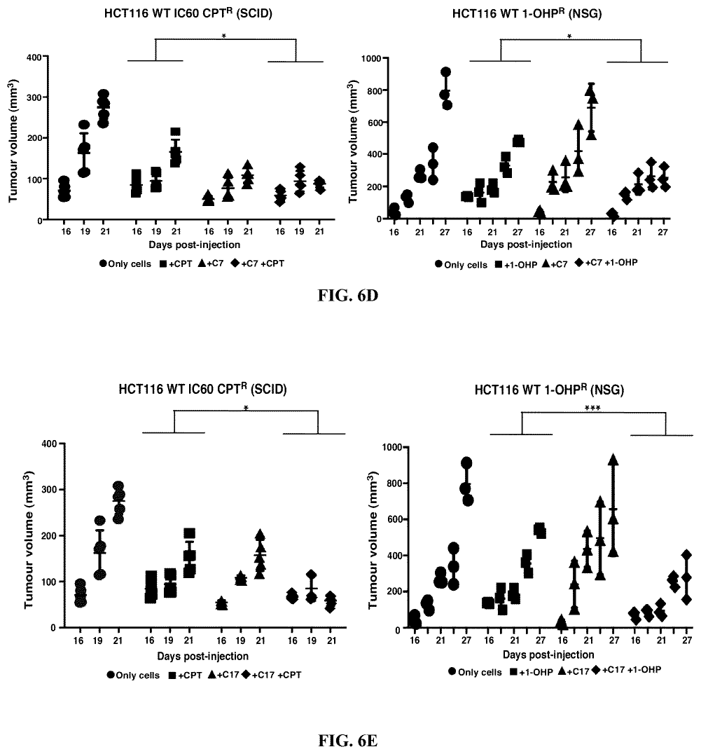

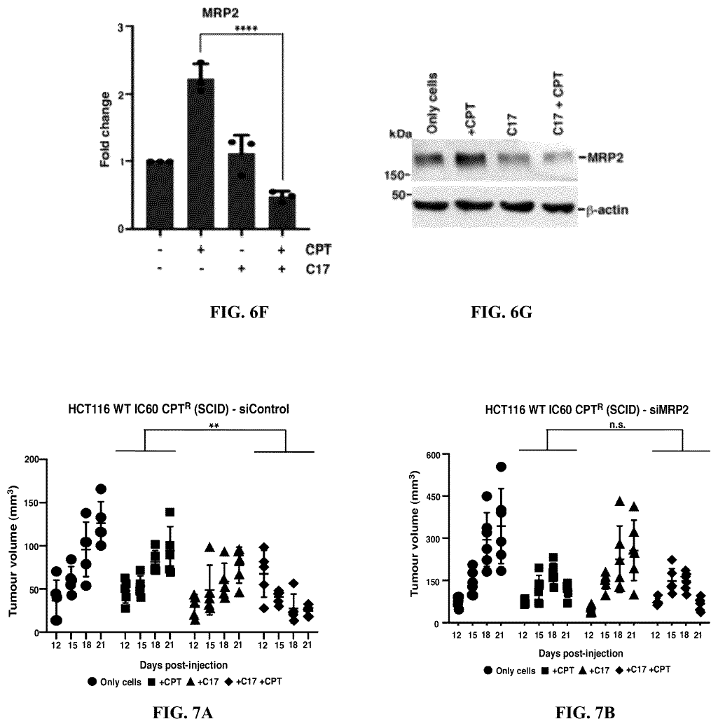

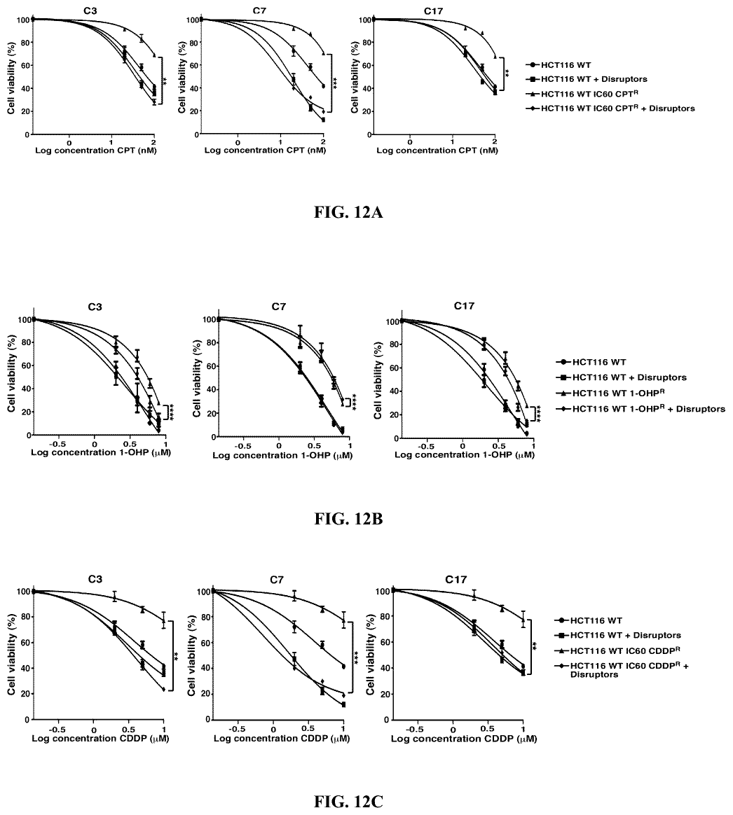

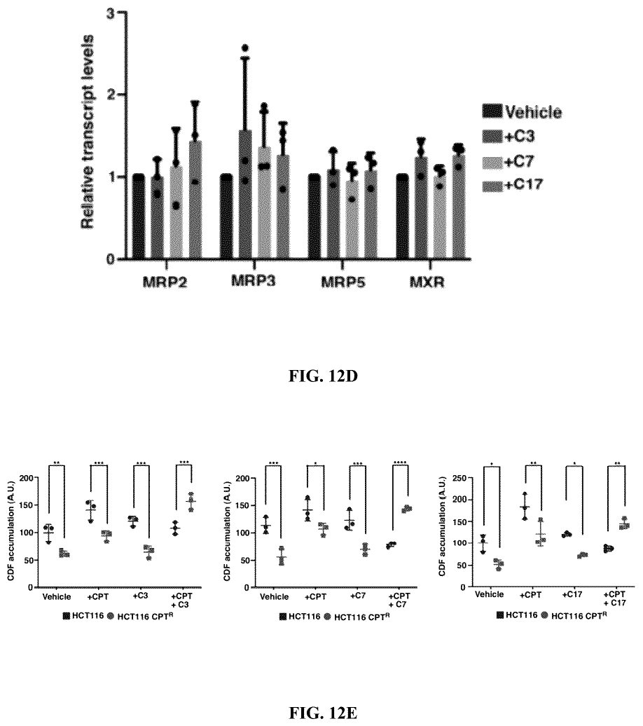

Specifically, HCT116 IC60 CPTR or HCT116 cells, HCT116 IC60 1-OHPR or HCT116 cells, HCT116 IC60 CDDPR or HCT116 cells were treated with 100 nM of C3, C7, C17 along with increasing concentrations of CPT or 1-OHP or CDDP for 72 hours. Concentrations of CPT used: 0.140 nM, 20 nM, 50 nM, 1000 nM. Concentration of 1-OHP: 0.14 μM, 2 μM, 4 μM, 6 μM, 8 μM. Concentrations of CDDP used: 0.140 μM, 2 μM, 5 μM, 10 μM. The percentage of viable cells were determined by MTT assays (FIG. 12A-12C). The data in FIG. 12A and FIG. 12C is from three independent experiments and the data in FIG. 12B is from nine independent experiments. Mean±S.D. However, these compounds themselves did not cause any statistically significant change in the expression levels of MDR genes. Particularly, small molecules do not alter the transcript levels of MDR genes in HCT116 IC60 CPTR cells (FIG. 12D). For the experiment in FIG. 12D, RNA isolated from HCT116 IC60 CPTR cells after 72 hours treatment with 100 nM of C3, C7 and C17 were used for RT-qPCR. The levels of MRP2, MRP3, MRP5, MXR were quantitated from three independent experiments. Mean±S.D. It was observed that the treatment with the three drugs led to a decrease in the rate of HR repair. Specifically, HCT116 IC60 CPTR cells were transfected with the HR substrate for 72 hours and the levels of HR was determined (FIG. 6A). The data is from three independent experiments. Mean±S.D. The results in FIG. 6A show that compounds C3, C7, C17 decreased the levels of the HR in HCT116 IC60 CPTR cells. Together with the effect on cell viability, these compounds diminish anchorage independent growth of camptothecin treated HCT116 IC60 CPTR cells (FIG. 6B). Specifically, soft agar assay was carried out in HCT116 IC60 CPTR cells by treating them with 100 nM of C3, C7, C17 along with 120 nM of CPT. The number of soft agar colonies in each condition were counted. The data is from three independent experiments. Mean±S.D. The data in FIG. 6B shows that compounds C3, C7, C17 decreased anchorage independent cell growth of HCT116 IC60 CPTR cells.

Further experiments were performed to understand whether the reverted chemoresistance due to C3, C7, and C17 was via modulation of MRP2 activity. As expected HCT116 IC60 CPTR cells displayed higher MRP2 activity as compared to HCT116 WT cells. Even upon treatment with only CPT or only small molecules—the same effect was observed. However, the combinatorial treatment of C3/C7/C17 with CPT led to enhanced accumulation of CDF dye suggesting a decreased MRP2 activity in presence of the three compounds. HCT116 WT and HCT116 WT IC60 CPTR cells were seeded and were either left untreated, or treated with CPT, or C3/C7/C17 alone or CPT+C3/C7/C17. The cells were incubated with MRP2 substrate CDFD for 30 minutes at 37° C. The accumulation of florescent product CDF was determined as a measure of MRP2 activity (FIG. 12E). The data is from three independent experiments. Mean±S.D.

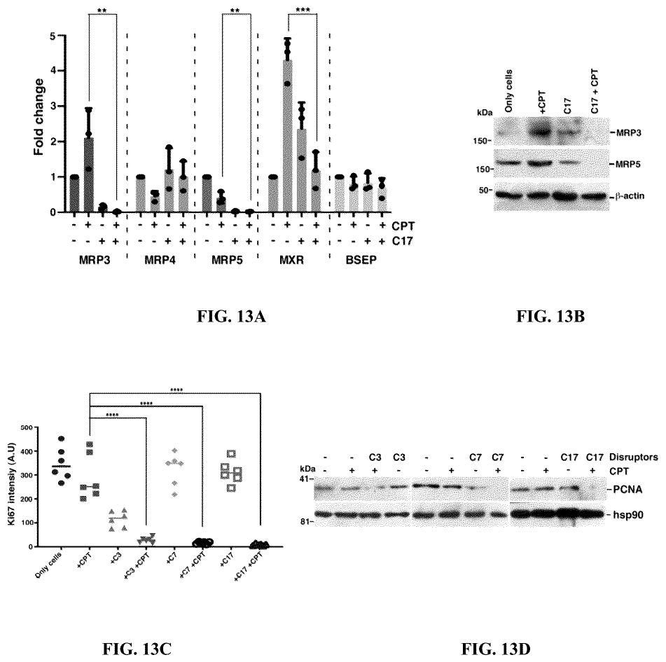

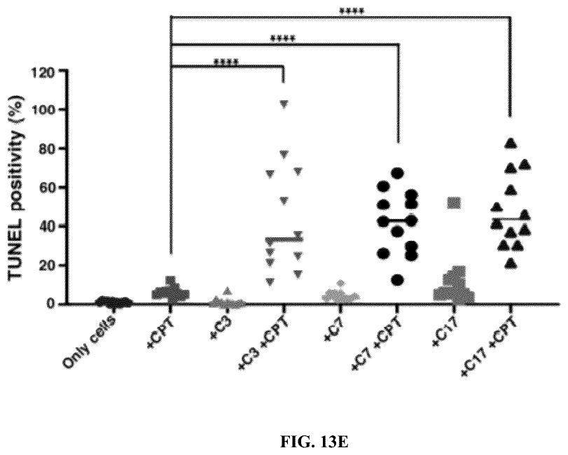

Finally, it was evaluated whether C3, C7, C17 reverted chemoresistance and thereby allowed better efficacy of CPT and 1-OHP in mouse xenograft models. Tumors were generated in either SCID or NSG mice using HCT116 IC60 CPTR or HCT116 IC60 1-OHPR cells implanted subcutaneously and injected with either CPT or 1-OHP alone or in combination with C3, C7, C17. As compared to the mice treated with CPT or 1-OHP alone, the dual treatment of either of the drugs with C3/C7/C17 inhibited the tumor growth of both HCT116 IC60 CPTR or HCT116 IC60 1-OHPR cells (FIG. 6C-6E). Tumors from different groups were excised at the end of the experiments. Both RNA and protein levels of multiple MDR genes (including MRP2) were very significantly decreased in tumors which have been co-treated with CPT and C17 (FIG. 6F, 6G, 13A, 13B). For these experiments, RNA and protein was isolated from tumors obtained at the end point of the xenograft experiment. RNA and the protein levels of MRP2 were determined by RT-qPCR and western blotting with anti-MRP2 antibody. For each group three tumor samples were analyzed (FIG. 6F, 6G). For the experiment in FIG. 13A, RNA was isolated from tumors obtained by injecting only HCT116 IC60 CPTR cells, tumors treated with CPT (1.25 mg/kg), tumors treated with C17 (5 mg/kg), and tumors treated with CPT and C17. The levels of the indicated MDR genes were quantitated from three tumor samples from each group. Mean±S.D. (FIG. 13A). The results indicate that the treatment with both CPT and C17 decreased the transcript levels of multiple MDR genes. For the experiment in FIG. 13C, lysates were made from tumors obtained by injecting only HCT116 WTIC60 CPTR cells, tumors treated with CPT (1.25 mg/kg), tumors treated with C17 (5 mg/kg), tumors treated with CPT and C17 and probed with antibodies against MRP2, MRP5 and β-actin. The experiment was repeated three times and one representative experiment has been shown (FIG. 13B). The results in FIG. 13B indicate that treatment with both CPT and C17 decreased MDR protein levels.

Decreased cell proliferation, as seen by decreased levels of Ki67 and PCNA levels (FIG. 13C, FIG. 13D) and increased apoptosis, as observed by the enhanced TUNEL positivity (FIG. 13E) were observed in tumors that had received the dual treatment (CPT/1-OHP and C3/C7/C17), thus demonstrating the importance of disruption of RAD54-BLM interaction in enhancing the therapeutic response to frontline chemotherapeutic drugs used for the treatment of colon cancer. For these experiments, tumors were obtained from the following groups: tumors made by injecting only HCT116 IC60 CPTR cells, tumors treated with CPT (1.25 mg/kg) alone, tumors treated with C3/C7/C17 (all 5 mg/kg), and tumors treated with CPT and C3/C7/C17. Sections from FFPE embedded tumors were either stained with anti-Ki67 antibody and the staining intensity determined by ImageJ software (n=6) (FIG. 13C) or subjected to TUNEL assay and the number of positive cells per area in μm2 depicting the TUNEL positive apoptotic cells determined (n=12) (FIG. 13E). Mean±S.D. Lysates made from these tumors were probed with antibodies against PCNA and hsp90 (FIG. 13D).

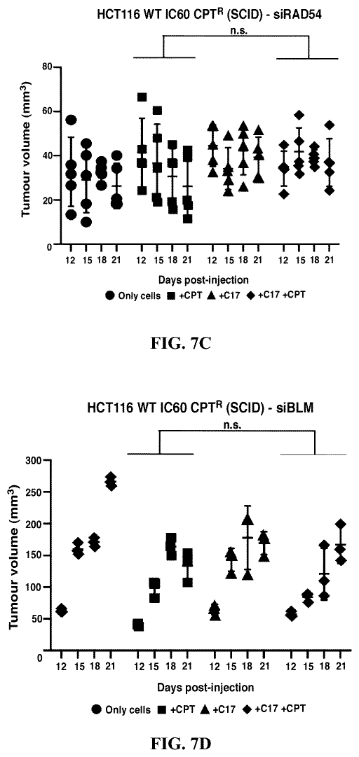

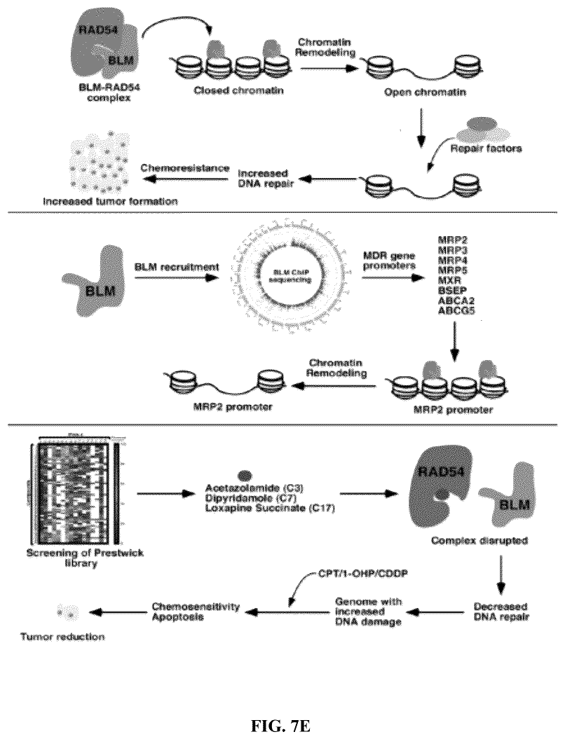

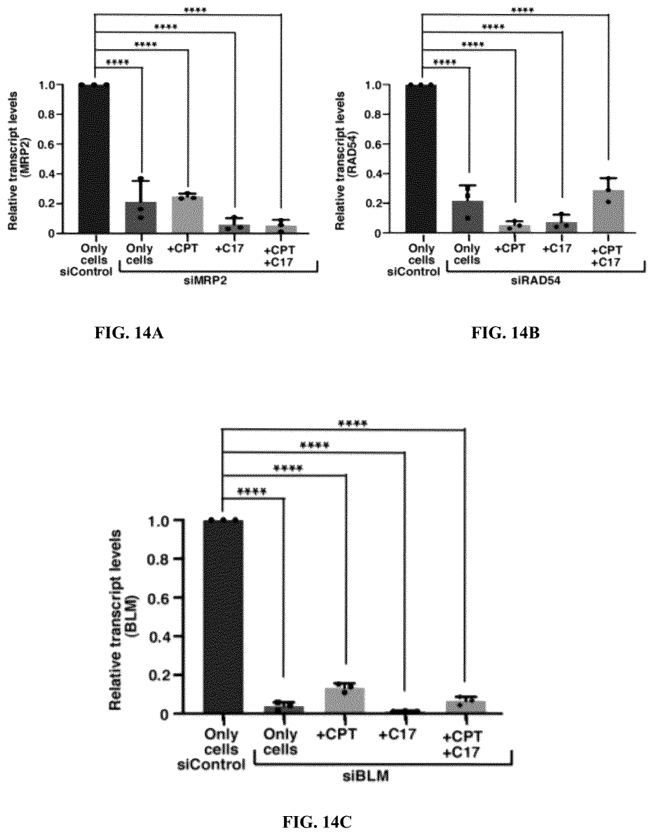

To determine whether the three key players (MRP2, BLM and RAD54) were indispensable for reverting chemoresistance in colon cancer, siRNA-based ablation in xenograft studies was carried out using SCID mice. Once the tumor volume reached 50 mm³, either siControl or siRAD54 or siBLM or siMRP2 were injected at the base of the tumor using TAC6 polymer mediated in vivo delivery. The experiment was stopped after 21 days after which the levels of RAD54, BLM, MRP2 transcripts were analyzed by RT-qPCR to validate the downregulation of the cognate genes (FIG. 14A-14C). As expected, compared to the use of only CPT, usage of both C17 and CPT in mice injected with siControl led to decreased tumor volume (FIG. 7A). Lack of MRP2 led to decreased tumor development upon C17 and CPT treatment. This is probably because of greater retention of CPT inside the tumors and thereby allowing increased chemotherapeutic potential of the drug (FIG. 7B). Loss of RAD54 did not lead to any tumor growth under any of the four conditions (FIG. 7C), probably because of the role of RAD54 during proliferation and maintenance of genome stability (Mills K D, Ferguson D O, Essers J, Eckersdorff M, Kanaar R, and Alt F W. Rad54 and DNA Ligase IV cooperate to maintain mammalian chromatid stability. Genes Dev. 2004; 18(11):1283-92.). Importantly, ablation of BLM rescued tumor growth even in presence of both C17 and CPT (FIG. 7D). This indicated that chemo-resistance to camptothecin was primarily mediated by BLM (and via its effect on RAD54).

### Example 8: Lack of Amino Acids (181-212) in BLM Abrogates the Functions of C3, C7, C17 in Causing Chemosensitivity to Camptothecin

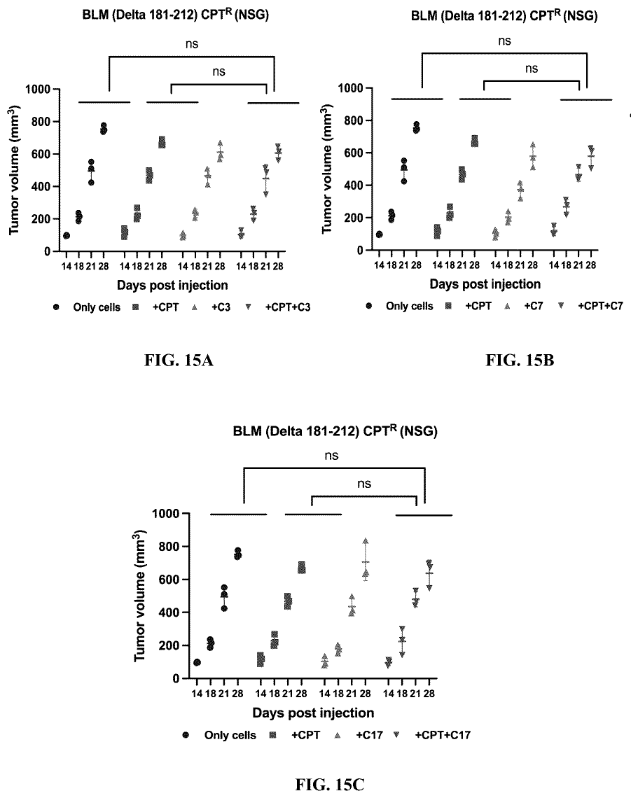

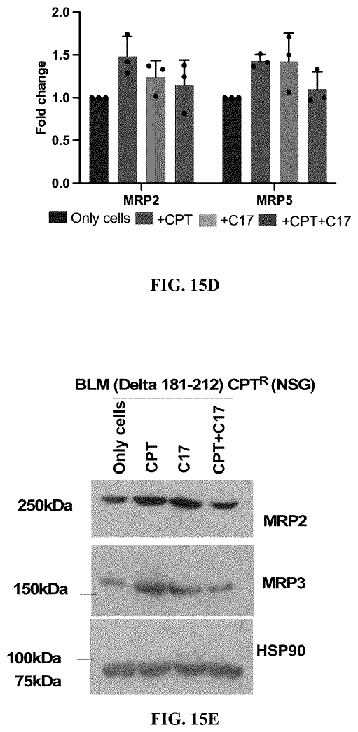

Xenografts assays were carried out in NSG mice using HCT116 CPT(R) cells stably expressing BLM (Delta 181-212) in BLM−/− background. FIGS. 15A-15C show the tumour volumes post-injection of cells. The mice were either left untreated, or treated with camptothecin (CPT) or C3/C7/C17 or CPT+C3/C7/C17. Lack of amino acids (181-212) in BLM abrogates the functions of C3, C7, C17 in causing chemosensitivity to camptothecin.

FIG. 15D and FIG. 15E shows that the levels of the MDR genes were unaffected in xenografts obtained from tumours from HCT116 CPT(R) cells expressing BLM (Delta 181-212) in BLM−/− background without or with C17 treatment. RNA (FIG. 15D) or protein (FIG. 15E) levels of the indicated MDR genes were determined by RT-qPCR or immunoblotting with the indicated antibodies.

### Example 9: Presence of C3, C7, and C17 Reverses Chemoresistance in a Xenograft Model Expressing HT-29 OHP(R) Cells

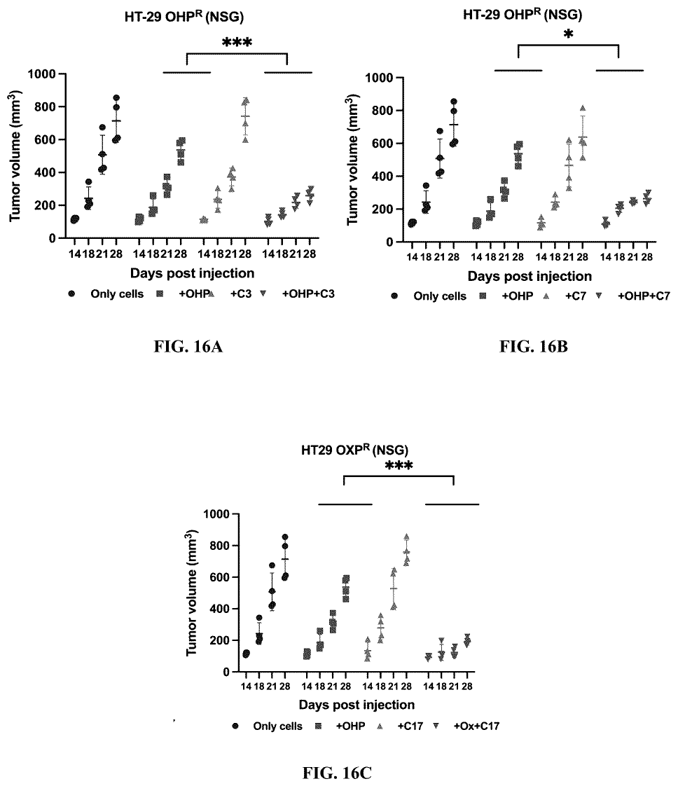

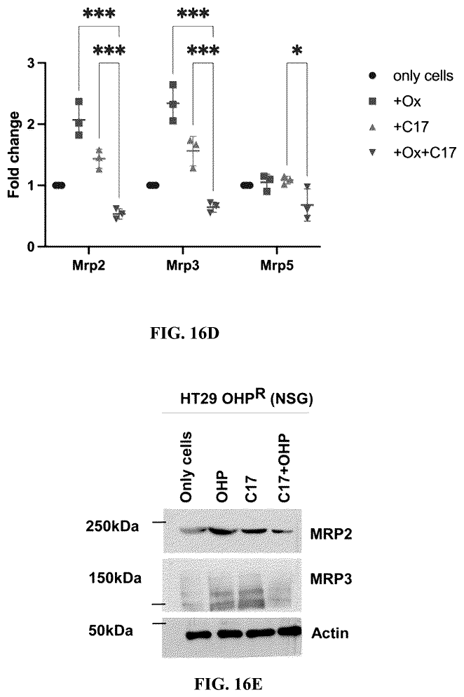

Xenografts assays were carried out in NSG mice using HT-29 OHP(R) cells. The mice were either left untreated, or treated with camptothecin (CPT) or C3/C7/C17 or CPT+C3/C7/C17. The tumour volumes post-injection of cells are shown in FIGS. 16A-16C. Treatment of C3, C7, C17 reverses chemoresistance in HT-29 OHP(R) cells derived xenograft model.

Levels of the MDR genes were decreased in xenografts obtained in tumours from HT-29 OHP(R) cells without or with C17 treatment. RNA (FIG. 16D) or protein (FIG. 16E) levels of the indicated MDR genes were determined by RT-qPCR or immunoblotting with the indicated antibodies.

### Example 10: Presence of C3, C7, and C17 Reverses Chemoresistance in a Syngeneic or Allogenic Model Expressing CT26 Cells

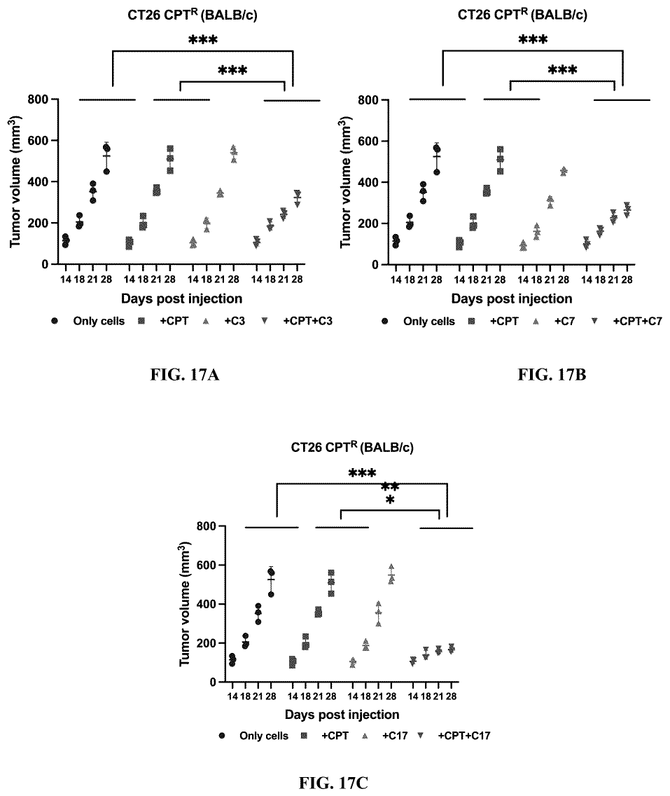

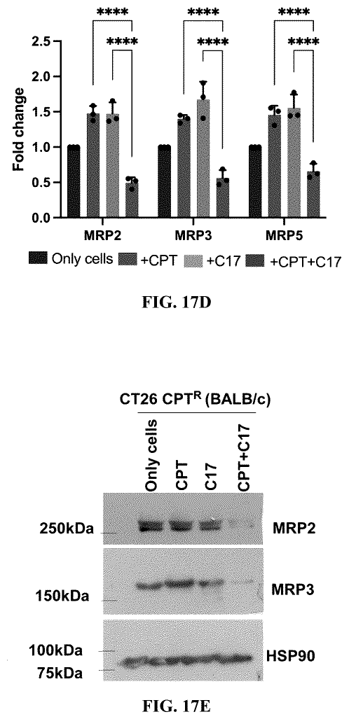

Xenografts assays were carried out in BALB/c mice using CT26 cells. The mice were either left untreated, or treated with camptothecin (CPT) or C3/C7/C17 or CPT+C3/C7/C17. The tumour volumes post-injection of cells are shown in FIG. 17A-17C. Treatment of C3, C7, C17 reverses chemoresistance in CT26 cells derived syngeneic or allogenic model.

Levels of the MDR genes were decreased in tumours obtained from a syngeneic or allogenic model using CT26 cells without or with C17 treatment. RNA (FIG. 17D) or protein (FIG. 17E) levels of the indicated MDR genes were determined by RT-qPCR or immunoblotting with the indicated antibodies.

### Experimental Methods

### Preparation of Primary Cell Culture

All HCT116 derived cells were grown in McCoy's 5A media, HT-29, DLD1, HEK293T, GM03509 GFP-BLM, GM03509 GFP were grown in DMEM while SW480, SW620 were grown in L15 medium. Cells were grown in 10% Fetal bovine serum supplemented with glutamine and anti-mycotic and anti-bacterial antibiotics. To generate stable lines EGFP-C1 or pEGFP-C1 NLS BLM (181-212) were transiently transfected in HCT116 BLM−/− cells using Lipofectamine 2000. Forty-eight hours post-transfection, cells were selected with G418 (1 mg/ml) for 5 days after which the mass obtained was maintained in presence of 200 μg/ml G418. All cells were tested to be free from mycoplasma contamination.

To generate camptothecin and cisplatin resistant cells (named as HCT116 IC60 CPT^{R}, HCT116 IC60 CDDP^{R} respectively), HCT116 wild type were seeded in a 10 cm dish to a confluency of 80%. The IC20 concentrations of cisplatin (3.38 μM) and camptothecin (12.6 nM) was added for 2 hours and 6 hours, respectively. Post treatment, the cells were washed twice with 1×PBS and fresh media was added. After every 48 hours, the media was changed and IC20 concentration of the drugs were added only if the confluency of cells was at least 80%. The IC20 resistant HCT116 cells was established after 4 rounds after checking the increase in the resistant index as determined using MTT. Similarly, IC40 HCT116 (15.5 μM of cisplatin and 30.8 nM of camptothecin) as well as IC60 HCT116 resistant cells (resistant to 35.09 μM cisplatin and 92.6 nM of camptothecin, respectively) are also generated following the same protocol. Resistant index was calculated using the following formula: Resistant index=IC60 of the resistant cells/IC60 of the parental cells. HCT116 IC60 1-OHP^{R} cells (Yang et al., 2006) were resistant to 2 μM of oxaliplatin.

Generation of sgRNA-Cas9 Expressing Vectors

The homozygous transversion from C>A at nucleotide 1784 (exon 7) in the BLM gene in GM03509 BS cell line was corrected using oligomer-based gRNA-CRISPR-Cas9 lentivirus approach. The approach is based on two sets of LR Gateway compatible plasmids; sgRNAs pDonor and a lentiviral expression plasmid created in this study (PHASE-DEST-CAS9-P2A-GFP). To clone the BLM specific sgRNA targeting the region of interest within BLM gene locus, one predicted sgRNA (CACTGGAAGACAGTCTGTCT) near to the site of interest was selected. A set of forward and reverse 25 bases long oligomers representing the selected sgRNA; CACCGCACTGGAAGACAGTCTGTCT and its inverse complementary primer; AAACAGACAGACTGTCTTCCAGTGC were custom synthesized. The forward and reverse oligonucleotides were mixed at equimolar concentrations, phosphorylated, annealed, ligated to BbsI digested pDonor and transformed in TOP10 cells and plated onto the Kanamycin selection plate. Positive clones of BLM specific gRNAs in pDonor vector were identified through Sanger sequencing and were further shuttled efficiently to the DEST containing lentiviral CAS9 expression plasmid (PHASE-DEST-CMV-CAS9-P2A-GFP) using LR-gateway reaction.

### Generation of Virus

Viral particles were produced by co-transfection of lentiviral expression plasmid (PHASE-gRNA-CMV-CAS9-P2A-GFP) together with packaging plasmids (pMDLg/pRRE, pRSV/REV, pMD2.G/V-SVG) [1 (8 μg):2 (4 μg):3 (2.66 μg) plasmid ratio) in Lenti-X HEK293T cell line using Xfect transfection reagent (1:0.5 ratio) in a 10 cm plates. Viral supernatants were collected 72 h post transfection and filtered using 0.45 μm filter followed by concentration using LentiX concentrator and titrated by qPCR (determination of number of transducing or infectious units per ml) on Hela cells. Titres for lentivirus were 6×10⁷ TU/ml. The cleavage efficiency of the sgRNA was determined by performing Surveyor assay using Surveyor Mutation Detection Kit as per manufacturer instructions.

### Generation of Double Cut Donor Vector

The double cut HDR donor vector was generated as described (Zhang et al., 2017). To obtain the HDR donor vector the following steps were sequentially performed (a) antisense strand of BLM sgRNA and the flanking PAM sequence was cloned into the XhoI/HindIII sites of mCherry2-C1; (b) sense strand of BLM sgRNA and the flanking PAM sequence was cloned into the KpnI/BamH1 sites of mCherry2-C1; (c) 1200 bp region spanning the site of mutation was PCR amplified from HCT116 WT genomic DNA was cloned into pUC18 at HindIII/KpnI sites, followed by mutating the PAM sequence for the sgRNA; (d) subcloning of the 1200 bp wildtype BLM sequence from pUC18 into mCherry2-C1 using HindIII/KpnI enzymes to generate the double cut donor vector.

### Establishment of CRISPR/Cas9 Mediated BLM Corrected BS Cell Line

GM03509 cells were transduced with the lentiviral vector expressing BLM sgRNA and GFP Cas9 at MOI of 0.1 using 10 μg/ml of polybrene. After 48 hours of transduction, 10 μg of double cut donor vector. The cells positive for mCherry and GFP were flow sorted and single cell was seeded in each well of the 96-well plate. The clones were screened for correction in the mutation by T7 enzyme mismatch cleavage (EMC) assay or surveyor analysis. PCR product of clone GM03509 BLM Clone 9.6 was cloned into TA vector and Sanger sequencing was carried out for 20 sub-clones which confirmed the correction in the genome of GM03509 cells. The expression of BLM in GM03509 BLM Clone 9.6 was then examined using the western blot and immunoflorescence assays. The functionality of the expressed BLM in GM03509 BLM Clone 9.6 cells was demonstrated by SCE assays.

### Small Molecule Library, Peptides

Each of the small molecules in Prestwick Chemical Library were dissolved in DMSO to make 100 mM stocks. For in vitro assays subsequent dilutions were made in 1×PBS, while for in vivo studies, the compounds were dissolved in 1×PBS containing 5% DMSO. The sequence encoding BLM (181-212) was: STAQKSKKGKRNFFKAQLYTTNTVKTDLPPPS (designated as BLM_peptide). A control peptide sequence, ALGFTDQKTPKSKRTVNSKQPFKTPSKNLTAY (designated as the SCM_peptide) was designed which had the same amino acid composition and did not bear homology to any known protein in human protein database. The Cell Permeable Peptide (CPP) versions (BLM_CPP and SCM_CPP) were tagged with SV40 Large T antigen Nuclear localization Signal (PKKKRKVEDPYC) in the N-terminus and TAMRA in the C-terminus. The biotinylated version of BLM_peptide was named as Biotin BLM_Peptide.

### Renilla Luciferase Based PCA

Renilla Luciferase (Rlu) based Protein complementation assay was carried out as described in (Stefan et al., 2007). For preliminary screening of the Prestwick Chemical Library (containing 1280 FDA/EMA approved small molecules), HEK293T cells were seeded in the 6-well plates a day prior to the transfection. Next day the cells were transfected by 2 μg each of pcDNA3.1 N-RAD54 F1, pcDNA3.1 C-RAD54 F1, pcDNA3.1 BLM F1, pcDNA3.1 BLM F2 plasmids (alone or in different combinations) using Lipofectamine 2000 (according to the manufacturer's protocol). 36 hours post-transfection, approximately 105 cells were seeded in a 96-well plate and incubated at 37° C. for 2 hour with or without 10 μM of the 1280 small molecules present in the library. 10 μM of Coenleterazine h was added into each well and Rluc activity was monitored for the first 10 seconds in Varioskan Microplate Reader (Thermo Fisher Scientific). The validation screen was performed with 17 small molecules at 100 μM, 10 μM, 1 μM, 100 nM, 10 nM and 1 nM concentrations. The percentage reduction in the protein-protein interaction was then calculated as compared to the control wells (without any small molecule).

### Purification and In Vitro Interactions

All GST-tagged and His tagged proteins were expressed in E. coli BL-21 DE3 competent cells at 16° C. overnight and subsequently purified according to standard protocols by binding to either Glutathione-S-Sepharose (for GST-tagged proteins) or Nickel-NTA beads (for His tagged proteins). The bound proteins were eluted out using either 2 mM reduced glutathione (for GST-tagged proteins) or 200 mM imidazole (for His tagged proteins). The eluted fractions were pooled, dialysed and used for the assays.

### Chromatin Remodeling and Restriction Enzyme Accessibility Assay

The p2085S-G5E4 was digested with Asp718 and ClaI to release the 2.5 kb insert. pUC57-MRP2 array was digested with EcoR1 and HindIII to release the 2.6 kb insert. The released inserts fragments were end labelled with [□³²P]ATP using T4 Polynucleotide Kinase enzyme. Nucleosomal reconstitution on the purified fragments were carried out using gradient salt dialysis method in presence of HeLa core histones. The radiolabelled reconstituted arrays were checked (in a 1% agarose gel), and subsequently used in restriction enzyme accessibility (REA) assays. For each reaction, 500 nM RAD54 (wildtype or mutants), 500 nM BLM protein (wildtype or mutants) or 180 nM BLM_peptide or SCM_peptide, radiolabelled array (1 nM) were used in 1×REA buffer (20 mM HEPES, pH 7.9; 40 mM KCl, 4 mM MgCl₂) in the presence of 0.4 U/μl of HhaI. RAD51-ssDNA (500 nM-1.5 □M) was added where required. The concentration of ATP used in the reactions was 2 mM. C3, C7, C17 were added in the reaction mixture at a concentration of 10 μM. The reactions were incubated at 30° C. for different time intervals (as indicated in the figure legends). RAD51-ssDNA was prepared using 500 nM of RAD51 and 1.5 □M of oligonucleotide (molar concentration of nucleotides) as described in (Zhang et al., 2007). The sequence of the oligonucleotide used was oligonucleotide 2 in (Zhang et al., 2007). BLM-RAD54 disruptors were added at final concentration of 10 μM). The reactions were stopped by addition of the stop buffer (50 mM EDTA, 1.2% SDS), digested with Proteinase K (1 mg/ml) at 37° C. for 30 minutes. Proteins were removed by phenol-chloroform extraction, DNA was ethanol precipitated, washed and analysed on 1% agarose gels. The intensity of uncut products was measured from Phosphoimager scans taken in Typhoon™ laser-scanner platform (Cytiva) and ratio of the cut and the uncut products was quantitated using the inbuilt software.

### Characterization

### ATPase Assay

Assay for RAD54 ATPase activity was carried out as per the previously published protocol (Srivastava et al., 2009). His-RAD54 (120 nM) was incubated with the GST-BLM (1-212) or BLM_peptide or SCM_peptide (180 nM). Where gradient of proteins were used, the concentrations have been indicated in the figure legends. C3, C7, C17 were added in the reaction mixture at a concentration of 10 μM. φX174RF1 DNA (22 μM base pairs) was added to initiate the ATP hydrolysis reaction at 30° C. for 15 minutes and 20 nCi of [γ³²P]ATP. 1 μl of the sample was spotted on the polyethyleneimine-coated TLC plate, resolved in 1M Formic Acid and 0.3M LiCl. The products of ATP hydrolysis were then visualized by Phosphoimager scans taken in Typhoon™ laser-scanner platform (Cytiva) and quantitated using the inbuilt software.

### ATP Binding Assay

A 20 μl reaction was set up with 120 nM of GST-RAD54 and 180 nM of either BLM_peptide or SCM_peptide or BLM (1-212) using 4 μl of 5×ATP binding assay buffer (100 mM Tris-Cl pH 7.5, 50 mM MgCl₂, 10 mM MnCl₂, 5 mM DTT) and 5 μCi of [γ³²P]dATP. C3, C7, C17 were added in the reaction mixture at a concentration of 10 μM. Reactions were incubated at 30° C. for 20 minutes and were stopped with 10% TCA and samples were spotted on P81 phosphocellulose paper followed by washing of paper strips with 75 mM orthophosphoric acid. A minimum of 8 washes were given in 20 hrs of 1 hr each including a 12 hr wash. Following this, strips were dehydrated in ethanol for 10 min followed by liquid scintillation counting. Reactions without the substrate were spotted as control and the same process repeated.

### In Vitro Interaction Assays

pcDNA3.1 Flag RAD54 was used for coupled in vitro transcription/translation of RAD54 using T7 Quick coupled Transcription/Translation System kit S³⁵ methionine. Interaction between bound GST-BLM (wildtype or mutants) and radiolabelled RAD54 were carried out as described previously (Gupta et al., 2014). Interactions between with bound GST-RAD54 or His-RAD54 and GST-BLM were carried out at 4° C. in 500 μl of GST buffer (50 mM Tris pH 7.5, 100 mM KCl, 10 mM MgCl₂, 5% glycerol and 0.5% NP-40) for 2 hours in absence or presence of C3, C7, C17, after which the beads were washed twice with GST buffer. 10 μM of the 3 compounds were used in the in vitro interaction assays.

### Bio-Layer Interferometry (BLI)

The interactions between RAD54 and biotinylated BLM_peptide (181-212) as well as the various compounds were studied using Bio-layer interferometry (BLI) using the Octet K2 system (ForteBio Systems). Approximately, 250 μg of His-tagged RAD54 was immobilized onto a Ni-NTA chip for 600 s and the unbound protein was washed using 10 mM HEPES pH 7.5 buffer. Different concentrations of the biotinylated BLM (181-212) peptide (dissolved in water) and C3, C7, and C17 were prepared and loaded in the 96-well plate. Each of the reaction set was then used to check for their association with RAD54 captured on the sensor. The kinetics of the RAD54-BLM interaction was monitored for 180 s each for the association and the dissociation curves by dipping the sensors in a series of increasing concentrations of the BLM_peptide. 500 mM NaCl was used as the dissociation buffer. A control sensor with the immobilized RAD54 was used in parallel and was used to normalize the data. Washing in 350 mM EDTA regenerated the sensors for the next cycle of binding kinetics. The experiments were performed at 25° C. All the real-time data was analysed and KD values were determined using the Octet® Evaluation software using 1:1 fitting curves.

RT-qPCR, ChIP-qPCR, Re-ChIP qPCR

Total RNA was isolated from cells and tissues using TRIzol reagent containing 1% □-mercaptoethanol. cDNA was generated using Reverse transcriptase core kit according to manufacturer's protocol. RAD54 and BLM ChIP have been carried out according to published procedures (Priyadarshini et al., 2018; Schmidt et al., 2009). The sequential ChIP were performed according to a published protocol (Furlan-Magaril et al., 2009). The concentrations of input samples, ChIP DNA, Re-ChIP DNA were determined by Qubit using dsDNA HS assay kit. 1 ng of ChIP DNA or Re-ChIP DNA was used for qPCR with primers which amplify MRP2, MRP3 and MDR1 promoters. GAPDH primer was used as the internal control. All qPCR reactions were carried out in were carried out in QuantStudio 3 Real-time PCR system.

### ChIP Sequencing

The ChIP DNA libraries were constructed using NEBNext Ultra II DNA Library Prep with Sample Purification Beads (Catalog number-E7103L) according to the manufacturer's instructions. Next generation sequencing of libraries was performed using Illumina HiSeq 2500 rapid run V2 kit for 1×50 bp. The quality of the raw reads were determined using FastQC (http://www.bioinformatics.babraham.ac.uk/projects/fastqc/), followed by removal adaptors using Trimmomatic (Bolger et al., 2014). Bases with high Phred score (more than 30) were aligned to the current human genome (hg38 assembly) using Bowtie 1 (Langmead et al., 2009). Unique reads were then analyzed to determine the BLM enrichment on TSS as described (Bardet et al., 2011; Priyadarshini et al., 2018). The data was visualized as circos using RStudio and IGV tools.

### Electrophoretic Mobility Shift Assay (EMSA)

Assays were carried out according to a previously published protocol (Gupta et al., 2014), with minor modifications. For the reactions, the indicated recombinant proteins and a radiolabelled G5E4 array (1 nM) were used. The proteins and the radiolabelled array were incubated in a buffer [20 mM HEPES (pH 7.9), 40 mM KCl, 2 mM ATP and 4 mM MgCl₂] at 30° C. for 30 min. Reactions were stopped by adding ADP and salmon sperm DNA to final concentrations of 5 mM and 100 μg/ml, respectively. The products were resolved on 0.6% agarose gel. The gels were dried and products analyzed in phosphoimager.

### Tryptophan Fluorescence Assay

Tryptophan fluorescence measurements were performed on a Fluoromax Spectrofluorimeter (Horiba-Jobin Yvon Fluoromax 4). Recombinant His-RAD54 (500 nM) was excited at 280 nm and the emission intensity monitored at regular intervals from 295 nm to at least 450 nm. The emission spectra of RAD54 was determined either (a) alone or (b) in presence of BLM_peptide or SCM_peptide (c) in presence of C3, C7, C17. The concentrations used for RAD54, BLM_peptide, SCM_peptide, C3, C7, C17 are presented in the figure legends.

### Chromatin Fractionation

Chromatin fractionation was performed according to the protocol described in (Lou et al., 2006) with few modifications. Briefly, one 10 cm dish of HCT116 IC60 CPT^{R} cells were lysed in Buffer I (50 mM HEPES, pH 7.5, 150 mM NaCl, 1 mM EDTA, 0.05% NP40 and protease and phosphatase inhibitors) for 2 minutes on ice. Cell lysates were centrifuged at 1575 g for 5 minutes at 4° C. The soluble proteins were collected as Fraction I. The precipitate obtained after centrifugation was washed once with Buffer I (designated as Fraction II), then extracted with Buffer II (50 mM Tris-HCl, pH 7.5, 150 mM NaCl, 1% NP40, 0.5% sodium deoxycholate, 0.1% SDS and protease and phosphatase inhibitors) on ice for 30 minutes with intermittent vortexing. The extracts were then centrifuged at 25,200 g for 20 minutes at 4° C. and supernatants were collected as the chromatin bound protein fraction (Fraction III).

### MTT Assay

Cells were seeded in a 96-well plate overnight. Next day, the culturing media was replaced with Optimem media, followed by incubation with BLM_CPP or SCM_CPP for 1 hr. The final concentrations of the peptides were 450 nM for cisplatin (CDDP) and 180 nM (for all other drugs). The cells were then incubated with 100 nM of the C3, C7 and C17 along with gradient of the drugs for 2 hours (for CDDP) or 6 hours (for all other drugs). The IC60 concentrations of C3, C7 and C17 for each cell type was used. Post treatment, the cells were washed with 1×PBS and fresh media was added. After 72 hours, MTT assay was carried out to determine the percentage cell viability of the cells with respect to the untreated cells. Cells without any drug but in presence of C3, C7, C17 were also kept to examine the effect of these compounds alone on the cell viability.

### Alkaline Comet Assay

Alkaline Comet assay was carried out according to the published protocol (Olive and Banath, 2006). For the assay, GM03509 GFP-BLM and GM03509 GFP cells were treated with HU treatment (16 hours). Cells released post HU treatment were grown for 6 hours with 180 nM of BLM_CPP or SCM_CPP, following which Comet assays were carried out. For each experiment 2000 cells were used.

### Soft Agar Assay

3000 cells mixed with 0.4% agar and seeded on top of the 6-well plates pre-coated with 0.8% agar. The resistant cells were treated with 120 nM of CPT. Post-incubation cells were seeded. The media containing 100 nM of C3, C7, C17 was added at the start and replenished after every 3 days. The colonies were then counted after 15 days after staining with 0.25% Crystal Violet.

### HR Assay and SCE

For HR assay, 10 μg of the HR plasmid (Seluanov et al., 2010) was digested with I-SceI, purified and eluted into 20 μl of 10 mM TrisHCl pH 8.0. 5 μg of the linearized reporter cassette along with 100 ng of tdTomato-N1 plasmid were transfected in an exponentially growing 10 cm dish of cells using FuGENE® HD Transfection Reagent. After 3 days of transfection, the cells were harvested. The GFP positive and TdRed positive cells were analysed by FACS. SCE assays were carried out as described earlier (Chabosseau et al., 2011).

### Preparation of Lipid Nanoparticles of Camptothecin (CPT_NPs) and Peptides (BLM_NPs, SCM_NPs)

Egg PC (1 mg), cholesterol (0.3 mg), DSPE-PEG2000-Amine (0.2 mg), Camptothecin (0.5 mg) were mixed in chloroform (0.2 mL) in 1:0.3:0.2:0.5 weight ratio in a round bottom Wheaton glass vial. Chloroform was evaporated by using stream of nitrogen to form a dry thin film. In case of BLM_NPs and SCM_NPs, EggPC (1 mg), cholesterol (0.5 mg), and DSPE- PEG2000-Amine (0.2 mg) were used to form the thin film by above mentioned method, and thin films were kept under vacuum for overnight. Thin films were then hydrated at 4° C. for 6 h with 1 mL saline (for Camptothecin liposomes) or 1 mL saline containing either BLM_peptide or scrambled (SCM) peptide. These lipid suspensions were sonicated in a bath sonicator for 2 min and extruded through 200 nm (14 times each) polycarbonate membranes. Free Camptothecin or peptide was removed by passing through 1 mL Sephadex G-25 bed. Encapsulation efficiency (concentration of Camptothecin or peptide in liposomes) was determined using absorbance method. Liposomes (10 μL) were dissolved in methanol (190 μL), followed by measurement of Camptothecin absorbance at 370 nm or peptide absorbance at 275 nm.

### Hydrogel Preparations of Camptothecin and BLM_Peptide

For animal experiments, we used hydrogel based localized delivery of camptothecin and BLM or SCM peptide. We prepared the camptothecin entrapped gel (CPT-Gel), camptothecin and BLM_peptide entrapped gel (CPT-BLM-Gel), and camptothecin and scrambled (SCM) peptide gel (CPT-SCM-Gel) using a lithocholic acid-derived hydrogelator (Pal et al., 2019). Typically, camptothecin (5 mg) and 130 mg of gelator in 2 mL autoclaved water was heated to form clear solution. For combination hydrogels, 2.5 mg of peptide was added to heated solution containing Camptothecin. Solution was then sonicated and allowed to cool at room temperature to form hydrogel, and 0.2 mL of hydrogel was injected in each mouse near the tumor site.

### Animal Studies

All animal studies were carried out in National Institute of Immunology according to approved animal ethics protocols (IAEC #357/14, IAEC #398/15, IAEC #567/20). To determine whether CPT-BLM-Gel enhanced tumor growth in a xenograft mice model, 3×106 HCT116 BLM−/− cells mixed with Matrigel cells were injected into either Nude mice or SCID mice. The day when the approximate volume of the tumor was 50 mm3 was considered as Day 1. On Day 1, treatment with CPT-Gel was initiated at the base of the tumors either alone or along with the injection of either CPT-BLM-Gel or CPT-SCM-Gel.

To authenticate the effect of BLM (181-212) on tumor formation, xenograft studies were carried out in SCID mice with two stable lines in which either GFP or GFP NLS BLM (181-212) were expressed in HCT116 BLM−/− cells. 3×106 cells were mixed with Matrigel (1:1 ratio) and then injected subcutaneously.

To determine the effect of C3, C7, C17 on their ability to diminish tumor formation, the xenograft models were carried out in SCID and NSG mice using HCT116 WT IC60CPTR or HCT116 1-OHPR cells, respectively. Upon tumor formation (˜50 mm3), either CPT (1.25 mg/kg) alone, C3/C7/C17 alone (5 mg/kg) or CPT and C3/C7/C17 in combination were administered intraperitoneally after every 3 days. For experiments with HCT116 1-OHPR cells, 1-OHP was administered at 2 mg/kg dose either alone in combination with C3/C7/C17 (5 mg/kg). In all cases tumor volume were measured at the indicated days post-injection using the following formula: Tumor volume=½(length×width2).

### Immunofluorescence and TUNEL Assay

To determine the effect of BLM_CPP or SCM_CPP on RAD51, RAD54 and γH2AX foci number, HCT116 BLM−/− cells were treated with HU for 16 hours. Cells were washed with 1×PBS and growth continued for an additional 6 hours with either 180 nM BLM_CPP or SCM_CPP. Cells were fixed with 4% paraformaldehyde and processed for immunofluorescence using antibodies against RAD51, RAD54 or γH2AX. Imaging and subsequent analysis was done in a motorized epifluorescence microscope (Upright Axioimager M1; Carl Zeiss) as previously described (Tripathi et al., 2007).

For immunofluorescence in tumors, the samples were fixed in 10% neutral buffered formalin and paraffin embedded blocks was prepared. For immunofluorescence, 2-micron tissues sections were first deparaffinized using xylene for 20 minutes, followed by rehydration by immersing in 100%, 90% and 70% ethanol for 10 minutes each, followed by washing with water. The antigen retrieval was then done using sodium citrate buffer (10 mM Sodium Citrate, pH 6.0) in a decloaking chamber (Biocare Medical) for 10 minutes at 95° C. followed by 70° C. for 5 minutes. Tissue sections were permeabilized with 1% goat serum and 0.4% Triton X-100 in PBS, blocked with 5% goat serum in 0.01% PBS supplemented with Tween-20 (PBS-T) for 1 hour at room temperature and stained with Ki67 antibody overnight at 4° C. Next day, sections were washed twice with 1% goat serum in PBS-T for 10 minutes each and stained with Alexa 488 labelled secondary antibody for 1 hour. Post two washes, the nuclei were stained with DAPI.

### TUNEL Assay

TUNEL assay was carried using Fluorometric TUNEL System according to the manufacturer's instructions. Briefly, the deparaffinised, rehydrated tissue sections were fixed 4% formaldehyde in PBS for 15 minutes and then permeabilized using 100 μl of a 20 μg/ml Proteinase K solution incubated at room temperature for 8-10 minutes. Following this, the sections were refixed for 5 minutes, equilibrated and then 50 μl of TdT reaction mix was added for 60 minutes at 37° C. in a humidified chamber. The reaction was stopped by immersing slides in 2×SSC for 15 minutes. The nuclei were the counterstained with DAPI. All imaging was carried out in LSM 510 Meta System (Carl Zeiss, Germany) with 63×/1.4 oil immersion objective. The laser line used was Argon 458/477/488/514 nm.

### Statistical Analysis

All quantitations are presented as mean±S.D. Details about the number of samples analyzed for each experiment are mentioned in figure legends. The p values or calculated probability is as follows: * p<0.05, ** p<0.01, *** p<0.001, **** p<0.0001 while n.s. indicates that the result is not significant.

### Numbered Embodiments

1. A method for treating colorectal cancer in a patient in need thereof, comprising:
a. administering a chemotherapy to the patient; and
b. administering an inhibitor of Bloom syndrome protein (BLM) and RAD54 interaction to the patient after starting or simultaneously with the chemotherapy.
2. The method as recited in Embodiment 1, wherein the inhibitor of BLM-RAD54 interaction is selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.
3. The method as recited in Embodiment 1 or 2, wherein the inhibitor of BLM-RAD54 interaction is selected from Acetazolamide or a pharmaceutically acceptable salt thereof, Dipyridamole or a pharmaceutically acceptable salt thereof, Loxapine Succinate, or a combination thereof.
4. The method as recited in any one of Embodiments 1-3, wherein the chemotherapy comprises administration of cisplatin, oxaloplatin, carboplatin, camptothecin, or a combination thereof.
5. The method as recited in any one of Embodiments 1-4, wherein the chemotherapy comprises administration of 5-Fluorouracil (5-FU), Capecitabine, Irinotecan, Oxaliplatin, Trifluridine, tipiracil, or a combination thereof.
6. The method as recited in any one of Embodiments 1-5, wherein administration of the inhibitor of BLM-RAD54 interaction inhibits the interaction of BLM with RAD54 in cancer cells of the patient by about 10%-80% compared to levels of BLM-RAD54 interaction in the absence of administration of the inhibitor.
7. The method as recited in any one of Embodiments 1-6, wherein administration of the inhibitor of BLM-RAD54 interaction reduces proliferation of colorectal cancer cells by about 10%-80% in the patient compared to proliferation of colorectal cancer cells in the absence of administration the inhibitor.
8. A method for inhibiting an interaction of Bloom syndrome protein (BLM) and RAD54 in cancer cells, comprising contacting the cancer cells with an inhibitor of BLM-RAD54 interaction selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.
9. The method as recited in Embodiment 8, wherein the inhibitor of BLM-RAD54 interaction is selected from Acetazolamide or a pharmaceutically acceptable salt thereof, Dipyridamole or a pharmaceutically acceptable salt thereof, Loxapine Succinate, or a combination thereof.
10. The method as recited in Embodiment 8 or 9, wherein the cancer cells are being administered with cisplatin, oxaloplatin, carboplatin, camptothecin, or a combination thereof.
11. The method as recited in any one of Embodiments 8-10, wherein the cancer cells are colorectal cancer cells.
12. The method as recited in any one of Embodiments 8-11, wherein the interaction of BLM with RAD54 is inhibited by about 10%-80% in the cancer cells compared to untreated cancer cells or cancer cells treated with a control.
13. The method as recited in any one of Embodiments 8-12, wherein the inhibitor of BLM-RAD54 interaction reduces proliferation of cancer cells by about 10%-80% compared to untreated cancer cells or cancer cells treated with a control.
14. A method for reducing resistance to chemotherapy in a cancer patient, comprising administering an inhibitor of BLM-RAD54 interaction to the cancer patient after starting the chemotherapy or simultaneously with the chemotherapy.
15. The method as recited in Embodiment 14, wherein the inhibitor of BLM-RAD54 interaction is selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.
16. The method as recited in Embodiment 14 or 15, wherein the inhibitor of BLM-RAD54 interaction is selected from Acetazolamide or a pharmaceutically acceptable salt thereof, Dipyridamole or a pharmaceutically acceptable salt thereof, Loxapine Succinate, or a combination thereof.
17. The method as recited in any one of Embodiments 14-16, wherein the chemotherapy comprises administration of cisplatin, oxaloplatin, carboplatin, camptothecin, or a combination thereof.
18. The method as recited in any one of Embodiments 14-17, wherein the cancer patient has colorectal cancer.
19. The method as recited in any one of Embodiments 14-18, wherein the BLM-RAD54 interaction is inhibited by about 10%-80% in the cancer patient compared to levels of BLM-RAD54 interaction in the absence of the inhibitor.
20. The method as recited in any one of Embodiments 14-19, wherein administration of the inhibitor of BLM-RAD54 interaction reduces proliferation of cancer cells by about 10%-80% in the patient compared to proliferation of cancer cells in the absence of the inhibitor.
21. An inhibitor of Bloom syndrome protein (BLM) and RAD54 interaction for use as an adjunct therapy in treating cancer.
22. The inhibitor for use as recited in Embodiment 21, wherein the cancer is colorectal cancer.
23. The inhibitor for use as recited in Embodiment 21 or 22, wherein the inhibitor is selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.
24. The inhibitor for use as recited in any one of Embodiments 21-23, wherein the inhibitor is selected from Acetazolamide or a pharmaceutically acceptable salt thereof, Dipyridamole or a pharmaceutically acceptable salt thereof, Loxapine Succinate, or a combination thereof.
25. The inhibitor for use as recited in any one of Embodiments 21-24, wherein the inhibitor is an adjunct therapy for chemotherapy comprising cisplatin, oxaloplatin, carboplatin, camptothecin, or a combination thereof.
26. The inhibitor for use as recited in any one of Embodiments 21-24, wherein the inhibitor is an adjunct therapy for chemotherapy comprising administration of 5-Fluorouracil (5-FU), Capecitabine, Irinotecan, Oxaliplatin, Trifluridine, tipiracil, or a combination thereof.
27. The inhibitor for use as recited in any one of Embodiments 21-26, wherein the inhibitor inhibits the interaction of BLM and RAD54 in cancer cells by about 10%-80% compared to levels of BLM-RAD54 interaction in the absence of the inhibitor.
28. The inhibitor for use as recited in any one of Embodiments 21-27, wherein the inhibitor reduces proliferation of cancer cells by about 10%-80% compared to proliferation of cancer cells in the absence the inhibitor.
29. An inhibitor of Bloom syndrome protein (BLM) and RAD54 interaction for use in reducing resistance to chemotherapy in treating cancer.
30. The inhibitor for use as recited in Embodiment 29, wherein the cancer is colorectal cancer.
31. The inhibitor for use as recited in Embodiment 29 or 30, wherein the inhibitor is selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.
32. The inhibitor for use as recited in any one of Embodiments 29-31, wherein the inhibitor is selected from Acetazolamide or a pharmaceutically acceptable salt thereof, Dipyridamole or a pharmaceutically acceptable salt thereof, Loxapine Succinate, or a combination thereof.
33. The inhibitor for use as recited in any one of Embodiments 29-32, wherein the chemotherapy comprises cisplatin, oxaloplatin, carboplatin, camptothecin, or a combination thereof.
34. The inhibitor for use as recited in any one of Embodiments 29-32, wherein the chemotherapy comprises 5-Fluorouracil (5-FU), Capecitabine, Irinotecan, Oxaliplatin, Trifluridine, tipiracil, or a combination thereof.
35. The inhibitor for use as recited in any one of Embodiments 29-34, wherein the inhibitor inhibits the interaction of BLM and RAD54 in cancer cells by about 10%-80% compared to levels of BLM-RAD54 interaction in the absence of the inhibitor.
36. The inhibitor for use as recited in any one of Embodiments 29-35, wherein the inhibitor reduces proliferation of cancer cells by about 10%-80% compared to proliferation of cancer cells in the absence of the inhibitor.
37. Use of an inhibitor of Bloom syndrome protein (BLM) and RAD54 interaction as an adjunct therapy in treating cancer.
38. The use as recited in Embodiment 37, wherein the cancer is colorectal cancer.
39. The use as recited in Embodiment 37 or 38, wherein the inhibitor is selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.
40. The use as recited in any one of Embodiments 37-39, wherein the inhibitor is selected from Acetazolamide or a pharmaceutically acceptable salt thereof, Dipyridamole or a pharmaceutically acceptable salt thereof, Loxapine Succinate, or a combination thereof.
41. The use as recited in any one of Embodiments 37-40, wherein the inhibitor is an adjunct therapy for chemotherapy comprising cisplatin, oxaloplatin, carboplatin, camptothecin, or a combination thereof.
42. The use as recited in any one of Embodiments 37-40, wherein the inhibitor is an adjunct therapy for chemotherapy comprising 5-Fluorouracil (5-FU), Capecitabine, Irinotecan, Oxaliplatin, Trifluridine, tipiracil, or a combination thereof.
43. The use as recited in any one of Embodiments 37-42, wherein the inhibitor inhibits the interaction of BLM and RAD54 in cancer cells by about 10%-80% compared to levels of BLM-RAD54 interaction in the absence of the inhibitor.
44. The use as recited in any one of Embodiments 37-43, wherein the inhibitor reduces proliferation of cancer cells by about 10%-80% compared to proliferation of cancer cells in the absence of the inhibitor.
45. Use of an inhibitor of Bloom syndrome protein (BLM) and RAD54 interaction in reducing resistance to chemotherapy in treating cancer.
46. The use as recited in Embodiment 45, wherein the cancer is colorectal cancer.
47. The use as recited in Embodiment 45 or 46, wherein the inhibitor is selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.
48. The use as recited in any one of Embodiments 45-47, wherein the inhibitor is selected from Acetazolamide or a pharmaceutically acceptable salt thereof, Dipyridamole or a pharmaceutically acceptable salt thereof, Loxapine Succinate, or a combination thereof.
49. The use as recited in any one of Embodiments 45-48, wherein the chemotherapy comprises cisplatin, oxaloplatin, carboplatin, camptothecin, or a combination thereof.
50. The use as recited in any one of Embodiments 45-48, wherein the chemotherapy comprises 5-Fluorouracil (5-FU), Capecitabine, Irinotecan, Oxaliplatin, Trifluridine, tipiracil, or a combination thereof.
51. The use as recited in any one of Embodiments 45-50, wherein the inhibitor inhibits the interaction of BLM and RAD54 in cancer cells by about 10%-80% compared to levels of BLM-RAD54 interaction in the absence of the inhibitor.
52. The use as recited in any one of Embodiments 45-51, wherein the inhibitor reduces proliferation of cancer cells by about 10%-80% compared to proliferation of cancer cells in the absence the inhibitor.

1. A method for treating colorectal cancer in a patient in need thereof, comprising:
a. administering a chemotherapy to the patient; and
b. administering an inhibitor of Bloom syndrome protein (BLM) and RAD54 interaction to the patient after starting the chemotherapy or simultaneously with the chemotherapy.

2. The method of claim 1, wherein the inhibitor of BLM-RAD54 interaction is selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.

3. The method of claim 1, wherein the inhibitor of BLM-RAD54 interaction is selected from Acetazolamide or a pharmaceutically acceptable salt thereof, Dipyridamole or a pharmaceutically acceptable salt thereof, Loxapine Succinate, or a combination thereof.

4. The method of claim 1, wherein the chemotherapy comprises administration of cisplatin, oxaloplatin, carboplatin, camptothecin, or a combination thereof.

5. The method of claim 1, wherein the chemotherapy comprises administration of 5-Fluorouracil (5-FU), Capecitabine, Irinotecan, Oxaliplatin, Trifluridine, tipiracil, or a combination thereof.

6. The method of claim 1, wherein administration of the inhibitor of BLM-RAD54 interaction inhibits the interaction of BLM and RAD54 in cancer cells of the patient by about 10%-80% compared to levels of BLM-RAD54 interaction in the absence of administration of the inhibitor.

7. The method of claim 1, wherein administration of the inhibitor of BLM-RAD54 interaction reduces proliferation of colorectal cancer cells by about 10%-80% in the patient compared to proliferation of colorectal cancer cells in the absence of administration the inhibitor.

8. A method for inhibiting an interaction of Bloom syndrome protein (BLM) and RAD54 in cancer cells, comprising contacting the cancer cells with an inhibitor of BLM-RAD54 interaction selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.

9. The method of claim 8, wherein the inhibitor of BLM-RAD54 interaction is selected from Acetazolamide or a pharmaceutically acceptable salt thereof, Dipyridamole or a pharmaceutically acceptable salt thereof, Loxapine Succinate, or a combination thereof.

10. The method of claim 8, wherein the cancer cells are being administered with cisplatin, oxaloplatin, carboplatin, camptothecin, or a combination thereof.

11. The method of claim 8, wherein the cancer cells are colorectal cancer cells.

12. The method of claim 8, wherein the interaction of BLM and RAD54 is inhibited by about 10%-80% in the cancer cells compared to untreated cancer cells or cancer cells treated with a control.

13. The method of claim 8, wherein the inhibitor of BLM-RAD54 interaction reduces proliferation of cancer cells by about 10%-80% compared to untreated cancer cells or cancer cells treated with a control.

14. A method for reducing resistance to chemotherapy in a cancer patient, comprising administering an inhibitor of BLM-RAD54 interaction to the cancer patient after starting the chemotherapy or simultaneously with the chemotherapy.

15. The method of claim 14, wherein the inhibitor of BLM-RAD54 interaction is selected from the group consisting of: Azaguanine-8, Allantoin, Acetazolamide, Metformin, Atracurum, Prednisone, Dipyridamole, Metronidazole, Khellin, Apomorphine, Naloxone, Bromocryptine, Glipizide, Verapamil, Erythromycin, Chloroxine, Loxapine, a pharmaceutically acceptable salt thereof, and a combination thereof.

16. The method of claim 14, wherein the inhibitor of BLM-RAD54 interaction is selected from Acetazolamide or a pharmaceutically acceptable salt thereof, Dipyridamole or a pharmaceutically acceptable salt thereof, Loxapine Succinate, or a combination thereof.

17. The method of claim 14, wherein the chemotherapy comprises administration of cisplatin, oxaloplatin, carboplatin, camptothecin, or a combination thereof.

18. The method of claim 14, wherein the cancer patient has colorectal cancer.

19. The method of claim 14, wherein the BLM-RAD54 interaction is inhibited by about 10%-80% in the cancer patient compared to levels of BLM-RAD54 interaction in the absence of the inhibitor.

20. The method of claim 14, wherein administration of the inhibitor of BLM-RAD54 interaction reduces proliferation of cancer cells by about 10%-80% in the patient compared to proliferation of cancer cells in the absence of the inhibitor.
